SEQUENCE LISTING (37 sequences)

SEQ ID NO: 1 - protein, 145 aa, Homo sapiens
MELALLCGLVVMAGVIPIQGGILNLNKMVKQVTGKMPILSYWPYGCHCGLGGRGQPKDATDWCCQTHDCCYDHLKTQGCSIYKDYYRYNFSQGNIHCSDKGSWCEQQLCACDKEVAFCLKRNLDTYQKRLRFYWRPHCRGQTPGC

SEQ ID NO: 2 - protein, 125 aa, Homo sapiens
GILNLNKMVKQVTGKMPILSYWPYGCHCGLGGRGQPKDATDWCCQTHDCCYDHLKTQGCSIYKDYYRYNFSQGNIHCSDKGSWCEQQLCACDKEVAFCLKRNLDTYQKRLRFYWRPHCRGQTPGC

SEQ ID NO: 3 - protein, 144 aa, Artificial Sequence
  Synthetic Construct
MELALLCGLVVMAGVIPIQGGILNLNKMVKQVTGKMPILSYWPYGCHCGLGGRGQPKDATDWCCQTADCCYDHLKTQGCSIYKDYYRYNFSQGNIHCSDKGSWCEQQLCACDKEVAFCLKRNLDTYQKRLRFYWRPHCRGQTGC

SEQ ID NO: 4 - protein, 124 aa, Artificial Sequence
  Synthetic Construct
GILNLNKMVKQVTGKMPILSYWPYGCHCGLGGRGQPKDATDWCCQTADCCYDHLKTQGCSIYKDYYRYNFSQGNIHCSDKGSWCEQQLCACDKEVAFCLKRNLDTYQKRLRFYWRPHCRGQTGC

SEQ ID NO: 5 - protein, 116 aa, Homo sapiens [sequence omitted]

SEQ ID NO: 6 - protein, 96 aa, Artificial Sequence
  Synthetic Construct
GILNLNKMVKQVTGKMPILSYWPYGCHCGLGGRGQPKDATDWCCQTHDCCYDHLKTQGCGIYKDNNKSSIHCMDLSQRYCLMAVFNVIYLENEDSE

SEQ ID NO: 7 - protein, 116 aa, Artificial Sequence
  Synthetic Construct
MELALLCGLVVMAGVIPIQGGILNLNKMVKQVTGKMPILSYWPYGCHCGLGGRGQPKDATDWCCQTADCCYDHLKTQGCGIYKDNNKSSIHCMDLSQRYCLMAVFNVIYLENEDSE

SEQ ID NO: 8 - protein, 96 aa, Artificial Sequence
  Synthetic Construct
GILNLNKMVKQVTGKMPILSYWPYGCHCGLGGRGQPKDATDWCCQTADCCYDHLKTQGCGIYKDNNKSSIHCMDLSQRYCLMAVFNVIYLENEDSE

SEQ ID NO: 9 - protein, 116 aa, Artificial Sequence
  Synthetic Construct
MELALLCGLVVMAGVIPIQGGILNLNKMVKQVTGKMPILSYWPYGCHCGLGGRGQPKDATDWCCQTHDCCYDHLKTQGCSIYKDNNKSSIHCMDLSQRYCLMAVFNVIYLENEDSE

SEQ ID NO: 10 - protein, 96 aa, Artificial Sequence; identical to SEQ ID NO: 6
  Synthetic Construct

SEQ ID NO: 11 - protein, 116 aa, Artificial Sequence
  Synthetic Construct
MELALLCGLVVMAGVIPIQGGILNLNKMVKQVTGKMPILSYWPYGCHCGLGGRGQPKDATDWCCQTADCCYDHLKTQGCSIYKDNNKSSIHCMDLSQRYCLMAVFNVIYLENEDSE

SEQ ID NO: 12 - protein, 96 aa, Artificial Sequence
  Synthetic Construct
GILNLNKMVKQVTGKMPILSYWPYGCHCGLGGRGQPKDATDWCCQTADCCYDHLKTQGCSIYKDNNKSSIHCMDLSQRYCLMAVFNVIYLENEDSE

SEQ ID NO: 13 - protein, 1 aa, Artificial Sequence
  Synthetic Construct
  VARIANT 1: Can be present in repeats of any integer
G

SEQ ID NO: 14 - protein, 2 aa, Artificial Sequence
  Synthetic Construct
  VARIANT (1)..(2): Can be present in repeats of any integer from 1 to 20
GS

SEQ ID NO: 15 - protein, 5 aa, Artificial Sequence
  Synthetic Construct
  VARIANT (1)..(5): Can be present in repeats of any integer from 1 to 8
GSGGS

SEQ ID NO: 16 - protein, 5 aa, Artificial Sequence
  Synthetic Construct
  VARIANT (1)..(5): Can be present in repeats of any integer from 1 to 8
GGGGS

SEQ ID NO: 17 - protein, 4 aa, Artificial Sequence
  Synthetic Construct
  VARIANT (1)..(4): Can be present in repeats of any integer from 1 to 8
GGGS

SEQ ID NO: 18 - protein, 15 aa, Artificial Sequence
  Synthetic Construct
GGGGSGGGGSGGGGS

SEQ ID NO: 19 - protein, 30 aa, Artificial Sequence
  Synthetic Construct
GGGGSGGGGSGGGGSGGGGSGGGGSGGGGS

SEQ ID NO: 20 - protein, 15 aa, Artificial Sequence
  Synthetic Construct
  VARIANT (1)..(15): Can be present in repeats of any integer from 1 to 3
GSTSGSGKPGSGEGS

SEQ ID NO: 21 - protein, 22 aa, Artificial Sequence
  Synthetic Construct
AEAAAKEAAAKEAAAKEAAAKA

SEQ ID NO: 22 - protein, 145 aa, Homo sapiens; identical to SEQ ID NO: 1

SEQ ID NO: 23 - protein, 100 aa, Artificial Sequence
  Synthetic Construct
ILNLNKMVKQVTGKMPILSYWPYGCHCGLGGRGQPKDATDWCCQTHDCCYDHLKTQGCSIYKDYYRYNFSQGNIHCSDKGSWCEQQLCACDKEVAFCLKR

SEQ ID NO: 24 - protein, 107 aa, Artificial Sequence
  Synthetic Construct
VWQFRKMIKCVIPGSDPFLEYNNYGCYCGLGGSGTPVDELDKCCQTHDNCYDQAKKLDSCKFLLDNPYTHTYSYSCSGSAITCSSKNKECEAFICNCDRNAAICFSK

SEQ ID NO: 25 - protein, 145 aa, Homo sapiens; identical to SEQ ID NO: 1

SEQ ID NO: 26 - protein, 144 aa, Artificial Sequence
  Synthetic Construct
MKTLLLLAVIMIFGLLQAHGNLVNFHRMIKLTTGKEAALSYGFYGCHCGVGGRGSPKDATDRCCVTHDCCYKRLEKRGCGTKFLSYKFSNSGSRITCAKQDSCRSQLCECDKAAATCFARNKTTYNKKYQYYSNKHCRGSTPRC

SEQ ID NO: 27 - protein, 129 aa, Artificial Sequence
  Synthetic Construct
PIQGGILNLNKMVKQVTGKMPILSYWPYGCHCGLGGRGQPKDATDWCCQTHDCCYDHLKTQGCSIYKDYYRYNFSQGNIHCSDKGSWCEQQLCACDKEVAFCLKRNLDTYQKRLRFYWRPHCRGQTPGC

SEQ ID NO: 28 - protein, 135 aa, Artificial Sequence
  Synthetic Construct
PTHSSFWQFQRRVKHITGRSAFFSYYGYGCYCGLGDKGIPVDDTDRHSPSSPSPYEKLKEFSCQPVLNSYQFHIVNGAVVCGCTLGPGASCHCRLKACECDKQSVHCFKESLPTYEKNFKQFSSQPRCGRHKPWC

SEQ ID NO: 29 - protein, 128 aa, Artificial Sequence
  Synthetic Construct
IQGGILNLNKMVKQVTGKMPILSYWPYGCHCGLGGRGQPKDATDWCCQTHDCCYDHLKTQGCSIYKDYYRYNFSQGNIHCSDKGSWCEQQLCACDKEVAFCLKRNLDTYQKRLRFYWRPHCRGQTPGC

SEQ ID NO: 30 - protein, 126 aa, Artificial Sequence
  Synthetic Construct
VTGNLVQFGVMIEKMTGKSALQYNDYGCYCGIGGSHWPVDQTDWCCHAHDCCYGRLEKLGCEPKLEKYLFSVSERGIFCAGRTTCQRLTCECDKRAALCFRRNLGTYNRKYAHYPNKLCTGPTPPC

SEQ ID NO: 31 - protein, 126 aa, Artificial Sequence
  Synthetic Construct
GGILNLNKMVKQVTGKMPILSYWPYGCHCGLGGRGQPKDATDWCCQTHDCCYDHLKTQGCSIYKDYYRYNFSQGNIHCSDKGSWCEQQLCACDKEVAFCLKRNLDTYQKRLRFYWRPHCRGQTPGC

SEQ ID NO: 32 - protein, 126 aa, Artificial Sequence
  Synthetic Construct
GSLLNLKAMVEAVTGRSAILSFVGYGCYCGLGGRGQPKDEVDWCCHAHDCCYQELFDQGCHPYVDHYDHTIENNTEIVCSDLNKTECDKQTCMCDKNMVLCLMNQTYREEYRGFLNVYCQGPTPNC

SEQ ID NO: 33 - protein, 145 aa, Homo sapiens; identical to SEQ ID NO: 1

SEQ ID NO: 34 - protein, 144 aa, Mus musculus
MRLALLCGLLLAGITATQGGLLNLNKMVTHMTGKKAFFSYWPYGCHCGLGGKGQPKDATDWCCQKHDCCYAHLKIDGCKSLTDNYKYSISQGTIQCSDNGSWCERQLCACDKEVALCLKQNLDSYNKRLRYYWRPRCKGKTPAC

SEQ ID NO: 35 - protein, 144 aa, Rattus rattus
MRLALLCGLLLAGITATQGGLLNLNKMVNHMTGKKAFFSYWPYGCHCGFGGKGQPKDATDWCCQKHDCCYAHLKIDGCKSLTDNYKYSISEGVIQCSDQGSWCERQLCACDKEVALCLKQNLESYNKRLRYYWRPRCKGQTPTC

SEQ ID NO: 36 - protein, 145 aa, Macaca mulatta
MQLALLCGLVVMAGVIPIQGGILNLNKMVKQVTGKMPLFFYWPYGCYCGPGGRGQPKDATDWCCQTHDCCYDHLKTHGCCIHTDHYRYSFSHGDIHCSDKGSWCEQQLCACDKEVAFCLKRNLDTYKKRLRFYWRPRCQGQTPGC

SEQ ID NO: 37 - protein, 145 aa, Pan troglodytes
MELALLCGLVVMAGVIPIQGGILNLNKMVKQVTGKMPILFYWPYGCHCGLGGRGQPKDATDWCCQTHDCCYDHLKTQGCGIYKDYYRYNFSQGNIHCSDKGSWCEQQLCACDKEVAFCLKRNLDTYQKRLRFYWRPHCRGQTPGC

(19) United States
(12) Patent Application Publication
(10) Pub. No.: US 2023/0075779 A1
(43) Pub. Date: Mar. 9, 2023

(54) METHODS AND COMPOSITIONS FOR TREATING CANCER OR VIRAL INFECTION WITH A PLA2G2D ANTAGONIST

(71) Applicant: Apeximmune Therapeutics Inc., South San Francisco, CA (US)

(72) Inventors: Li-Fen LEE, South San Francisco, CA (US); Kan LU, South San Francisco, CA (US); Jessica YU, South San Francisco, CA (US); Sheng-Tien LI, Taipei City (TW); Julie HUANG, South San Francisco, CA (US); Katharine YU, Walnut Creek, CA (US)

(73) Assignee: Apeximmune Therapeutics Inc., South San Francisco, CA (US)

(21) Appl. No.: 17/796,148

(22) PCT Filed: Jan. 29, 2021
(86) PCT No.: PCT/US2021/015921

§ 371 Date: Jul. 28, 2022

Related U.S. Application Data
(60) Provisional application No. 62/968,060, filed on Jan. 30, 2020.

(51) Int. Cl. C12N 9/18; A61P 35/00; C07K 16/40; A61K 38/46; A61K 45/06

(52) U.S. Cl. CPC C12N 9/18; A61P 35/00; C07K 16/40; A61K 38/465; A61K 45/06; C12Y 301/01004; A61K 2039/505

(57) ABSTRACT

The present application provides methods of treating a disease (such as cancer or infectious disease) that involves an antagonist that targets PLA2G2D signaling pathway (such as an antagonist that targets PLA2G2D. The present application also provides non-naturally occurring PLA2G2D polypeptides.

### CROSS REFERENCE TO RELATED APPLICATIONS

This application claims priority benefit of U.S. Provisional Application 62/968,060 filed Jan. 30, 2020, the contents of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 196882000140SEQLIST.TXT, date recorded: Jan. 29, 2021, size: 34 KB).

### FIELD OF THE APPLICATION

The present invention relates to methods and compositions for treating a disease or condition that involve an antagonist targeting PLA2G2D signaling pathway.

### BACKGROUND

The mechanisms by which the immune system responds to an infection or disease depend on a complex interplay between the elements of innate and adaptive immunity. Unwanted suppression of immune response stays as a major hurdle for patients' own immune system or promising treatments such as immunotherapies to fight against the disease or infection. For example, a fundamental problem in the effort to treat patients with an immunotherapy, is that the tumor-bearing state is associated with immunosuppressive mechanisms derived from both the tumor and the host's disturbed immune system, thereby preventing the therapy to achieve the ideal efficacy.

The disclosures of all publications, patents, patent applications and published patent applications referred to herein are hereby incorporated herein by reference in their entirety for all purposes.

### BRIEF SUMMARY OF THE APPLICATION

The present application provides methods of treating diseases or conditions (such as cancer or viral infection).

The present application in one aspect provides methods of treating a cancer or viral infection in an individual, comprising administering into the individual an effective amount of an antagonist targeting PLA2G2D signaling pathway. In some embodiments, the antagonist is an antagonist targeting PLA2G2D. In some embodiments, the PLA2G2D is a human PLA2G2D. In some embodiments, the antagonist decreases enzymatic activity level of PLA2G2D. In some embodiments, the antagonist targeting PLA2G2D signaling pathway blocks a catalytic site on PLA2G2D. In some embodiments, the antagonist targets the H67 catalytic site on a human PLA2G2D according to SEQ ID NO: 1 or 5.

In some embodiments, the antagonist comprises a siRNA, a miRNA, an antisense RNA, or a gene editing system.

In some embodiments, the antagonist comprises an agent that inhibits PLA2G2D (such as an agent that blocks the binding of PLA2G2D to an immune cell or an agent that inhibits the activity of PLA2G2D). In some embodiments, the immune cell is a T cell.

In some embodiments, the antagonist comprises an anti-PLA2G2D antibody. In some embodiments, the anti-PLA2G2D antibody is a monoclonal antibody. In some embodiments, the antagonist is a fusion protein or immunoconjugate comprising an anti-PLA2G2D antibody moiety and a second moiety. In some embodiments, the second moiety comprises a cytokine.

In some embodiments, the antagonist comprises an inhibitory PLA2G2D polypeptide that blocks the binding of PLA2G2D to an immune cell.

In some embodiments, the inhibitory PLA2G2D polypeptide binds to the immune cell with a greater affinity than for PLA2G2D. In some embodiments, the immune cells is a T cell. In some embodiments, the inhibitory polypeptide further comprises a stabilizing domain.

In some embodiments, the stabilizing domain is an Fc domain. In some embodiments, the inhibitory PLA2G2D polypeptide has a length of about 50 to about 200 amino acids. In some embodiments, the inhibitory PLA2G2D polypeptide has a mutation at the position corresponding to histidine at position 67 (H67) according to SEQ ID NO: 1 or 5. In some embodiments, the inhibitory PLA2G2D polypeptide comprises an amino acid sequence of SEQ ID NO: 3, 4, 7, or 8.

In some embodiments according to any one of the methods described above, the disease or condition is a cancer. In some embodiments, the cancer is a solid tumor. In some embodiments, the cancer is an advanced or malignant tumor. In some embodiments, the cancer has an increased expression level of PLA2G2D. In some embodiments, the cancer is selected from the group consisting of lung cancer, breast cancer, liver cancer, gastric cancer, cervical cancer, endometrial cancer, thyroid cancer, colorectal cancer, head and neck cancer, pancreatic cancer, renal cancer, prostate cancer, urothelial cancer, testis cancer, ovarian cancer and melanoma. In some embodiments, the disease or condition is a viral infection. In some embodiments, the infection site has an increased expression level of PLA2G2D.

In some embodiments according to any one of the methods described above, the method further comprises administering a second agent. In some embodiments, the second agent is selected from the group consisting of a chemotherapeutic agent, an immunomodulator, an anti-angiogenesis agent, a growth inhibitory agent, and an antineoplastic agent. In some embodiments, the second agent is an immunomodulator. In some embodiments, the immunomodulator is an immune checkpoint inhibitor. In some embodiments, the immune checkpoint inhibitor specifically target PD-L1, PD-L2, CTLA4, PD-L2, PD-1, CD47, TIGIT, GITR, TIM3, LAG3, CD27, 4-1BB, or B7H4. In some embodiments, the second agent comprises a cell comprising a chimeric antigen receptor that specifically binds to a tumor antigen. In some embodiments, the antagonist and the second agent is administered simultaneously or concurrently. In some embodiments, the antagonist and the second agent is administered sequentially. In some embodiments, the antagonist and/or the second agent is administered parentally. In some embodiments, the antagonist is administered to the cancer tissue or infection site directly.

In some embodiments according to any of the methods described above, the antagonist is administered at a dose of about 0.001 μg/kg to about 100 mg/kg.

In some embodiments according to any of the methods described above, the individual has an increased number of immune cells in the cancer tissue or at the infection site after administration of the antagonist. In some embodiments, the immune cells are T cells. In some embodiment, the T cells are activated T cells. In some embodiments, the number of immune cells in the cancer tissue or at the infection site is increased by at least about 5% (such as at least about 5%, 10%, 15%, 20%, 25%, 30%, 40%, 50%, 60%, 70%, 80%, 90%, 1-fold, 2-fold, 3-fold, 4-fold, or 5-fold) after administration of the antagonist.

In some embodiments according to any of the methods described above, immune cells in the cancer tissue or at the infection site produce an increased level of a cytokine after administration of the antagonist. In some embodiments, the cytokine is IFNγ and/or IL-2. In some embodiments, the level of the cytokine is increased by at least about 5% (such as at least about 5%, 10%, 15%, 20%, 25%, 30%, 40%, 50%, 60%, 70%, 80%, 90%, 1-fold, 2-fold, 3-fold, 4-fold, or 5-fold) after administration of the antagonist.

### BRIEF DESCRIPTION OF THE DRAWINGS

FIGS. 1A-1D shows that PLA2G2D is highly differentially expressed in human (FIG. 1A) lung adenocarcinoma, (FIG. 1B) triple negative breast cancer, (FIG. 1C) live hepatocellular carcinoma, and (FIG. 1D) stomach adenocarcinoma. Relative expression and significance of PD-1, CTLA-4 and TIGIT are also indicated.

FIG. 2A shows that soluble human PLA2G2D-Fc protein dose-dependently suppresses PBMC derived CD4+ and CD8+ T cell proliferation in the presence of anti-CD3 and anti-CD28 stimulation.

FIG. 2B shows a quantitative graph of the effect of human PLA2G2D-Fc protein on PBMC-derived CD4+ and CD8+ T cell proliferation.

FIGS. 3A-3C show that soluble PLA2G2D protein dose-dependently suppresses T cell proliferation in different PBMC donors in the presence of anti-CD3 and anti-CD28 stimulation. T cell CFSE proliferation as analyzed by flow cytometry is shown on the left, and quantitative representations of percent CD4+ and CD8+ T cell proliferation are shown on the right.

FIGS. 4A-4B show that soluble PLA2G2D protein dose-dependently suppresses IFN J and IL-2 levels in correlation with suppression of T cell proliferation across different donors.

FIG. 5 shows that immobilized PLA2G2D protein dose-dependently suppresses T cell proliferation in PBMC culture in the presence of anti-CD3 and anti-CD28 stimulation. T cell CFSE proliferation as analyzed by flow cytometry is shown on the left, and quantitative representations of percent CD4+ and CD8+ T cell proliferation are shown on the right.

FIG. 6 shows that immobilized PLA2G2D protein dose-dependently suppresses proliferation of isolated T cell cultures in the presence of anti-CD3 and anti-CD28 stimulation. T cell CFSE proliferation as analyzed by flow cytometry is shown on the left, and quantitative representations of percent CD4+ and CD8+ T cell proliferation are shown on the right.

FIG. 7A shows structural and functional features of interest of the human PLA2G2D protein (SEQ ID NO: 22) including its signal peptide (the first 20 amino acids), calcium binding sites, catalytic sites, N-linked glycosylation site, and active site. A H67Q catalytic site mutant was generated to create an enzymatic-deficient PLA2G2D protein.

FIGS. 7B-7C show that a H47Q-PLA2G2D catalytic mutant retains most of the immune suppressive functions on CD4+(7B) and CD8+(7C) T cells.

FIG. 8 shows that a general inhibitor for various PLA2 small molecule Inhibitor, LY315920, does not rescue immune suppression by PLA2G2D.

FIGS. 9A-9C show that human PLA2G2D-Fc preferentially binds activated CD4+ and CD8+ T cells in different donor T cells compared with control-Fc protein. PLA2G2D binds unstimulated T cells to a small degree, but binding is dramatically increased upon T cell stimulation. FIG. 9C shows a quantitative representation of PLA2G2D-Fc binding to stimulated T cells.

FIG. 10A shows a predicted automated 3D structure based upon Swiss-model.

FIG. 10B shows sequence homology of different PLA2 Group 2 family members to PLA2G2D. SEQ ID NOs from top to bottom are SEQ ID NOs 22-32.

FIG. 10C shows sequence homology of PLA2G2D from different species vs. human. Human (SEQ ID NO: 33), Mouse (SEQ ID NO: 34), Rat (SEQ ID NO: 35), Rhesus (SEQ ID NO: 36), and Chimp (SEQ ID NO: 37).

FIG. 11A shows that the mean tumor volume of syngeneic subcutaneous MC38 colon adenocarcinoma tumors implanted in PLA2G2D knockout (KO) mice (n=16) is significantly reduced compared to wild type (WT) C57BL6 mice (n=16). FIG. 11B shows the tumor growth kinetics of individual animals from each group.

FIG. 11C shows that the mean tumor volume of syngeneic subcutaneous B16F10 melanoma tumors implanted in PLA2G2D knockout (KO) mice (n=16) is significantly reduced compared to wild type (WT) C57BL6 mice (n=16). FIG. 1I D shows the tumor growth kinetics of individual animals from each group.

FIG. 11E shows that the mean tumor volume of syngeneic subcutaneous E.G7-OVA T cell lymphoma tumors implanted in PLA2G2D knockout (KO) mice (n=16) is significantly reduced compared to wild type (WT) C57BL6 mice (n=16). FIG. 11F shows the tumor growth kinetics of individual animals from each group.

FIG. 12A shows that the mean fluorescence intensity (MFI) of PLA2G2D-Fc binding to activated T cells in PBMC culture can be blocked by anti-PLA2G2D antibodies.

FIGS. 12B-12C show that PLA2G2D-Fc mediated suppression of IL-2 and IFNγ levels in T cell-activated PBMC cultures can be reversed by the addition of function-blocking anti-PLA2G2D antibodies.

### DETAILED DESCRIPTION OF THE APPLICATION

The present application in one aspect provides methods of treating a disease or condition (such as cancer or infectious disease) that involves administering an antagonist targeting PLA2G2D signaling pathway. In some embodiments, the antagonist comprises an agent binding to PLA2G2D (such as an agent comprising an anti-PLA2G2D antibody moiety). In some embodiments, the antagonist comprises an inhibitory PLA2G2D polypeptide. In some embodiments, the antagonist comprises a nucleic acid agent targeting PLA2G2D (such as a siRNA or antisense RNA). In some embodiments, the antagonist comprises an agent that inhibits PLA2G2D enzymatic activity. The present application in another aspect provides non-naturally occurring poly peptides such as the inhibitory PLA2G2D polypeptides that can be used for treatment.

The present application is at least partly based upon the striking finding that PLA2G2D plays a crucial role in suppressing a key player in immune system, T cells. Specifically, it was found that PLA2G2D was expressed 56 times higher in CD8+ high tumors than CD8+ low tumors. As shown in Examples in more details, PLA2G2D can both directly and indirectly (e.g., via cross-linking with antigen-presenting cells) inhibit both CD4+ and CD8+ T cells' proliferation, activation and/or cytokine production, which can lead to a significant level of suppression of immune response in diseases such as cancer, especially in the diseased tissue such as a cancer tissue. It was also found that PLA2G2D's enzymatic activity only partially contributes to its role in suppressing immune response, and that PLA2G2D can directly bind to T cells, especially activated T cells. Without being bound to the theory, it is believed that at least part of the suppressive function of PLA2G2D on T cells is exerted by the binding of PLA2G2D to the cells. The present application has for the first time promising novel methods of using an antagonist that targets PLA2G2D pathway (such as an agent comprising an anti-PLA2G2D antibody moiety or an inhibitory PLA2G2D polypeptide) to treat a disease or condition in which the immune response is suppressed, including, for example, cancer and infectious disease (such as viral infectious disease).

### I. DEFINITIONS

Unless specifically indicated otherwise, all technical and scientific terms used herein have the same meaning as commonly understood by those of ordinary skill in the art to which this application belongs. In addition, any method or material similar or equivalent to a method or material described herein can be used in the practice of the present application.

For purposes of the present application, the following terms are defined.

It is understood that embodiments of the application described terms of “comprising” herein include “consisting” and/or “consisting essentially of” embodiments.

As used herein the term “wild type” is a term of the art understood by skilled persons and means the typical form of an organism, strain, gene or characteristic as it occurs in nature as distinguished from mutant or variant forms.

As used herein the term “variant” should be taken to mean the exhibition of qualities that have a pattern that deviates from what occurs in nature.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “therapeutic agent”, “therapeutic capable agent” or “treatment agent” are used interchangeably and refer to a molecule or compound that confers some beneficial effect upon administration to an individual. The beneficial effect includes enablement of diagnostic determinations; amelioration of a disease, symptom, disorder, or pathological condition: reducing or preventing the onset of a disease, symptom, disorder or condition, and generally counteracting a disease, symptom, disorder or pathological condition.

The term “antibody” is used in its broadest sense and encompasses various antibody structures, including but not limited to monoclonal antibodies, polyclonal antibodies, multispecific antibodies (e.g., bispecific antibodies), humanized antibodies, chimeric antibodies, full-length antibodies and antigen-binding fragments thereof, so long as they exhibit the desired antigen-binding activity. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004, each of which are incorporated herein by reference in their entirety for all purposes).

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “epitope” as used herein refers to the specific group of atoms or amino acids on an antigen to which an antibody or diabody binds. Two antibodies or antibody moieties may bind the same epitope within an antigen if they exhibit competitive binding for the antigen.

The terms “polypeptide” or “peptide” are used herein to encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamlation, ADP-ribosylation, pegylation, biotinylation, etc.).

As use herein, the terms “specifically binds,” “specifically recognizing,” and “is specific for” refer to measurable and reproducible interactions, such as binding between a target and an antibody (such as a diabody). In certain embodiments, specific binding is determinative of the presence of the target in the presence of a heterogeneous population of molecules, including biological molecules (e.g., cell surface receptors). For example, an antibody that specifically recognizes a target (which can be an epitope) is an antibody (such as a diabody) that binds this target with greater affinity, avidity, more readily, and/or with greater duration than its bindings to other molecules. In some embodiments, the extent of binding of an antibody to an unrelated molecule is less than about 10% of the binding of the antibody to the target as measured. e.g., by a radioimmunoassay (RIA). In some embodiments, an antibody that specifically binds a target has a dissociation constant (KD) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, an antibody specifically binds an epitope on a protein that is conserved among the protein from different species. In some embodiments, specific binding can include, but does not require exclusive binding. Binding specificity of the antibody or antigen-binding domain can be determined experimentally by methods known in the art. Such methods comprise, but are not limited to Western blots, ELISA, RIA, ECL, IRMA, EIA, BIACORE™ and peptide scans.

As used herein, “the composition” or “compositions” includes and is applicable to compositions of the application. The application also provides pharmaceutical compositions comprising the components described herein.

As used herein, “treatment” or “treating” is an approach for obtaining beneficial or desired results including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. The methods of the application contemplate any one or more of these aspects of treatment. The benefit to an individual to be treated is either statistically significant or at least perceptible to the patient or to the physician.

The term “effective amount” used herein refers to an amount of an agent or composition sufficient to treat a specified state, disorder, condition, or disease such as ameliorate, palliate, lessen, and/or delay one or more of its symptoms (e.g., clinical or sub-clinical symptoms). For therapeutic use, beneficial or desired results include, e.g., decreasing one or more symptoms resulting from the disease (biochemical, histologic and/or behavioral), including its complications and intermediate pathological phenotypes presenting during development of the disease, increasing the quality of life of those suffering from the disease, decreasing the dose of other medications required to treat the disease, enhancing effect of another medication, delaying the progression of the disease, and/or prolonging survival of patients. In reference to a cancer, an effective amount comprises an amount sufficient to cause a cancer tissue to shrink and/or to decrease the growth rate of the cancer tissue or to prevent or delay other unwanted cell proliferation in the cancer. In some embodiments, an effective amount is an amount sufficient to delay development of a cancer. In some embodiments, an effective amount is an amount sufficient to prevent or delay recurrence. An effective amount can be administered in one or more administrations. In the case of cancer, the effective amount of the drug or composition may: (i) reduce the number of tumor cells: (ii) reduce the tumor size; (iii) inhibit, retard, slow to some extent and preferably stop a tumor cell infiltration into peripheral organs; (iv) inhibit (i.e., slow to some extent and preferably stop) tumor metastasis; (v) inhibit tumor growth: (vi) prevent or delay occurrence and/or recurrence of tumor, and/or (vii) relieve to some extent one or more of the symptoms associated with the cancer. Note that when a combination of active ingredients is administered, the effective amount of the combination may or may not include amounts of each ingredient that would have been effective if administered individually. The exact amount required will vary from individual to individual, depending on the species, age, and general condition of the individual, the severity of the condition being treated, the particular drug or drugs employed, the mode of administration, and the like.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, by “pharmaceutically acceptable” or “pharmacologically compatible” is meant a material that is not biologically or otherwise undesirable. e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration or other state/federal government or listed in the U.S.

Pharmacopeia or other generally recognized pharmacopeia for use in mammals, and more particularly in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin, incorporated by reference in its entirety for all purposes.

The term “tumor” refers to or describes the physiological condition in mammals that is typically characterized by unregulated cell growth and includes benign or malignant abnormal growth of tissue. The term “tumor” includes cancer.

The terms “individual,” “subject,” and “patient” are used interchangeably herein to refer to a mammal, including, but not limited to, human, bovine, horse, feline, canine, rodent, or primate. In some embodiments, the individual is a human. In a preferred embodiment, the individual is a human.

Reference to “about” a value or parameter herein includes (and describes) variations that are directed to that value or parameter per se. For example, description referring to “about X” includes description of “X”. In certain embodiments, a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The term “about X-Y” used herein has the same meaning as “about X to about Y.”

As used herein and in the appended claims, the singular forms “a,” “an.” “or,” and “the” include plural referents unless the context clearly dictates otherwise. Thus, for example, a reference to “a method” includes one or more methods, and/or steps of the type described herein and/or which will become apparent to those persons skilled in the art upon reading this disclosure. As is apparent to one skilled in the art, an individual assessed, selected for, and/or receiving treatment is an individual in need of such activities.

The practice of the present disclosure employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, N.Y.: Ausubel et al. eds. (2005) Current Protocols in Molecular Biology. John Wiley and Sons, Inc.: Hoboken, N.J.: Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken. N.J.; Coico et al. eds. (2005) Current Protocols in Microbiology, John Wiley and Sons, Inc.: Hoboken, N.J.: Coligan et al. eds. (2005) Current Protocols in Protein Science, John Wiley and Sons, Inc.: Hoboken. N.J.; and Enna et al. eds. (2005) Current Protocols in Pharmacology, John Wiley and Sons, Inc.: Hoboken, N.J. Additional techniques are explained, e.g., in U.S. Pat. No. 7,912,698 and U.S. Patent Appl. Pub. Nos. 2011/0202322 and 2011/0307437, each of which is incorporated by reference in their entirety for all purposes.

The terms and expressions which have been employed are used as terms of description and not of limitation, and use of such terms and expressions do not exclude any equivalents of the features shown and described or portions thereof, and various modifications are possible within the scope of the technology claimed.

### II. METHODS OF TREATMENT

The present application in one aspect provides methods of treating a disease or condition (such as cancer or infectious disease) in an individual, comprising administering to the individual an effective amount of an antagonist that targets PLA2G2D signaling pathway. In some embodiments, the antagonist comprises an agent binding to PLA2G2D (such as an agent comprising an anti-PLA2G2D antibody moiety). In some embodiments, the antagonist comprises an inhibitory PLA2G2D polypeptide. In some embodiments, the antagonist comprises a nucleic acid agent targeting PLA2G2D (such as a siRNA or antisense RNA). In some embodiments, the antagonist comprises an agent that inhibits PLA2G2D enzymatic activity.

In some embodiments, there is provided a method of treating a cancer (such as a solid tumor, a colon cancer, melanoma, or a T cell lymphoma) in an individual, comprising administering into the individual an effective amount of an antagonist that comprises an agent that inhibits PLA2G2D (such as an agent that blocks the binding of PLA2G2D to an immune cell or an agent that inhibits the activity of PLA2G2D). In some embodiments, the immune cell is a T cell (such as an activated T cell, such as activated CD4+ T cells or CD8+ T cells). In some embodiments, the antagonist comprises an anti-PLA2G2D antibody. In some embodiments, the anti-PLA2G2D antibody is a monoclonal antibody. In some embodiments, the antagonist is a fusion protein or immunoconjugate comprising an anti-PLA2G2D antibody moiety and a second moiety, such as a second moiety comprising a cytokine (such as a pro-inflammatory cytokine). In some embodiment, the PLA2G2D is a human PLA2G2D. In some embodiments, the cancer tissue has an increased expression level of PLA2G2D as compared to a reference tissue (such as a corresponding tissue in a healthy individual). In some embodiments, the cancer is an advanced or malignant tumor. In some embodiments, the cancer is selected from the group consisting of lung cancer, breast cancer, liver cancer, gastric cancer, cervical cancer, endometrial cancer, thyroid cancer, colorectal cancer, head and neck cancer, pancreatic cancer, renal cancer, prostate cancer, urothelial cancer, testis cancer, ovarian cancer and melanoma. In some embodiments, the method further comprises administering a second agent. In some embodiments, the second agent is selected from the group consisting of a chemotherapeutic agent, an immunomodulator, an anti-angiogenesis agent, a growth inhibitory agent, and an antineoplastic agent. In some embodiments, the second agent is an immunomodulator. In some embodiments, the immunomodulator is an immune checkpoint inhibitor. In some embodiments, the immune checkpoint inhibitor specifically target PD-L1, PD-L2, CTLA4, PD-L2, PD-1, CD47, TIGIT, GITR, TIM3, LAG3, CD27, 4-1BB, or B7H4. In some embodiments, the second agent comprises a cell comprising a chimeric antigen receptor that specifically binds to a tumor antigen. In some embodiments, the antagonist and the second agent is administered simultaneously or concurrently. In some embodiments, the antagonist and the second agent is administered sequentially. In some embodiments, the antagonist and/or the second agent is administered parentally. In some embodiments, the antagonist is administered to diseased tissue directly.

In some embodiments, there is provided a method of treating an infectious disease (such as a viral infectious disease) in an individual, comprising administering into the individual an effective amount of an antagonist that comprises an agent that inhibits PLA2G2D (such as an agent that blocks the binding of PLA2G2D to an immune cell or an agent that inhibits the activity of PLA2G2D). In some embodiments, the immune cell is a T cell (such as an activated T cell, such as activated CD4+ T cells or CD8+ T cells). In some embodiments, the antagonist comprises an anti-PLA2G2D antibody. In some embodiments, the anti-PLA2G2D antibody is a monoclonal antibody. In some embodiments, the antagonist is a fusion protein or immunoconjugate comprising an anti-PLA2G2D antibody moiety and a second moiety. In some embodiments, the second moiety comprises a cytokine (such as a pro-inflammatory cytokine). In some embodiment, the PLA2G2D is a human PLA2G2D. In some embodiments, the infection site has an increased expression level of PLA2G2D as compared to a reference tissue (such as a corresponding tissue in a healthy individual). In some embodiments, the method further comprises administering a second agent. In some embodiments, the second agent comprises an immune therapy. In some embodiments, the antagonist and the second agent is administered simultaneously or concurrently. In some embodiments, the antagonist and the second agent is administered sequentially. In some embodiments, the antagonist and/or the second agent is administered parentally. In some embodiments, the antagonist is administered to diseased tissue directly.

In some embodiments, there is provided a method of treating a cancer (such as a solid tumor, a colon cancer, melanoma, or a T cell lymphoma) in an individual, comprising administering into the individual an effective amount of an antagonist comprises an inhibitory PLA2G2D polypeptide that inhibits PLA2G2D (such as an inhibitory polypeptide that blocks the binding of PLA2G2D to an immune cell). In some embodiments, the inhibitory PLA2G2D polypeptide binds to the immune cell with a greater affinity than for PLA2G2D (such as a wildtype PLA2G2D). In some embodiments, the immune cell is a T cell (such as an activated T cell, such as activated CD4+ T cells or CD8+ T cells). In some embodiments, the inhibitory PLA2G2D polypeptide further comprises a stabilizing domain. In some embodiments, the stabilizing domain is an Fc domain. In some embodiments, the inhibitory PLA2G2D polypeptide has a length of about 50 to about 200 amino acids. In some embodiments, the inhibitory PLA2G2D polypeptide has a) a mutation at the position corresponding to histidine at position 67 (H67) according to SEQ ID NO: 1 or 5 or b) a mutation at the position corresponding to glycine at position 80 (G80) according to SEQ ID NO: 5. In some embodiments, the inhibitory PLA2G2D polypeptide comprises an amino acid sequence selected from the group consisting of SEQ ID NOs: 3, 4, and 7-12 or a variant thereof. In some embodiment, the PLA2G2D is a human PLA2G2D. In some embodiments, the cancer tissue has an increased expression level of PLA2G2D as compared to a reference tissue (such as a corresponding tissue in a healthy individual). In some embodiments, the cancer is an advanced or malignant tumor. In some embodiments, the cancer is selected from the group consisting of lung cancer, breast cancer, liver cancer, gastric cancer, cervical cancer, endometrial cancer, thyroid cancer, colorectal cancer, head and neck cancer, pancreatic cancer, renal cancer, prostate cancer, urothelial cancer, testis cancer, ovarian cancer and melanoma. In some embodiments, the method further comprises administering a second agent. In some embodiments, the second agent is selected from the group consisting of a chemotherapeutic agent, an immunomodulator, an anti-angiogenesis agent, a growth inhibitory agent, and an antineoplastic agent. In some embodiments, the second agent is an immunomodulator. In some embodiments, the immunomodulator is an immune checkpoint inhibitor. In some embodiments, the immune checkpoint inhibitor specifically target PD-L1, PD-L2, CTLA4, PD-L2, PD-1, CD47, TIGIT, GITR, TIM3, LAG3, CD27, 4-1BB, or B7H4. In some embodiments, the second agent comprises a cell comprising a chimeric antigen receptor that specifically binds to a tumor antigen. In some embodiments, the antagonist and the second agent is administered simultaneously or concurrently. In some embodiments, the antagonist and the second agent is administered sequentially. In some embodiments, the antagonist and/or the second agent is administered parentally. In some embodiments, the antagonist is administered to diseased tissue directly.

In some embodiments, there is provided a method of treating an infectious disease (such as a viral infectious disease) in an individual, comprising administering into the individual an effective amount of an antagonist comprises an inhibitory PLA2G2D polypeptide that blocks the binding of PLA2G2D to an immune cell. In some embodiments, the inhibitory PLA2G2D polypeptide binds to the immune cell with a greater affinity than for PLA2G2D (such as a wildtype PLA2G2D). In some embodiments, the immune cell is a T cell (such as an activated T cell, such as activated CD4+ T cells or CD8+ T cells). In some embodiments, the inhibitory PLA2G2D polypeptide further comprises a stabilizing domain. In some embodiments, the stabilizing domain is an Fc domain. In some embodiments, the inhibitory PLA2G2D polypeptide has a length of about 50 to about 200 amino acids. In some embodiments, the inhibitory PLA2G2D polypeptide has a) a mutation at the position corresponding to histidine at position 67 (H67) according to SEQ ID NO: 1 or 5 or b) a mutation at the position corresponding to glycine at position 80 (G80) according to SEQ ID NO: 5. In some embodiments, the inhibitory PLA2G2D polypeptide comprises an amino acid sequence selected from the group consisting of SEQ ID NOs: 3, 4, and 7-12 or a variant thereof. In some embodiment, the PLA2G2D is a human PLA2G2D. In some embodiments, the infection site has an increased expression level of PLA2G2D as compared to a reference tissue (such as a corresponding tissue in a healthy individual). In some embodiments, the method further comprises administering a second agent. In some embodiments, the second agent comprises an immune therapy. In some embodiments, the antagonist and the second agent is administered simultaneously or concurrently. In some embodiments, the antagonist and the second agent is administered sequentially. In some embodiments, the antagonist and/or the second agent is administered parentally. In some embodiments, the antagonist is administered to diseased tissue directly.

In some embodiments, there is provided a method of treating a cancer (such as a solid tumor, a colon cancer, melanoma, or a T cell lymphoma) in an individual, comprising administering into the individual an effective amount of an antagonist that comprises a nucleic acid agent that inhibits the expression of PLA2G2D. In some embodiments, the nucleic acid agent comprises a siRNA, a miRNA, or an antisense RNA. In some embodiment, the PLA2G2D is a human PLA2G2D. In some embodiments, the cancer tissue has an increased expression level of PLA2G2D as compared to a reference tissue (such as a corresponding tissue in a healthy individual). In some embodiments, the cancer is an advanced or malignant tumor. In some embodiments, the cancer is selected from the group consisting of lung cancer, breast cancer, liver cancer, gastric cancer, cervical cancer, endometrial cancer, thyroid cancer, colorectal cancer, head and neck cancer, pancreatic cancer, renal cancer, prostate cancer, urothelial cancer, testis cancer, ovarian cancer and melanoma. In some embodiments, the method further comprises administering a second agent. In some embodiments, the second agent is selected from the group consisting of a chemotherapeutic agent, an immunomodulator, an anti-angiogenesis agent, a growth inhibitory agent, and an antineoplastic agent. In some embodiments, the second agent is an immunomodulator. In some embodiments, the immunomodulator is an immune checkpoint inhibitor. In some embodiments, the immune checkpoint inhibitor specifically target PD-L1, PD-L2, CTLA4, PD-L2, PD-1, CD47, TIGIT, GITR, TIM3, LAG3, CD27, 4-1BB, or B7H4.

In some embodiments, the second agent comprises a cell comprising a chimeric antigen receptor that specifically binds to a tumor antigen. In some embodiments, the antagonist and the second agent is administered simultaneously or concurrently. In some embodiments, the antagonist and the second agent is administered sequentially. In some embodiments, the antagonist and/or the second agent is administered parentally. In some embodiments, the antagonist is administered to diseased tissue directly.

In some embodiments, there is provided a method of treating a cancer (such as a solid tumor, a colon cancer, melanoma, or a T cell lymphoma) in an individual, comprising administering into the individual an effective amount of an antagonist that comprises a nucleic acid agent that inhibits the expression of PLA2G2D, wherein the individual has high T cell infiltration in cancer tissue. In some embodiments, the high T cell infiltration comprises a high number, percentage or density of T cells (e.g., CD3 T cells, CD4 T cells, CD8 T cells, activated T cells, activated CD4 T cells, activated CD8 T cells) in the cancer tissue. In some embodiments, the high T cell infiltration is present when the number of the T cells in the cancer is at least about 5%, 10%, 15%, 20%, 25%, 30%, 40%, 50%, 60%, 70%, 80%, 90%, or 95% more than the number of the corresponding T cells in a reference tissue. In some embodiments, the high T cell infiltration is present when the number of the T cells in the cancer is at least about 1-fold, 2-fold, 3-fold, 4-fold, 5-fold, 6-fold, 7-fold, 8-fold, 9-fold, or 10-fold more than the number of the corresponding T cells in a reference tissue. In some embodiments, the reference tissue is the corresponding tissue in a healthy individual. In some embodiments, the number of the corresponding T cells in a reference tissue is the average number of the corresponding T cells in the same tissue in a group of individuals (such as 10, 30, 50, 100 individuals) with same or similar cancer. In some embodiments, the reference tissue is the corresponding tissue in an individual who also has a cancer but has a less suppressed immune response in the cancer tissue as indicated by a biomarker. Examples of biomarker indicative of immunosuppressive tumor microenvironment (TME) includes: a) a high number, percentage and/or density of M2 macrophages (e.g., CD68+CD163+ cells) in the tissue; b) a high expression level of an immune checkpoint agent (e.g., PD-1 or PD-L1). Methods assessing and evaluating these biomarkers are known. See e.g., Hensler et al., Journal for ImmunoTherapy of Cancer 2020; 8:e000979: Chen et al., J Biomed Sci 26, 78 (2019); Teng et al., Cancer Res. 2015 Jun. 1; 75(11): 2139-2145. In some embodiments, the nucleic acid agent comprises a siRNA, a miRNA, or an antisense RNA. In some embodiment, the PLA2G2D is a human PLA2G2D. In some embodiments, the cancer tissue has an increased expression level of PLA2G2D as compared to a reference tissue (such as a corresponding tissue in a healthy individual). In some embodiments, the cancer is an advanced or malignant tumor. In some embodiments, the cancer is selected from the group consisting of lung cancer, breast cancer, liver cancer, gastric cancer, cervical cancer, endometrial cancer, thyroid cancer, colorectal cancer, head and neck cancer, pancreatic cancer, renal cancer, prostate cancer, urothelial cancer, testis cancer, ovarian cancer and melanoma. In some embodiments, the method further comprises administering a second agent. In some embodiments, the second agent is selected from the group consisting of a chemotherapeutic agent, an immunomodulator, an anti-angiogenesis agent, a growth inhibitory agent, and an antineoplastic agent. In some embodiments, the second agent is an immunomodulator. In some embodiments, the immunomodulator is an immune checkpoint inhibitor. In some embodiments, the immune checkpoint inhibitor specifically target PD-L1, PD-L2, CTLA4, PD-L2, PD-1, CD47, TIGIT, GITR, TIM3, LAG3, CD27, 4-1BB, or B7H4. In some embodiments, the second agent comprises a cell comprising a chimeric antigen receptor that specifically binds to a tumor antigen. In some embodiments, the antagonist and the second agent is administered simultaneously or concurrently. In some embodiments, the antagonist and the second agent is administered sequentially. In some embodiments, the antagonist and/or the second agent is administered parentally. In some embodiments, the antagonist is administered to diseased tissue directly.

In some embodiments, there is provided a method of treating a cancer (such as a solid tumor, a colon cancer, melanoma, or a T cell lymphoma) in an individual, comprising administering into the individual an effective amount of an antagonist that comprises a nucleic acid agent that inhibits the expression of PLA2G2D, wherein the individual has a high expression level of PLA2G2D in the cancer tissue. In some embodiments, the cancer tissue has a high expression level of PLA2G2D when the expression level of PLA2G2D (e.g., assessed by immunohistochemistry) is at least about 5%, 10%, 15%, 20%, 25%, 30%, 40%, 50%, 60%, 70%, 80%, 90%, or 95% higher than the expression level of PLA2G2D in a reference tissue. In some embodiments, the cancer tissue has a high expression level of PLA2G2D when the expression level of PLA2G2D (e.g., assessed by immunohistochemistry) is at least about 1-fold, 2-fold, 3-fold, 4-fold, 5-fold, 6-fold, 7-fold, 8-fold, 9-fold, 10-fold, 20-fold, 30-fold, 40-fold, or 50-fold higher than the expression level of PLA2G2D in a reference tissue. In some embodiments, the reference tissue is the corresponding tissue in a healthy individual. In some embodiments, the expression level of PLA2G2D in a reference tissue is the average expression level of PLA2G2D in the same tissue in a group of individuals (such as 10, 30, 50, 100 individuals) with same or similar cancer. In some embodiments, the reference tissue is the corresponding tissue in an individual who also has a cancer but has a less suppressed immune response in the cancer tissue as indicated by a biomarker (such as high M2 macrophages, or high expression of an immune checkpoint agent such as PD-1 or PD-L1). In some embodiments, the nucleic acid agent comprises a siRNA, a miRNA, or an antisense RNA. In some embodiment, the PLA2G2D is a human PLA2G2D. In some embodiments, the cancer tissue has an increased expression level of PLA2G2D as compared to a reference tissue (such as a corresponding tissue in a healthy individual). In some embodiments, the cancer is an advanced or malignant tumor. In some embodiments, the cancer is selected from the group consisting of lung cancer, breast cancer, liver cancer, gastric cancer, cervical cancer, endometrial cancer, thyroid cancer, colorectal cancer, head and neck cancer, pancreatic cancer, renal cancer, prostate cancer, urothelial cancer, testis cancer, ovarian cancer and melanoma. In some embodiments, the method further comprises administering a second agent. In some embodiments, the second agent is selected from the group consisting of a chemotherapeutic agent, an immunomodulator, an anti-angiogenesis agent, a growth inhibitory agent, and an antineoplastic agent. In some embodiments, the second agent is an immunomodulator. In some embodiments, the immunomodulator is an immune checkpoint inhibitor. In some embodiments, the immune checkpoint inhibitor specifically target PD-L1, PD-L2, CTLA4, PD-L2, PD-1, CD47, TIGIT, GITR, TIM3, LAG3, CD27, 4-1BB, or B7H4. In some embodiments, the second agent comprises a cell comprising a chimeric antigen receptor that specifically binds to a tumor antigen. In some embodiments, the antagonist and the second agent is administered simultaneously or concurrently. In some embodiments, the antagonist and the second agent is administered sequentially. In some embodiments, the antagonist and/or the second agent is administered parentally. In some embodiments, the antagonist is administered to diseased tissue directly.

In some embodiments, there is provided a method of treating a cancer (such as a solid tumor, a colon cancer, melanoma, or a T cell lymphoma) in an individual, comprising administering into the individual an effective amount of an antagonist that comprises a nucleic acid agent that inhibits the expression of PLA2G2D, wherein the individual has a) a high T cell infiltration (e.g., CD3 T cells, e.g., CD4 T cells, e.g., CD8 T cells, e.g., activated CD3 or CD4 or CD8 T cells) in the cancer tissue, and/or b) a high PLA2G2D expression in the cancer tissue.

In some embodiments, the methods described herein further comprise selecting an individual for treatment based upon high T cell infiltration (e.g., high CD3 T cells, high CD8 T cells, high CD4 T cells, activated T cells, activated CD8 T cells, or activated CD4 T cells) in the cancer tissue. High T cell infiltration can be determined by a) assessing the number of T cells (e.g., CD3 T cells. CD4 T cells, CD8 T cells, activated T cells, activated CD4 T cells, activated CD8 T cells) in tumor, and b) comparing the number to the number of corresponding T cells in a reference tissue. In some embodiments, the high T cell infiltration is present when the number of the T cells in the cancer is at least about 5%, 10%, 15%, 20%, 25%, 30%, 40%, 50%, 60%, 70%, 80%, 90%, or 95% more than the number of the corresponding T cells in a reference tissue. In some embodiments, the high T cell infiltration is present when the number of the T cells in the cancer is at least about 1-fold, 2-fold, 3-fold, 4-fold, 5-fold, 6-fold, 7-fold, 8-fold, 9-fold, or 10-fold more than the number of the corresponding T cells in a reference tissue. In some embodiments, the reference tissue is the corresponding tissue in a healthy individual. In some embodiments, the number of the corresponding T cells in a reference tissue is the average number of the corresponding T cells in the same tissue in a group of individuals (such as 10, 30, 50, 100 individuals) with same or similar cancer. In some embodiments, the reference tissue is the corresponding tissue in an individual who also has a cancer but has a less suppressed immune response in the cancer tissue as indicated by a biomarker. Examples of biomarker indicative of immunosuppressive tumor microenvironment (TME) includes: a) a high number, percentage and/or density of M2 macrophages (e.g., CD68+CD163+ cells) in the tissue; b) a high expression level of an immune checkpoint agent (e.g., PD-1 or PD-L1).

In some embodiments, the methods described above further comprise selecting an individual for treatment based upon a high expression level of PLA2G2D in the cancer tissue.

In some embodiments, the cancer tissue has a high expression level of PLA2G2D when the expression level of PLA2G2D (e.g., assessed by immunohistochemistry) is at least about 5%, 10%, 15%, 20%, 25%, 30%, 40%, 50%, 60%, 70%, 80%, 90%, or 95% higher than the expression level of PLA2G2D in a reference tissue. In some embodiments, the cancer tissue has a high expression level of PLA2G2D when the expression level of PLA2G2D (e.g., assessed by immunohistochemistry) is at least about 1-fold, 2-fold, 3-fold, 4-fold, 5-fold, 6-fold, 7-fold, 8-fold, 9-fold, 10-fold, 20-fold, 30-fold, 40-fold, or 50-fold higher than the expression level of PLA2G2D in a reference tissue. In some embodiments, the reference tissue is the corresponding tissue in a healthy individual. In some embodiments, the expression level of PLA2G2D in a reference tissue is the average expression level of PLA2G2D in the same tissue in a group of individuals (such as 10, 30, 50, 100 individuals) with same or similar cancer. In some embodiments, the reference tissue is the corresponding tissue in an individual who also has a cancer but has a less suppressed immune response in the cancer tissue as indicated by a biomarker (such as high M2 macrophages, or high expression of an immune checkpoint agent such as PD-1 or PD-L1).

In some embodiments, the methods described herein comprise selecting an individual for treatment, wherein the individual has a) a high T cell infiltration (e.g., CD3 T cells, e.g., CD4 T cells, e.g., CD8 T cells, e.g., activated CD3 or CD4 or CD8 T cells) in the cancer tissue, and/or b) a high PLA2G2D expression in the cancer tissue.

In some embodiments, there is provided a method of treating an infectious disease (such as a viral infectious disease) in an individual, comprising administering into the individual an effective amount of an antagonist that comprises a nucleic acid agent that inhibits the expression of PLA2G2D. In some embodiments, the nucleic acid agent comprises a siRNA, a miRNA, or an antisense RNA. In some embodiment, the PLA2G2D is a human PLA2G2D. In some embodiments, the infection site has an increased expression level of PLA2G2D as compared to a reference tissue (such as a corresponding tissue in a healthy individual). In some embodiments, the method further comprises administering a second agent. In some embodiments, the second agent comprises an immune therapy. In some embodiments, the antagonist and the second agent is administered simultaneously or concurrently. In some embodiments, the antagonist and the second agent is administered sequentially. In some embodiments, the antagonist and/or the second agent is administered parentally. In some embodiments, the antagonist is administered to diseased tissue directly.

In some embodiments, there is provided a method of treating a cancer (such as a solid tumor, a colon cancer, melanoma, or a T cell lymphoma) in an individual, comprising administering into the individual an effective amount of an antagonist decreasing enzymatic activity level of PLA2G2D. In some embodiments, the antagonist targeting PLA2G2D signaling pathway blocks a catalytic site on PLA2G2D. In some embodiment, the PLA2G2D is a human PLA2G2D. In some embodiments, the antagonist comprises an agent that specifically inhibits the catalytic His67-Asp68 Dyad of human PLA2G2D as set forth in SEQ ID NO: 1 or 5. In some embodiments, the antagonist targets the H67 catalytic site on a human PLA2G2D according to SEQ ID NO: 1 or 5. In some embodiments, the agent interferes with the binding of calcium to PLA2G2D. In some embodiments, the agent blocks the binding of calcium to residues at one or more of H47, G49, G51, and D68 according to SEQ ID NO: 1 or 5. In some embodiments, the antagonist comprises an agent that specifically decreases enzymatic activity of the catalytic His67-Asp68 Dyad of human PLA2G2D as set forth in SEQ ID NO: 1 or 5 by at least about 20%, 30%, 40%, 50%, 60%, 70%, 75%, 80%, 85%, 90%, or 95%. In some embodiments, the cancer tissue has an increased expression level of PLA2G2D as compared to a reference tissue (such as a corresponding tissue in a healthy individual). In some embodiments, the cancer is an advanced or malignant tumor. In some embodiments, the cancer is selected from the group consisting of lung cancer, breast cancer, liver cancer, gastric cancer, cervical cancer, endometrial cancer, thyroid cancer, colorectal cancer, head and neck cancer, pancreatic cancer, renal cancer, prostate cancer, urothelial cancer, testis cancer, ovarian cancer and melanoma. In some embodiments, the method further comprises administering a second agent. In some embodiments, the second agent is selected from the group consisting of a chemotherapeutic agent, an immunomodulator, an anti-angiogenesis agent, a growth inhibitory agent, and an antineoplastic agent. In some embodiments, the second agent is an immunomodulator. In some embodiments, the immunomodulator is an immune checkpoint inhibitor. In some embodiments, the immune checkpoint inhibitor specifically target PD-L1, PD-L2, CTLA4, PD-L2, PD-1, CD47, TIGIT, GITR, TIM3, LAG3, CD27, 4-1BB, or B7H4. In some embodiments, the second agent comprises a cell comprising a chimeric antigen receptor that specifically binds to a tumor antigen. In some embodiments, the antagonist and the second agent is administered simultaneously or concurrently. In some embodiments, the antagonist and the second agent is administered sequentially. In some embodiments, the antagonist and/or the second agent is administered parentally. In some embodiments, the antagonist is administered to diseased tissue directly.

In some embodiments, there is provided a method of treating an infectious disease (such as a viral infectious disease) in an individual, comprising administering into the individual an effective amount of an antagonist decreasing enzymatic activity level of PLA2G2D. In some embodiments, the PLA2G2D is a human PLA2G2D. In some embodiments, the antagonist targeting PLA2G2D signaling pathway blocks a catalytic site on PLA2G2D. In some embodiments, the antagonist comprises an agent that specifically inhibits the catalytic His67-Asp68 Dyad of human PLA2G2D as set forth in SEQ ID NO: 1 or 5. In some embodiments, the antagonist targets the H67 catalytic site on a human PLA2G2D according to SEQ ID NO: 1 or 5. In some embodiments, the agent interferes with the binding of calcium to PLA2G2D. In some embodiments, the agent blocks the binding of calcium to residues at one or more of H47, G49, G51, and D68 according to SEQ ID NO: 1 or 5. In some embodiments, the antagonist comprises an agent that specifically decreases enzymatic activity of the catalytic His67-Asp68 Dyad of human PLA2G2D as set forth in SEQ ID NO: 1 or 5 by at least about 20%, 30%, 40%, 50%, 60%, 70%, 75%, 80%, 85%, 90%, or 95%. In some embodiments, the infection site has an increased expression level of PLA2G2D as compared to a reference tissue (such as a corresponding tissue in a healthy individual). In some embodiments, the method further comprises administering a second agent. In some embodiments, the second agent comprises an immune therapy. In some embodiments, the antagonist and the second agent is administered simultaneously or concurrently. In some embodiments, the antagonist and the second agent is administered sequentially. In some embodiments, the antagonist and/or the second agent is administered parentally. In some embodiments, the antagonist is administered to diseased tissue directly.

The administration of the antagonists described herein can also be useful for promoting local immune response, promoting proliferation and/or activation of immune cells (such as T cells), and promoting a favorable tumor microenvironment. In some embodiments, there is provided a method of promoting local immune response in a cancer tissue in an individual having a cancer (such as a solid tumor), comprising administering any of the antagonists described herein. In some embodiments, there is provided a method of promoting local immune response in an infection site in an individual having an infection (such as a virus infection), comprising administering any of the antagonists described herein.

In some embodiments, there is provided a method of promoting proliferation and/or activation of T cells in a cancer tissue in an individual having a cancer (such as a solid tumor), comprising administering any of the antagonists described herein. In some embodiments, there is provided a method of promoting proliferation and/or activation of T cells in an infection site in an individual having an infection (such as a virus infection), comprising administering any of the antagonists described herein. In some embodiments, the T cells are CD4+ T cells. In some embodiments, the T cells are CD8+ T cells.

In some embodiments, there is provided a method of promoting a favorable tumor microenvironment in a cancer tissue in an individual having a cancer (such as a solid tumor), comprising administering any of the antagonists described herein. In some embodiments, there is provided a method of promoting a favorable microenvironment in an infection site in an individual having an infection (such as a virus infection), comprising administering any of the antagonists described herein. “Promoting favorable tumor microenvironment” generally refers to or comprises conversion of a tumor tissue that is resistant to a cancer therapy (such as an immunotherapy) to a tumor tissue that is less resistant to the cancer therapy.

### Antagonist Targeting PLA2G2D Signaling Pathway

The antagonist may be any of an antibody, a polypeptide, a peptide, a polynucleotide, a peptidomimetic, a natural product, a carbohydrate, an aptamer an avimer, an anticalin, a speigelmer, or a small molecule that targets (i.e., inhibits or downregulates) PLA2G2D signaling pathway. In some embodiments, the antagonist targets (i.e., inhibits or downregulates) PLA2G2D. Particular examples of what the agent may be are described below. In some embodiments, the antagonist is a fusion protein (such as a fusion protein that comprises a half-life extending domain (e.g., an Fc domain)).

### PLA2G2D

PLA2G2D (phospholipase A2 group IID, sPLA2-IID) is a secreted member of the phospholipase A2 family. Phospholipase A2 family members hydrolyze the sn-2 fatty acid ester bond of glycerophospholipids to produce lysophospholipids and free fatty acid. To date, 10 sPLA2isoforms (IB, IIA, IIC, IID, IIE, IIF, III, V, X, and XII) have been identified in mammals. These isoforms, except for group III isoforms, have a highly conserved catalytic site, a Ca 2+ binding loop, and a common molecular weight of 14-19 kDa. Of these sPLA2isoforms, sPLA2-IIA, sPLA2-IIC, sPLA2-IID, sPLA2-IIE, sPLA2-IIF, and sPLA 2-V have the same chromosomal locus (1p34-p36), which are often referred to as group II subfamily sPLA2. The biological feature of the group II subfamily sPLA2 is that almost all isoforms, except sPLA 2-IIC (a pseudogene in humans), are associated with inflammatory and immune processes.

Human PLA2G2D is a basic protein (pI˜8.7) with 14 cysteines at exactly conserved positions. Likely because of its cationic nature, PLA2G2D binds to heparin in vitro or heparin sulfate on the cell surface when overexpressed in cultured cells.

In some embodiments, the PLA2G2D comprises an amino acid sequence set forth in SEQ ID NO: 1 or 2. In some embodiments, the PLA2G2D comprises an amino acid sequence set forth in SEQ ID NO: 5 or 6.

### Antagonist Targeting PLA2G2D

In some embodiments, the antagonist decreases expression level of PLA2G2D. In some embodiments, the antagonist decreases enzymatic activity level of PLA2G2D. In some embodiments, the anti-PLA2G2D antibody does not completely inhibit or block the catalytic activity of PLA2G2D (such as blocking the catalytic activity no more than about 90%, 80%, 70%, 60%, 50%, 40%, 30%, 20% or 10% of the full catalytic activity). In some embodiments, the anti-PLA2G2D antibody does not inhibit or block the catalytic activity of PLA2G2D.

In some embodiments, the antagonist comprises an agent that inhibits PLA2G2D (such as an agent that blocks the binding of PLA2G2D to an immune cell or an agent that inhibits the activity of PLA2G2D) (such as a T cell, such as an activated T cell, such as an activated CD4+ T cell, such as an activated CD8+ T cell).

A. Agents Binding to PLA2G2D

In some embodiments, the antagonist is an agent that recognizes and binds specifically to PLA2G2D. In some embodiments, the agent comprises an anti-PLA2G2D antibody moiety (such as an anti-PLA2G2D antibody).

In some embodiments, the anti-PLA2G2D antibody moiety blocks or decreases the binding of PLA2G2D to an immune cell. In some embodiments, the anti-PLA2G2D antibody moiety decreases the binding of PLA2G2D to an immune cell by at least about 5%, 10%, 15%, 20%, 25%, 30%, 35%, 40%, 55%, 60%, 65%, 70%, 75%, 80%, 85%, 90%, or 95%. In some embodiments, the binding of the PLA2G2D to an immune cell is independent from binding through heparin sulfate on cell surface.

In some embodiments, the PLA2G2D recognized by the anti-PLA2G2D antibody is a human PLA2G2D. In some embodiments, the human PLA2G2D comprises or has the amino acid sequence of SEQ ID NO: 1 or a natural variant of human PLA2G2D. In some embodiments, the natural variant of human PLA2G2D is derived from a tumor tissue. In some embodiments, the natural variant of human PLA2G2D is derived from a virus infection site.

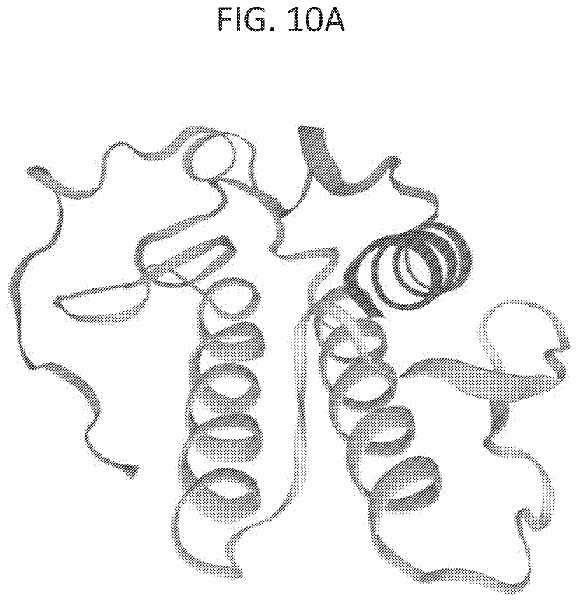

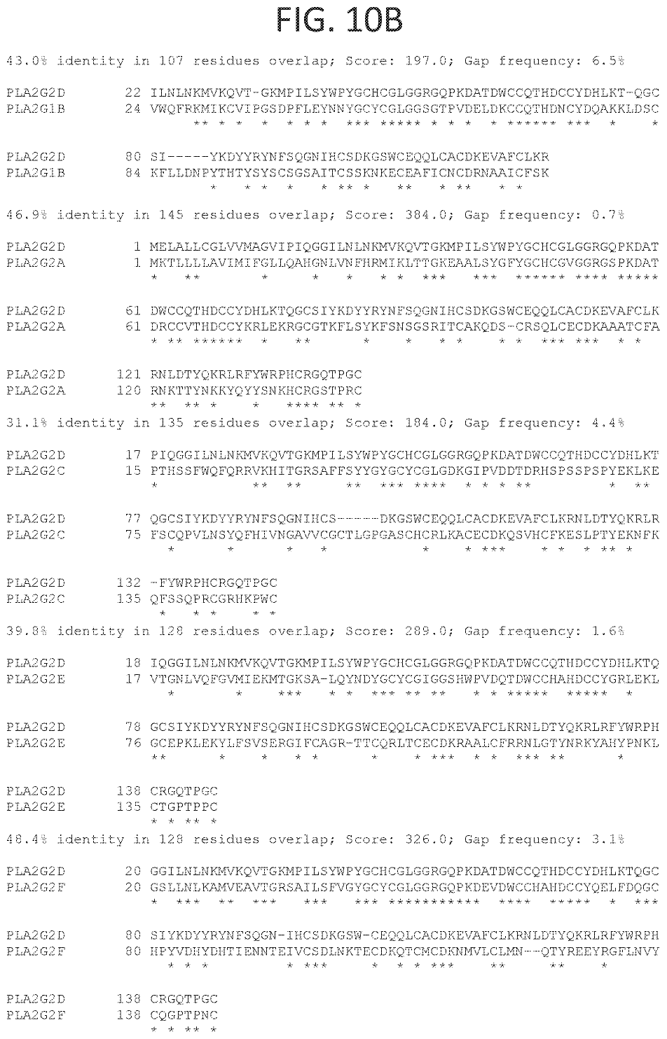

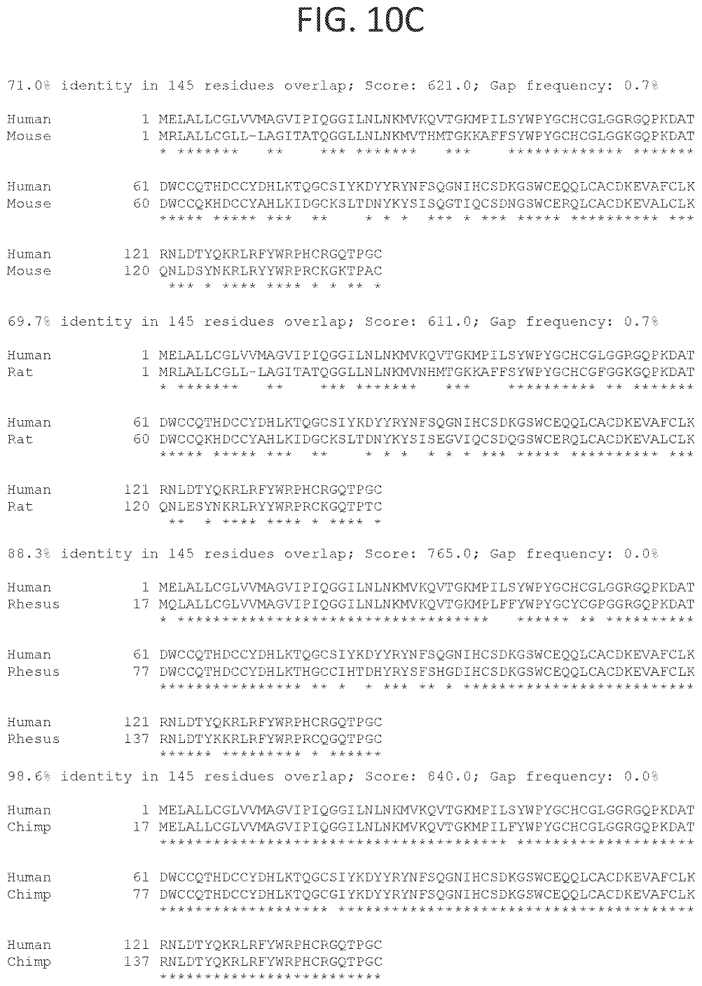

3D structure of the PLA2G2D predicted based upon Swiss-model is shown in FIG. 10A. In some embodiments, the anti-PLA2G2D antibody moiety binds to an epitope on PLA2G2D comprising any one or more of (such as one, two, three, four, or five of) Q65, H73, S80, H96, and R121 according to SEQ ID NO: 1. (Q65, H73, S80, H96 and R121 are sites that vary natural variants.) In some embodiments, the anti-PLA2G2D antibody moiety binds to an epitope on PLA2G2D comprising any one or more of (such as one, two, three, four, five or more of) amino acids from R121 to C145 according to SEQ ID NO: 1. In some embodiments, the anti-PLA2G2D antibody moiety binds to an epitope on PLA2G2D comprising any one or more of (such as one, two, three, four, five or more of) amino acids from V32 to A59 according to SEQ ID NO: 1. In some embodiments, the anti-PLA2G2D antibody moiety binds to an epitope on PLA2G2D comprising any one or more of (such as one, two, three, four, five or more of) amino acids from T60 to T76. In some embodiments, the anti-PLA2G2D antibody moiety binds to an epitope on PLA2G2D comprising any one or more of (such as one, two, three, four, five or more of) amino acids from Q77 to Y85 according to SEQ ID NO: 1. In some embodiments, the anti-PLA2G2D antibody moiety binds to an epitope on PLA2G2D comprising any one or more of (such as one, two, three, four, five or more of) amino acids from G21 to Q31 according to SEQ ID NO: 1. In some embodiments, the anti-PLA2G2D antibody moiety binds to an epitope on PLA2G2D comprising any one or more of (such as one, two, three, four, five or more of) amino acids from Y86 to W103 according to SEQ ID NO: 1. In some embodiments, the anti-PLA2G2D antibody moiety binds to an epitope on PLA2G2D comprising any one or more of (such as one, two, three, four, five or more of) amino acids from C104 to R121 according to SEQ ID NO: 1. In some embodiments, the epitope is a discontinuous epitope. In some embodiments, the epitope is a continuous epitope.

Sequence homology of human PLA2G2D to different PLA2 group 2 family members is analyzed and shown in FIG. 10B. Sequence homology of human PLA2G2D to PLA2G2D of different species is analyzed and shown in FIG. 10C. In some embodiments, the anti-PLA2G2D antibody moiety binds to an epitope on PLA2G2D comprising one or more residues at positions a) that are different from corresponding residues in other PLA2 group 2 family members and/or b) that are same as PLA2G2D in other species. In some embodiments, the anti-PLA2G2D antibody moiety binds to an epitope on PLA2G2D comprising any one or more of (such as one, two, three, four, five or more of) amino acids at the position of 22, 23, 25, 26, 27, or 31 according to SEQ ID NO: 1. In some embodiments, the anti-PLA2G2D antibody moiety binds to an epitope on PLA2G2D comprising any one or more of (such as one, two, three, four, five or more of) amino acids at the position of 36, 37, 38, 42, 43, 55, or 59 according to SEQ ID NO: 1. In some embodiments, the anti-PLA2G2D antibody moiety binds to an epitope on PLA2G2D comprising any one or more of (such as one, two, three, four, five or more of) amino acids at the position of 62, 65, 66, 72, 73, or 76, according to SEQ ID NO: 1. In some embodiments, the anti-PLA2G2D antibody moiety binds to an epitope on PLA2G2D comprising any one or more of (such as one, two, three, four, five or more of) amino acids at the position of 77, 80, 81, 83, 84, or 85 according to SEQ ID NO: 1. In some embodiments, the anti-PLA2G2D antibody moiety binds to an epitope on PLA2G2D comprising any one or more of (such as one, two, three, four, five or more of) amino acids at the position of 87, 89, 90, 92, 93, 94, 96, 98, 99, 100, 101, 102, or 103 according to SEQ ID NO: 1. In some embodiments, the anti-PLA2G2D antibody moiety binds to an epitope on PLA2G2D comprising any one or more of (such as one, two, three, four, five or more of) amino acids at the position of 105, 106, 107, 108, 110, 114, 115, 117, 119, or 120 according to SEQ ID NO: 1. In some embodiments, the anti-PLA2G2D antibody moiety binds to an epitope on PLA2G2D comprising any one or more of (such as one, two, three, four, five or more of) amino acids at the position of 123, 124, 127, 129, 130, 131, 132, 134, 135, 136, 137, 139, 141, 144, or 145 according to SEQ ID NO: 1. In some embodiments, the anti-PLA2G2D antibody moiety binds to an epitope on PLA2G2D comprising any one or more of (such as one, two, three, four, five or more of) amino acids at the position of 22, 26, 31, 36, 42, 43, 72, 73, 76, 77, 80, 81, 83, 85, 87, 89, 90, 92, 94, 96, 100, 101, 102, 103, 106, 110, 114, 115, 117, 120, 134, 135, 136, 141, or 144 according to SEQ ID NO: 1. In some embodiments, the epitope is a discontinuous epitope. In some embodiments, the epitope is a continuous epitope.

In some embodiments, the agent comprises an anti-PLA2G2D antibody. In some embodiments, the anti-PLA2G2D antibody is a polyclonal antibody. In some embodiments, the anti-PLA2G2D antibody is a monoclonal antibody.

In some embodiments, the anti-PLA2G2D antibody is an anti-human PLA2G2D antibody.

In some embodiments, the anti-PLA2G2D antibody is humanized or chimeric.

In some embodiments, the anti-PLA2G2D antibody is a full-length antibody or an immunoglobulin derivative. In some embodiments, the anti-PLA2G2D antibody is an antigen-binding fragment, for example an antigen-binding fragment selected from the group consisting of a single-chain Fv (scFv), a Fab, a Fab′, a F(ab′)2, an Fv fragment, a disulfide stabilized Fv fragment (dsFv), a (dsFv)₂, a V_{H}H, a Fv-Fc fusion, a scFv-Fc fusion, a scFv-Fv fusion, a diabody, a tribody, and a tetrabody. In some embodiments, the anti-PLA2G2D antibody is a scFv. In some embodiments, the anti-PLA2G2D antibody is a Fab or Fab′. In some embodiments, the anti-PLA2G2D antibody is chimeric, human, partially humanized, fully humanized, or semi-synthetic. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

In some embodiments, the anti-PLA2G2D antibody comprises an Fc fragment (such as any of the Fc fragments described herein). In some embodiments, the Fc fragment is selected from the group consisting of Fc fragments from IgG, IgA, IgD, IgE, IgM, and combinations and hybrids thereof. In some embodiments, the Fc fragment is derived from a human IgG. In some embodiments, the Fc fragment comprises the Fc region of human IgG1, IgG2, IgG3, IgG4, or a combination or hybrid IgG.

In some embodiments, the anti-PLA2G2D antibody does not completely inhibit or block the catalytic activity of PLA2G2D (such as blocking the catalytic activity no more than about 90%, 80%, 70%, 60%, 50%, 40%, 30%, 20% or 10% of the full catalytic activity). In some embodiments, the anti-PLA2G2D antibody does not inhibit or block the catalytic activity of PLA2G2D.

In some embodiments, the anti-PLA2G2D antibody blocks the binding of PLA2G2D to a T cell by at least about 5%, 10%, 15%, 20%, 25%, 30%, 35%, 40%, 45%, or 50%.

In some embodiments, the anti-PLA2G2D antibody is capable of restoring T cell activation to at least about 50%, 60%, 70%, 80%, 90%, 95%, or 100%. The activation of the T cell can be indicated, for example, by its cytokine secretion level. Exemplary cytokines include IL-2 and IFN-γ.

### Epitope Mapping

Determination of whether an antibody moiety binds within an epitope region can be carried out in ways known to the person skilled in the art. As one example of such mapping/characterization methods, an epitope region for an anti-PLA2G2D antibody may be determined by epitope “foot-printing” using chemical modification of the exposed amines/carboxyls in the PLA2G2D protein. One specific example of such a foot-printing technique is the use of HXMS (hydrogen-deuterium exchange detected by mass spectrometry) wherein a hydrogen/deuterium exchange of receptor and ligand protein amide protons, binding, and back exchange occurs, wherein the backbone amide groups participating in protein binding are protected from back exchange and therefore will remain deuterated. Relevant regions can be identified at this point by peptic proteolysis, fast microbore high-performance liquid chromatography separation, and/or electrospray ionization mass spectrometry. See, e.g., Ehring H. Analytical Biochemistry, Vol. 267 (2) pp. 252-259 (1999); Engen, J. R. and Smith, D. L. (2001) Anal. Chem. 73, 256A-265A, each of which is incorporated herein by reference in their entirety for all purposes. Another example of a suitable epitope identification technique is nuclear magnetic resonance epitope mapping (NMR), where typically the position of the signals in two-dimensional NMR spectra of the free antigen and the antigen complexed with the antigen binding peptide, such as an antibody, are compared. The antigen typically is selectively isotopically labeled with 15N so that only signals corresponding to the antigen and no signals from the antigen binding peptide are seen in the NMR-spectrum. Antigen signals originating from amino acids involved in the interaction with the antigen binding peptide typically will shift position in the spectrum of the complex compared to the spectrum of the free antigen, and the amino acids involved in the binding can be identified that way. See, e.g., Ernst Schering Res Found Workshop. 2004; (44): 149-67; Huang el al., Journal of Molecular Biology, Vol. 281 (1) pp. 61-67 (1998); and Saito and Patterson, Methods. 1996 June: 9 (3): 516-24, each of which is incorporated herein by reference in their entirety for all purposes.

Epitope mapping/characterization also can be performed using mass spectrometry methods. See, e.g., Downard, J Mass Spectrom. 2000 April; 35 (4): 493-503 and Kiselar and Downard, Anal Chem. 1999 May 1; 71 (9): 1792-1801, each of which is incorporated herein by reference in their entirety for all purposes. Protease digestion techniques also can be useful in the context of epitope mapping and identification. Antigenic determinant-relevant regions/sequences can be determined by protease digestion, e.g. by using trypsin in a ratio of about 1:50 to PLA2G2D or o/n digestion at and pH 7-8, followed by mass spectrometry (MS) analysis for peptide identification. The peptides protected from trypsin cleavage by the anti-PLA2G2D binder can subsequently be identified by comparison of samples individualed to trypsin digestion and samples incubated with antibody and then individualed to digestion by e.g. trypsin (thereby revealing a footprint for the binder). Other enzymes like chymotrypsin, pepsin, etc., also or alternatively can be used in similar epitope characterization methods. Moreover, enzymatic digestion can provide a quick method for analyzing whether a potential antigenic determinant sequence is within a region of the PLA2G2D polypeptide (such as a polypeptide set forth in SEQ ID NO: 1) that is not surface exposed and, accordingly, most likely not relevant in terms of immunogenicity/antigenicity.

Site-directed mutagenesis is another technique useful for elucidation of a binding epitope. For example, in “alanine-scanning”, each residue within a protein segment is re-placed with an alanine residue, and the consequences for binding affinity measured. If the mutation leads to a significant reduction in binding affinity, it is most likely involved in binding. Monoclonal antibodies specific for structural epitopes (i.e., antibodies which do not bind the unfolded protein) can be used to verify that the alanine-replacement does not influence over-all fold of the protein. See, e.g., Clackson and Wells, Science 1995; 267:383-386: and Wells, Proc Natl Acad Sci USA 1996; 93:1-6.

Electron microscopy can also be used for epitope “foot-printing”. For example, Wang et al., Nature 1992; 355:275-278 used coordinated application of cryoelectron micros-copy, three-dimensional image reconstruction, and X-ray crystallography to determine the physical footprint of a Fab-fragment on the capsid surface of native cowpea mosaic virus.

Other forms of “label-free” assay for epitope evaluation include surface plasmon resonance (SPR, BIACORE) and reflectometric interference spectroscopy (RifS). See, e.g., Fagerstam et al., Journal Of Molecular Recognition 1990:3:208-14: Nice et al., J. Chroma-togr. 1993; 646:159-168; Leipert et al., Angew. Chem. Int. Ed. 1998; 37:3308-3311: Kroger et al., Biosensors and Bioelectronics 2002; 17:937-944.

### Immunoconjugates

In some embodiments, the agents that bind to PLA2G2D described herein further comprises a second moiety. In some embodiments, the second moiety comprises a therapeutic agent. In some embodiments, the second moiety comprises a label. In some embodiments, the anti-PLA2G2D antibody moiety and the second moiety is linked via a linker (such as any of the linkers described in the “Linkers” section).

In some embodiments, the second agent is a cytotoxic agent. In some embodiments, the cytotoxic agent is a chemotherapeutic agent. In some embodiments, the cytotoxic agent is a growth inhibitory agent. In some embodiments, the cytotoxic agent is a toxin (e.g., protein toxins, enzymatically active toxins of bacterial, fungal, plant, or animal origin, or fragments thereof). In some embodiments, the cytotoxic agent is a radioactive isotype (i.e., a radioconjugage).

Immunoconjugates allow for the targeted delivery of a drug moiety to a tissues (such as a tumor), and, in some embodiments intracellular accumulation therein, where systemic administration of unconjugated drugs may result in unacceptable levels of toxicity to normal cells (Polakis P. (2005) Current Opinion in Pharmacology 5:382-387).

Antibody-drug conjugates (ADC) are targeted chemo therapeutic molecules which combine properties of both antibodies and cytotoxic drugs by targeting potent cytotoxic drugs to antigen-expressing tumor cells (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour: 14(3):154-169; Chari, R. V. (2008) ACC. Chen. Res. 41.98-107.

In the context of treating cancer, the ADC compounds of the application include those with anticancer activity. In some embodiments, the ADC compounds include an antibody conjugated, i.e. covalently attached, to the drug moiety. In some embodiments, the antibody is covalently attached to the drug moiety through a linker. In some embodiments, the second agent is connected to the anti-PLA2G2D antibody moiety via a linker (such as a linker described herein). In some embodiments, the linker is a cleavable. In some embodiments, the linker is non-cleavable.

The antibody-drug conjugates (ADC) of the application selectively deliver an effective dose of a drug to tumor tissue whereby greater selectivity, i.e. a lower efficacious dose, may be achieved while increasing the therapeutic index (“therapeutic window’). The drug moiety of the antibody-drug conjugates (ADC) may include any compound, moiety or group that has a cytotoxic or cytostatic effect. Drug moieties may impart their cytotoxic and cytostatic effects by mechanisms including but not limited to tubulin binding, DNA binding or intercalation, and inhibition of RNA polymerase, protein synthesis, and/or topoisomerase. Exemplary drug moieties include, but are not limited to, a maytansinoid, dolastatin, auristatin, calicheamicin, pyrrolobenzodiazepine (PBD), nemorubicin and its derivatives, PNU-159682, anthracy cline, duocarmycin, Vinca alkaloid, taxane, trichothecene, CC1065, camptothecin, elinafide, and stereoisomers, isosteres, analogs, and derivatives thereof that have cytotoxic activity.

Production of immunoconjugates described herein can be found in, for example, U.S. Pat. Nos. 9,562,099 and 7,541,034, which are hereby incorporated by references in their entirety.

### Fusion Proteins

In some embodiments, the agent binds to PLA2G2D comprises a fusion protein that comprises an anti-PLA2G2D antibody moiety and a second moiety.

In some embodiments, the second moiety comprises an Fc fragment (such as any of the Fc fragments described herein). In some embodiments, the half-life extending moiety is an albumin binding moiety (e.g., an albumin binding antibody moiety).

In some embodiments, the second moiety comprises a cytokine. In some embodiments, the cytokine is a proinflammatory cytokine (such as TNF-α, IL-1B, IL-6, or IL-10).

In some embodiments, the anti-PLA2G2D antibody moiety and the second moiety is linked via a linker (such as any of the linkers described in the “Linkers” section).

1. Fc Fragment

The term “Fc region,” “Fc domain” or “Fc” refers to a C-terminal non-antigen binding region of an immunoglobulin heavy chain that contains at least a portion of the constant region. The term includes native Fc regions and variant Fc regions. In some embodiments, a human IgG heavy chain Fc region extends from Cys226 to the carboxyl-terminus of the heavy chain. However, the C-terminal lysine (Lys447) of the Fc region may or may not be present, without affecting the structure or stability of the Fc region. Unless otherwise specified herein, numbering of amino acid residues in the IgG or Fc region is according to the EU numbering system for antibodies, also called the EU index, as described in Kabat et al., Sequences of Proteins of Immunological Interest, 5th Ed. Public Health Service, National Institutes of Health, Bethesda, Md., 1991.

In some embodiments, the Fc fragment is selected from the group consisting of Fc fragments from IgG, IgA, IgD, IgE, IgM, and combinations and hybrids thereof. In some embodiments, the Fc fragment is selected from the group consisting of Fc fragments from IgG1, IgG2, IgG3, IgG4, and combinations and hybrids thereof.

In some embodiments, the Fc fragment has a reduced effector function as compared to corresponding wildtype Fc fragment (such as at least about 30%, 40%, 50%, 60%, 70%, 80%, 85%, 90%, or 95% reduced effector function as measured by the level of antibody-dependent cellular cytotoxicity (ADCC)).

In some embodiments, the Fc fragment is an IgG1 Fc fragment. In some embodiments, the IgG1 Fc fragment comprises a L234A mutation and/or a L235A mutation. In some embodiments, the Fc fragment is an IgG2 or IgG4 Fc fragment. In some embodiments, the Fc fragment is an IgG4 Fc fragment comprising a S228P, F234A, and/or a L235A mutation. In some embodiments, the Fc fragment comprises a N297A mutation. In some embodiments, the Fc fragment comprises a N297G mutation.

2. Linkers

In some embodiments, the anti-PLA2G2D immunoconjugates or fusion proteins described herein comprise an anti-PLA2G2D antibody described herein fused to the second moiety via a linker.

The length, the degree of flexibility and/or other properties of the linker used in the anti-PLA2G2D immunoconjugates or fusion proteins may have some influence on properties, including but not limited to the affinity, specificity or avidity of the anti-PLA2G2D, and/or affinity, specificity or avidity for one or more particular antigens or epitopes present on PLA2G2D. For example, longer linkers may be selected to ensure that two adjacent antibody moieties do not sterically interfere with one another. In some embodiments, a linker (such as peptide linker) comprises flexible residues (such as glycine and serine) so that the adjacent antibody moieties are free to move relative to each other. For example, a glycine-serine doublet can be a suitable peptide linker. In some embodiments, the linker is a non-peptide linker. In some embodiments, the linker is a peptide linker. In some embodiments, the linker is a non-cleavable linker. In some embodiments, the linker is a cleavable linker.

Other linker considerations include the effect on physical or pharmacokinetic properties of the resulting an anti-PLA2G2D immunoconjugate or fusion protein, such as solubility, lipophilicity, hydrophilicity, hydrophobicity, stability (more or less stable as well as planned degradation), rigidity, flexibility, immunogenicity, modulation of antibody binding, the ability to be incorporated into a micelle or liposome, and the like.

### Peptide Linkers

Any one or all of the linkers described herein can be peptide linkers. The peptide linker may have a naturally occurring sequence, or a non-naturally occurring sequence. For example, a sequence derived from the hinge region of heavy chain only antibodies may be used as the linker. See, for example, WO1996/34103, incorporated by reference in its entirety for all purposes. In some embodiments, the peptide linker comprises the amino acid sequence of CPPCP, a sequence found in the native IgG1 hinge region.

The peptide linker can be of any suitable length. In some embodiments, the length of the peptide linker is any of about 1 aa to about 10 aa, about 1 aa to about 20 aa, about 1 aa to about 30 aa, about 5 aa to about 15 aa, about 10 as to about 25 aa, about 5 as to about 30 aa, about 10 as to about 30 aa, about 30 as to about 50 aa, about 50 as to about 100 aa, or about 1 as to about 100 aa.

An essential technical feature of such peptide linker is that said peptide linker does not comprise any polymerization activity. The characteristics of a peptide linker, which comprise the absence of the promotion of secondary structures, are known in the art and described, e.g., in Dall' Acqua et al. (Biochem. (1998) 37, 9266-9273), Cheadle et al. (Mol Immunol (1992) 29, 21-30) and Raag and Whitlow (FASEB (1995) 9(1), 73-80, each incorporated by reference in their entirety for all purposes). A particularly preferred amino acid in context of the “peptide linker” is Gly. Furthermore, peptide linkers that also do not promote any secondary structures are preferred. The linkage of the molecules to each other can be provided by, e.g., genetic engineering. Methods for preparing fused and operatively linked antibody constructs and expressing them in mammalian cells or bacteria are well-known in the art (e.g. WO 99/54440, Ausubel, Current Protocols in Molecular Biology, Green Publishing Associates and Wiley Interscience, N.Y. 1989 and 1994 or Sambrook et al., Molecular Cloning: A Laboratory Manual, Cold Spring Harbor Laboratory Press, Cold Spring Harbor, N.Y., 2001, each incorporated by reference in their entirety for all purposes).

In some embodiments, the peptide linker is a stable linker, which is not cleavable by protease, such as by Matrix metalloproteinases (MMPs).

In some embodiments, the peptide linker tends not to adopt a rigid three-dimensional structure, but rather provide flexibility to a polypeptide (e.g., first and/or second components), such as providing flexibility between the anti-PLA2G2D and the second moiety. In some embodiments, the peptide linker is a flexible linker. Exemplary flexible linkers include glycine polymers (G)ₙ (SEQ ID NO: 13), glycine-serine polymers (including, for example, (GS)ₙ (SEQ ID NO: 14), (GSGGS)ₙ (SEQ ID NO: 15), (GGGGS)ₙ (SEQ ID NO: 16), and (GGGS)ₙ (SEQ ID NO: 17), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11 173-142 (1992)). The ordinarily skilled artisan will recognize that design of an anti-PLA2G2D can include linkers that are all or partially flexible, such that the linker can include a flexible linker portion as well as one or more portions that confer less flexible structure to provide a desired immunoconjugate or fusion protein structure.

Furthermore, exemplary linkers also include the amino acid sequence of such as (GGGGS)ₙ(SEQ ID NO: 16), wherein n is an integer between 1 and 8, e.g. (GGGGS)₃ (SEQ ID NO: 18; hereinafter referred to as “(G4S)3” or “GS3”), or (GGGGS)₆ (SEQ ID NO: 19; hereinafter referred to as “(G4S)6” or “GS6”). In some embodiments, the peptide linker comprises the amino acid sequence of (GSTSGSGKPGSGEGS)ₙ(SEQ ID NO: 20), wherein n is an integer between 1 and 3.

Natural linkers adopt various conformations in secondary structure, such as helical, β-strand, coil/bend and turns, to exert their functions. Linkers in an α-helix structure might serve as rigid spacers to effectively separate protein domains, thus reducing their unfavorable interactions. Non-helical linkers with Pro-rich sequence could increase the linker rigidity and function in reducing inter-domain interference. In some embodiments, the anti-PLA2G2D antibody moiety and the second moiety is linked together by an α-helical linker with an amino acid sequence of A(EAAAK)₄A (SEQ ID NO: 21).

### Non-Peptide Linkers

Any one or all of the linkers described herein can be accomplished by any chemical reaction that will bind the two molecules so long as the components or fragments retain their respective activities, e.g. binding to target PLA2G2D, function of the second moiety (such as binding to a FcR or cytokine receptor). This linkage can include many chemical mechanisms, for instance covalent binding, affinity binding, intercalation, coordinate binding and complexation. In some embodiments, the binding is covalent binding. Covalent binding can be achieved either by direct condensation of existing side chains or by the incorporation of external bridging molecules. Many bivalent or polyvalent linking agents are useful in coupling protein molecules, such as a second moiety to the anti-PLA2G2D antibody of the present invention. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents (see Killen and Lindstrom, Jour. Immun. 133:1335-2549 (1984); Jansen el al., Immunological Reviews 62:185-216 (1982); and Vitetta et al., Science 238:1098 (1987), each incorporated by reference in their entirety for all purposes).

Linkers that can be applied in the present application are described in the literature (see, for example, Ramakrishnan, S. et al., Cancer Res. 44:201-208 (1984) describing use of MBS (M-maleimidobenzoyl-N-hydroxysuccinimide ester), incorporated by reference in its entirety for all purposes). In some embodiments, non-peptide linkers used herein include: (i) EDC (1-ethyl-3-(3-dimethylamino-propyl) carbodiimide hydrochloride: (ii) SMPT (4-succinimidyloxycarbonyl-alpha-methyl-alpha-(2-pyridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G): (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus leading to agents binding to PLA2G2D (such as anti-PLA2G2D immunoconjugates or fusion proteins) with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form fusion protein with increased stability. Disulfide linkages, are in general, less stable than other linkages because the disulfide linkage is cleaved in vitro, resulting in less fusion protein available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

B. Inhibitory PLA2G2D Polypeptides or Variants Thereof

The methods described herein in some embodiments involve use of inhibitory PLA2G2D polypeptides that block the binding between PLA2G2D (e.g., a wildtype PLA2G2D) and immune cells completely or partially (such as blocks the binding between the PLA2G2D and an immune cell by at least about 5%, 10%, 15%, 20%, 25%, 30%, 35%, 40%, 45%, 50%, 60%, 70%, 80%, 90%, or 95%). The present application in one aspect provides novel and non-naturally occurring polypeptide comprising an inhibitory PLA2G2D polypeptide that blocks the binding of PLA2G2D to an immune cell. In some embodiments, the inhibitory PLA2G2D polypeptide is a soluble polypeptide.

In some embodiments, the inhibitory PLA2G2D polypeptide is membrane bound. In some embodiments, the membrane bound inhibitory PLA2G2D polypeptide binds to the immune cell but does not trigger PLA2G2D signaling pathway in the immune cell. In some embodiments, the membrane bound inhibitory PLA2G2D polypeptide binds to the immune cell and attenuates PLA2G2D signaling pathway in the immune cell. In some embodiments, the membrane bound inhibitory PLA2G2D polypeptide is introduced by a gene editing system or an mRNA delivery vehicle.

In some embodiments, the inhibitory PLA2G2D polypeptide comprises a naturally occurring PLA2G2D polypeptide. In some embodiments, the naturally occurring PLA2G2D polypeptide is from a human who has an autoimmune or inflammatory disease (such as chronic obstructive pulmonary disease (COPD)). In some embodiments, the inhibitory PLA2G2D polypeptides has a mutation at a position corresponding to a polymorphism described in Takabatake et al. (Am J Respir Crit Care Med. 2005 Nov. 1; 172(9):1097-104) or Igarashi et al. (Respiration. 2009; 78(3):312-21).

In some embodiments, the inhibitory PLA2G2D polypeptide comprises a mutation at the position corresponding to histidine at position 67 (H67) according to SEQ ID NO: 1 or 5.

In some embodiments, the inhibitory PLA2G2D polypeptide comprises an amino acid sequence of SEQ ID NO: 3, 4, 7, or 8 or a variant thereof. In some embodiments, the variant has at least about 80% (such as about 85%, 90%, 91%, 92%, 93%, 94%, 95%, 96%, 97%, 98%, or 99%) sequence identity to the amino acid sequence of SEQ ID NO: 3, 4, 7, or 8.

In some embodiments, the inhibitory PLA2G2D polypeptide comprises a mutation at a position corresponding to G80 according to SEQ ID NO: 5. In some embodiments, the inhibitory PLA2G2D polypeptide comprises a comprises an amino acid sequence of SEQ ID NO: 9 or 10 or a variant thereof. In some embodiments, the variant has at least about 80% (such as about 85%, 90%, 91%, 92%, 93%, 94%, 95%, 96%, 97%, 98%, or 99%) sequence identity to the amino acid sequence of SEQ ID NO: 9 or 10.

In some embodiments, the inhibitory PLA2G2D polypeptide comprises a) a mutation at the position corresponding to histidine at position 67 (H67) and b) a mutation at a position corresponding to G80 according to SEQ ID NO: 5. In some embodiments, the inhibitory PLA2G2D polypeptide comprises a comprises an amino acid sequence of SEQ ID NO: 11 or 12 or a variant thereof. In some embodiments, the variant has at least about 80% (such as about 85%, 90%, 91%, 92%, 93%, 94%, 95%, 96%, 97%, 98%, or 99%) sequence identity to the amino acid sequence of SEQ ID NO: 11 or 12.

In some embodiments, the inhibitory PLA2G2D polypeptide further comprises at least one or more (such as about at least 10, 15, 20, 25, 30, 35, 45, 50, or all) of residues at the position of 22, 23, 25, 26, 27, 31, 36, 37, 38, 42, 43, 55, 59, 62, 65, 66, 72, 73, 76, 77, 80, 81, 83, 84, 85, 87, 89, 90, 92, 93, 94, 96, 98, 99, 100, 101, 102, 103, 105, 106, 107, 108, 110, 114, 115, 117, 119, 120, 123, 124, 127, 129, 130, 131, 132, 134, 135, 136, 137, 139, 141, 144, and 145 wherein the amino acid numbering is based on SEQ ID NO:1.

In some embodiments, the inhibitory PLA2G2D polypeptide further comprises at least one or more (such as about at least 10, 15, 20, 25, 30, or all) of residues at the position of 22, 26, 31, 36, 42, 43, 72, 73, 76, 77, 80, 81, 83, 85, 87, 89, 90, 92, 94, 96, 100, 101, 102, 103, 106, 110, 114, 115, 117, 120, 134, 135, 136, 141, or 144 wherein the amino acid numbering is based on SEQ ID NO:1.

In some embodiments, the variant described herein is a natural variant. In some embodiments, the variant does not comprise a non-conservative substitution. In some embodiments, the variant only comprises one or more conservative substitution. In some embodiments, the one or more conservative substitutions comprise or consist of the substitutions shown in Table I below under the heading of “Preferred substitutions.”

TABLE 1
Amino acid substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

In some embodiments, the inhibitory PLA2G2D polypeptide binds to the immune cell with a greater affinity than for a wildtype PLA2G2D. In some embodiments, the inhibitory PLA2G2D polypeptide binds to the immune cell with a K_{D} of at most half, one-fifth, one-tenth, one-twentieth, one-fiftieth, one-hundredth, one-thousandth of that of the binding between the wildtype PLA2G2D and the immune cell.

In some embodiments, the inhibitory PLA2G2D polypeptide further comprises a stabilizing domain. The stabilizing domain can be any domain that stabilizes the inhibitory PLA2G2D polypeptide (for example, extending half-life of the inhibitory PLA2G2D polypeptide in vivo). In some embodiments, the stabilizing domain comprises an Fc fragment. Exemplary Fc fragment include those described under “Fc fragment” section.

In some embodiments, the inhibitory PLA2G2D polypeptide is about 50 to about 1000 amino acids in length, such as about 50-800, 50-500, 50-400, 50-300 or 50-200 amino acids in length. In some embodiments, the inhibitory polypeptide is about 50 to about 100 amino acids, about 100 to about 150 amino acids, or about 150 amino acids to about 200 amino acids in length.

C. Nucleic Acid Agents Targeting PLA2G2D

In some embodiments, the antagonist targeting PLA2G2D comprises a nucleic acid agent (such as a siRNA, a shRNA, a miRNA, or an antisense RNA) that targets PLA2G2D (such as a human PLA2G2D).

In some embodiments, the antagonist comprises a siRNA or RNAi. In some embodiments, the antagonist comprises an antisense RNA. In some embodiments, the antagonist comprises a short hairpin ribonucleic acid (shRNA). In some embodiments, the antagonist comprises a microRNA (miRNA).

One skilled in the art may select an interfering RNA (RNAi) or siRNA specific targeting PLA2G2D. The nucleic acid selected sometimes is the RNAi or siRNA or a nucleic acid that encodes such products. The term “RNAi” as used herein refers to double-stranded RNA (dsRNA) which mediates degradation of specific mRNAs, and can also be used to lower or eliminate gene expression. The term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule directed against a gene. For example, a siRNA is capable of inhibiting or down regulating gene expression or viral replication, for example by mediating RNA interference “RNAi” or gene silencing in a sequence-specific manner; see for example Zamore et al., 2000, Cell, 101, 25-33; Bass, 2001, Nature, 411, 428-429; Elbashir et al., 2001, Nature, 411, 494-498; and Kreutzer et al., International PCT Publication No. WO 00/44895: Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002. RNA, 8, 842-850; Reinhart et al., 2002, Gene & Dev., 16, 1616-1626; and Reinhart & Bartel, 2002, Science, 297, 1831). There is no particular limitation in the length of siRNA as long as it does not show toxicity. Examples of modified RNAi and siRNA include STEALTH™ forms (Invitrogen Corp., Carlsbad, Calif.), forms described in U.S. Patent Publication No. 2004/0014956 (application Ser. No. 10/357,529) and U.S. patent application Ser. No. 111/049,636, filed Feb. 2, 2005), and other forms described hereafter.

A siRNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siRNA can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double stranded structure, for example wherein the double stranded region is about 19 base pairs); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the siRNA is assembled from a single oligonucleotide, where the self-complementary sense and antisense regions of the siRNA are linked by means of a nucleic acid based or non-nucleic acid-based linker(s). The siRNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The siRNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNAi. The siRNA can also comprise a single stranded polynucleotide having nucleotide sequence complementary to nucleotide sequence in a target nucleic acid molecule or a portion thereof (for example, where such siRNA molecule does not require the presence within the siRNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate. In certain embodiments, the siRNA molecule of the invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic interactions, and/or stacking interactions. In certain embodiments, the siRNA molecules of the invention comprise nucleotide sequence that is complementary to nucleotide sequence of a target gene. In another embodiment, the siRNA molecule of the invention interacts with nucleotide sequence of a target gene in a manner that causes inhibition of expression of the target gene.

The double-stranded RNA portions of siRNAs in which two RNA strands pair are not limited to the completely paired forms, and may contain non-pairing portions due to mismatch (the corresponding nucleotides are not complementary), bulge (lacking in the corresponding complementary nucleotide on one strand), and the like. Non-pairing portions can be contained to the extent that they do not interfere with siRNA formation. The “bulge” used herein often comprises 1 to 2 non-pairing nucleotides, and the double-stranded RNA region of siRNAs in which two RNA strands pair up sometimes contains I to 7, and at times 1 to 5 bulges. In addition, the “mismatch” used herein is contained in the double-stranded RNA region of siRNAs in which two RNA strands pair up, sometimes I to 7, and at times 1 to 5, in number. In an often utilized mismatch, one of the nucleotides is guanine, and the other is uracil. Such a mismatch is due to a mutation from C to T. G to A, or mixtures thereof in DNA coding for sense RNA, but not particularly limited to them. Furthermore, in the present invention, the double-stranded RNA region of siRNAs in which two RNA strands pair up may contain both bulge and mismatched, which sum up to, sometimes 1 to 7, and at times 1 to 5, in number. The terminal structure of siRNA may be either blunt or cohesive (overhanging) as long as siRNA enables to silence the target gene expression due to its RNAi effect.

As used herein, siRNA molecules need not be limited to those molecules containing only RNA, but further encompasses chemically-modified nucleotides and non-nucleotides. In addition, as used herein, the term RNAi is meant to be equivalent to other terms used to describe sequence specific RNA interference, such as post transcriptional gene silencing, translational inhibition, or epigenetics. For example, siRNA molecules of the invention can be used to epigenetically silence genes at both the post-transcriptional level and the pre-transcriptional level. In a non-limiting example, epigenetic regulation of gene expression by siRNA molecules of the invention can result from siRNA mediated modification of chromatin structure to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002. Science, 297, 1833-1837; Jenuwein, 2002, Science, 297, 2215-2218: and Hall et al., 2002, Science, 297, 2232-2237).

RNAi may be designed by those methods known to those of ordinary skill in the art. In one example, siRNA may be designed by classifying RNAi sequences, for example 1000 sequences, based on functionality, with a functional group being classified as having greater than 85% knockdown activity and a non-functional group with less than 85% knockdown activity. The distribution of base composition was calculated for entire the entire RNAi target sequence for both the functional group and the non-functional group. The ratio of base distribution of functional and non-functional group may then be used to build a score matrix for each position of RNAi sequence. For a given target sequence, the base for each position is scored, and then the log ratio of the multiplication of all the positions is taken as a final score. Using this score system, a very strong correlation may be found of the functional knockdown activity and the log ratio score. Once the target sequence is selected, it may be filtered through both fast NCBI blast and slow Smith Waterman algorithm search against the Unigene database to identify the gene-specific RNAi or siRNA. Sequences with at least one mismatch in the last 12 bases may be selected.

An antisense nucleic acid can be designed, prepared and/or utilized by the artisan to inhibit a nucleic acid encoding PLA2G2D. An “antisense” nucleic acid refers to a nucleotide sequence complementary to a “sense” nucleic acid encoding PLA2G2D or fragment (e.g., complementary to the coding strand of a double-stranded cDNA molecule or complementary to an mRNA sequence). The antisense nucleic acid can be complementary to an entire coding strand, or to a portion thereof or a substantially identical sequence thereof. In another embodiment, the antisense nucleic acid molecule is antisense to a “noncoding region” of the coding strand of a nucleotide sequence.

An antisense nucleic acid can be complementary to the entire coding region of an mRNA encoded by a PLA2G2D nucleotide sequence, and often the antisense nucleic acid is an oligonucleotide antisense to only a portion of a coding or noncoding region of the mRNA. For example, the antisense oligonucleotide can be complementary to the region surrounding the translation start site of the mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length. An antisense nucleic acid can be constructed using chemical synthesis or enzyme ligation reactions using standard procedures. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used). Antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

When utilized in subjects, antisense nucleic acids typically are administered to a subject (e.g., by direct injection at a tissue site) or generated in situ such that they hybridize with or bind to cellular mRNA and/or genomic DNA encoding a polypeptide and thereby inhibit expression of the polypeptide, for example, by inhibiting transcription and/or translation. Alternatively, antisense nucleic acid molecules can be modified to target selected cells and then are administered systemically. For systemic administration, antisense molecules can be modified such that they specifically bind to receptors or antigens expressed on a selected cell surface, for example, by linking antisense nucleic acid molecules to peptides or antibodies which bind to cell surface receptors or antigens. Antisense nucleic acid molecules can also be delivered to cells using the vectors described herein. Sufficient intracellular concentrations of antisense molecules are achieved by incorporating a strong promoter, such as a pol II or pol III promoter, in the vector construct. Antisense nucleic acid molecules sometimes are alpha-anomeric nucleic acid molecules. An alpha-anomeric nucleic acid molecule forms specific double-stranded hybrids with complementary RNA in which, contrary to the usual beta-units, the strands run parallel to each other (Gaultier et al., Nucleic Acids. Res. 15: 6625-6641 (1987)). Antisense nucleic acid molecules also can comprise a 2′-o-methylribonucleotide (Inoue et al., Nucleic Acids Res. 15: 6131-6148 (1987)) or a chimeric RNA-DNA analogue (Inoue et al., FEBS Lett. 215: 327-330 (1987)). Antisense nucleic acids sometimes are composed of DNA or PNA or any other nucleic acid derivatives described previously.

An antisense nucleic acid is a ribozyme in some embodiments. A ribozyme having specificity for an Aid nucleotide sequence can include one or more sequences complementary to such a nucleotide sequence, and a sequence having a known catalytic region responsible for mRNA cleavage (e.g., U.S. Pat. No. 5,093,246 or Haselhoff and Gerlach, Nature 334: 585-591 (1988)). For example, a derivative of a Tetrahymena L-19 IVS RNA is sometimes utilized in which the nucleotide sequence of the active site is complementary to the nucleotide sequence to be cleaved in an mRNA (e.g., Cech et al. U.S. Pat. No. 4,987,071: and Cech et al. U.S. Pat. No. 5,116,742). PLA2G2D mRNA sequences also may be utilized to select a catalytic RNA having a specific ribonuclease activity from a pool of RNA molecules (e.g., Bartel & Szostak, Science 261: 1411-1418 (1993)).

In some embodiments, the nucleic acid agents targeting PLA2G2D are nucleic acids that can form triple helix structures with an Aid nucleic acid. PLA2G2D expression can be inhibited by targeting nucleotide sequences complementary to the regulatory region of a nucleotide sequence referenced herein or a substantially identical sequence (e.g., promoter and/or enhancers) to form triple helical structures that prevent transcription of a gene in target cells (see e.g., Helene, Anticancer Drug Des. 6(6): 569-84 (1991): Helene et al., Ann. N.Y. Acad. Sci. 660: 27-36 (1992): and Maher, Bioassays 14(12): 807-15 (1992). Triple helix formation can be enhanced by generating a “switchback” nucleic acid molecule. Switchback molecules are synthesized in an alternating 5′-3′,3′-5′ manner, such that they base pair with first one strand of a duplex and then the other, eliminating the necessity for a sizeable stretch of purines or pyrimidines being present on one strand of a duplex.

D. Genome-Editing Systems that Target PLA2G2D

In some embodiments, the antagonist targeting PLA2G2D comprises a genome-editing system that targets PLA2G2D. In some embodiments, the genome-editing system comprises a DNA nuclease such as an engineered (e.g., programmable or targetable) DNA nuclease to induce genome editing of a target DNA sequence of PLA2G2D. Any suitable DNA nuclease can be used including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof. In some embodiments, the genome editing comprises modifying PLA2G2D so that the modified PLA2G2D no longer suppresses an immune cell (such as a T cell, such as an activated T cell, such as an activated CD4+ T cell, such as an activated CD8+ T cell) or suppresses the immune cell to a less extent than wildtype PLA2G2D. In some embodiments, the genome editing comprises modifying PLA2G2D so that the modified PLA2G2D no longer binds to an immune cell (such as a T cell, such as an activated T cell, such as an activated CD4+ T cell, such as an activated CD8+ T cell) or binds to the immune cell to a less extent than wildtype PLA2G2D. In some embodiments, the modification comprises inserting a transgene comprising a variant of PLA2G2D (such as any of the variants of PLA2G2D described herein). In some embodiments, the variant PLA2G2D has a mutation at H67 based upon SEQ ID NO: 1. In some embodiments, the variant PLA2G2D has a H67A mutation based upon SEQ ID NO: 1.

E. Agents Inhibiting PLA2G2D Enzymatic Activity

In some embodiments, the antagonist comprises an agent that inhibits PLA2G2D enzymatic activity (i.e., hydrolyzing fatty acids). In some embodiments, the antagonist comprises an agent that specifically inhibits enzymatic activity of the catalytic His67-Asp68 Dyad of human PLA2G2D as set forth in SEQ ID NO: 1 or 5. In some embodiments, the antagonist comprises an agent that specifically decreases enzymatic activity of the catalytic His67-Asp68 Dyad of human PLA2G2D as set forth in SEQ ID NO: 1 or 5 by at least about 20%, 30%, 40%, 50%, 60%, 70%, 75%, 80%, 85%, 90%, or 95%.

In some embodiments, the agent interferes with the binding of calcium to PLA2G2D. In some embodiments, the agent blocks the binding of calcium to residues at one or more of H47, G49, G51, and D68 according to SEQ ID NO: 1 or 5.

### Disease or Condition

The methods described herein are applicable to diseases and conditions for which there are suppressed immune responses in the body that at least partly contribute to the less effective treating of the disease. Exemplary diseases include cancer or infectious disease (such as viral infectious disease).

### Cancer

In some embodiments, the disease or condition described herein is a cancer. Cancers that may be treated using any of the methods described herein include any types of cancers. Types of cancers to be treated with the agent as described in this application include, but are not limited to, carcinoma, blastoma, sarcoma, benign and malignant tumors, and malignancies e.g., sarcomas, carcinomas, and melanomas. Adult tumors/cancers and pediatric tumors/cancers are also included.

In various embodiments, the cancer is early stage cancer, non-metastatic cancer, primary cancer, advanced cancer, locally advanced cancer, metastatic cancer, cancer in remission, recurrent cancer, cancer in an adjuvant setting, cancer in a neoadjuvant setting, or cancer substantially refractory to a therapy.

In some embodiments, the cancer is a solid tumor.

In some embodiments, the cancer is a liquid tumor.

In some embodiments, the cancer tissue has a high expression level of PLA2G2D when the expression level of PLA2G2D (e.g., assessed by immunohistochemistry) is at least about 5%, 10%, 15%, 20%, 25%, 30%, 40%, 50%, 60%, 70%, 80%, 90%, or 95% higher than the expression level of PLA2G2D in a reference tissue. In some embodiments, the cancer tissue has a high expression level of PLA2G2D when the expression level of PLA2G2D (e.g., assessed by immunohistochemistry) is at least about 1-fold, 2-fold, 3-fold, 4-fold, 5-fold, 6-fold, 7-fold, 8-fold, 9-fold, 10-fold, 20-fold, 30-fold, 40-fold, or 50-fold higher than the expression level of PLA2G2D in a reference tissue. In some embodiments, the reference tissue is the corresponding tissue in a healthy individual. In some embodiments, the expression level of PLA2G2D in a reference tissue is the average expression level of PLA2G2D in the same tissue in a group of individuals (such as 10, 30, 50, 100 individuals) with same or similar cancer. In some embodiments, the reference tissue is the corresponding tissue in an individual who also has a cancer but has a less suppressed immune response in the cancer tissue as indicated by a biomarker (such as high M2 macrophages, or high expression of an immune checkpoint agent such as PD-1 or PD-L1).

In some embodiments, the cancer tissue has a high T cell infiltration (e.g., high CD3 T cells, high CD8 T cells, high CD4 T cells, activated T cells, activated CD8 T cells, or activated CD4 T cells) in the cancer tissue. In some embodiments, the cancer tissue has a high T cell infiltration when the number of the T cells in the cancer is at least about 5%, 10%, 15%, 20%, 25%, 30%, 40%, 50%, 60%, 70%, 80%, 90%, or 95% more than the number of the corresponding T cells in a reference tissue. In some embodiments, the high T cell infiltration is present when the number of the T cells in the cancer is at least about 1-fold, 2-fold, 3-fold, 4-fold, 5-fold, 6-fold, 7-fold, 8-fold, 9-fold, or 10-fold more than the number of the corresponding T cells in a reference tissue. In some embodiments, the reference tissue is the corresponding tissue in a healthy individual. In some embodiments, the number of the corresponding T cells in a reference tissue is the average number of the corresponding T cells in the same tissue in a group of individuals (such as 10, 30, 50, 100 individuals) with same or similar cancer. In some embodiments, the reference tissue is the corresponding tissue in an individual who also has a cancer but has a less suppressed immune response in the cancer tissue as indicated by a biomarker (such as high M2 macrophages, high expression of an immune checkpoint agent such as PD-1 or PD-L1, high expression level of PLA2G2D).

In some embodiments, the cancer has a decreased number (such as a decrease by at least 5%, 10%, 15%, 20%, 25%, 30%, 40%, 50%, 60%, 70%, 80%, 90%, or 95%) of immune cells (such as activated T cells, activated CD4+ T cells, or activated CD8+ T cells) in the cancer tissue as compared to that of a reference tissue. In some embodiments, the cancer has a decreased number (such as a decrease by at least 5%, 10%, 15%, 20%, 25%, 30%, 40%, 50%, 60%, 70%, 80%, 90%, or 95%) of activated immune cells (such as activated T cells, activated CD4+ T cells, or activated CD8+ T cells) in the cancer tissue as compared to that of a reference tissue. In some embodiments, the cancer tissue has a decreased level (such as a decrease by at least 5%, 10%, 15%, 20%, 25%, 30%, 40%, 50%, 60%, 70%, 80%, 90%, or 95%) of a cytokine (such as a pro-inflammatory cytokine, such as IFNγ or IL-2) as compared to that of a reference tissue.

In some embodiments, the reference tissue is the corresponding tissue in a healthy individual. In some embodiments, the reference tissue is the corresponding tissue in an individual who also has a cancer but has a less suppressed immune response in the cancer tissue. The suppression of immune response can be assessed by measuring a) the number of immune cells (e.g., CD3+ cells); b) the proliferating/expanding status of immune cells; c) the activation status of immune cells; and/or d) the cytokine level. In some embodiments, any one or more of the a)-d) is measured in the cancer tissue. In some embodiments, the immune cells are T cells. In some embodiments, the immune cells are CD8+ T cells (such as activated CD8+ T cells). In some embodiments, the immune cells are CD4+ T cells (such as activated CD4+ T cells).

Examples of cancers that may be treated by the methods of this application include, but are not limited to, anal cancer, astrocytoma (e.g., cerebellar and cerebral), basal cell carcinoma, bladder cancer, bone cancer, (osteosarcoma and malignant fibrous histiocytoma), brain tumor (e.g., glioma, brain stem glioma, cerebellar or cerebral astrocytoma (e.g., astrocytoma, malignant glioma, medulloblastoma, and glioblastoma), breast cancer, cervical cancer, colon cancer, colorectal cancer, endometrial cancer (e.g., uterine cancer), esophageal cancer, eye cancer (e.g., intraocular melanoma and retinoblastoma), gastric (stomach) cancer, gastrointestinal stromal tumor (GIST), head and neck cancer, hepatocellular (liver) cancer (e.g., hepatic carcinoma and heptoma), liver cancer, lung cancer (e.g., small cell lung cancer, non-small cell lung cancer, adenocarcinoma of the lung, and squamous carcinoma of the lung), medulloblastoma, melanoma, mesothelioma, myelodysplastic syndromes, nasopharyngeal cancer, neuroblastoma, ovarian cancer, pancreatic cancer, parathyroid cancer, cancer of the peritoneal, pituitary tumor, rectal cancer, renal cancer, renal pelvis and ureter cancer (transitional cell cancer), rhabdomyosarcoma, skin cancer (e.g., non-melanoma (e.g., squamous cell carcinoma), melanoma, and Merkel cell carcinoma), small intestine cancer, squamous cell cancer, testicular cancer, thyroid cancer, and tuberous sclerosis. Additional examples of cancers can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals): and SEER Program Coding and Staging Manual 2016, each of which are incorporated by reference in their entirety for all purposes.

In some embodiments, the disease or condition is a colon cancer.

In some embodiments, the disease or condition is melanoma.

In some embodiments, the disease or condition is a T cell lymphoma.

### Infectious Disease

In some embodiments, the disease or condition is an infectious disease. In some embodiments, the infectious disease is a viral infectious disease.

In some embodiments, the viral infectious disease is characterized by infection with hepatitis virus, human immunodeficiency virus (HIV), picomavirus, poliovirus, enterovirus, human Coxsackie virus, influenza virus, rhinovirus, echovirus, rubella virus, encephalitis virus, rabies virus, herpes virus, papillomavirus, polyoma virus, RSV, adenovirus, yellow fever virus, dengue virus, parainfluenza virus, hemorrhagic virus, pox virus, varicella zoster virus, parainfluenza virus, reovirus, orbivirus, rotavirus, parvovirus, African swine fever virus, measles, coronavirus (such as SARS-CoV, MERS-CoV, 2019-nCoV), Ebola virus, mumps or Norwalk virus. In some embodiments, the viral infectious disease is characterized by infection with an oncogenic virus such as CMV, EBV, HBV, KSHV, HPV, MCV, HTLV-1, HIV-1, or HCV. In some embodiments, the one or more genes encoding proteins involved in the viral infectious disease development and/or progression include, but are not limited to, genes encoding RSV nucleocapsid, Pre-gen/Pre-C, Pre-S1, Pre-S2/S,X, HBV conserved sequences, HIV Gag polyprotein (p55), HIV Pol polyprotein, HIV Gag-Pol precursor (p160W), HIV matrix protein (MA, p17), HIV capsid protein (CA, p24). HIV spacer peptide 1 (SP1, p2), HIV nucleocapsid protein (NC, p9), HIV spacer peptide 2 (SP2, p1), HIV P6 protein, HIV reverse transcriptase (RT, p50), HIV RNase H (p15), HIV integrase (IN, p31), HIV protease (PR, p10), HIV Env (gp160), gp120, gp41, HIV transactivator (Tat), HIV regulator of expression of virion proteins (Rev), HIV lentivirus protein R (Vpr). HIV Vif, HIV negative factor (Nef), HIV virus protein U (Vpu), human CCR5, miR-122, EBOV polymerase L, VP24, VP40, GP/sGP, VP30, VP35, NPC1, and TIM-1, including mutants thereof.

In some embodiments, the viral infectious disease is characterized by infection with coronavirus. In some embodiments, the viral infectious disease is characterized by infection with influenza virus.

An infection site refers to a tissue in the body where virus appear in a significant number and/or causes significant damages. In some embodiments, the infection site comprises has an increased expression level of PLA2G2D as compared to a reference tissue. In some embodiments, the PLA2G2D expression level in the infection site is increased by at least about 10%, 20%, 30%, 40%, 50%, 60%, 70%, 80%, 90% as compared to that of the reference tissue. In some embodiments, the PLA2G2D expression level in the infection site is increased by at least about 1-fold, 2-fold, 3-fold, 4-fold, or 5-fold as compared to that of the reference tissue.

In some embodiments, the infection site has a decreased number (such as a decrease by at least 5%, 10%, 15%, 20%, 25%, 30%, 40%, 50%, 60%, 70%, 80%, 90%, or 95%) of immune cells (such as activated T cells, activated CD4+ T cells, or activated CD8+ T cells) in the infection site as compared to that of a reference tissue. In some embodiments, the infection site has a decreased number (such as a decrease by at least 5%, 1(0%, 15%, 20%, 25%, 30%, 40%, 50%, 60%, 70%, 80%, 90%, or 95%) of activated immune cells (such as activated T cells, activated CD4+ T cells, or activated CD8+ T cells) in the infection site as compared to that of a reference tissue. In some embodiments, the infection site has a decreased level (such as a decrease by at least 5%, 10%, 15%, 20%, 25%, 30%, 40%, 50%, 60%, 70%, 80%, 90%, or 95%) of a cytokine (such as a pro-inflammatory cytokine, such as IFNγ or IL-2) as compared to that of a reference tissue.

In some embodiments, the reference tissue is the corresponding tissue in a healthy individual. In some embodiments, the reference tissue is the corresponding tissue in an individual who also has a virus infection (such as a virus infection of the same type) but has a less suppressed immune response in the infection site. The suppression of immune response can be assessed by measuring a) the number of immune cells; b) the proliferating/expanding status of immune cells: c) the activation status of immune cells; and/or d) the cytokine level. In some embodiments, the immune cells in circulation are assessed. In some embodiments, the immune cells in diseased tissue are assessed. In some embodiments, the immune cells in lymph tissue (such as lymph node) are assessed. In some embodiments, the immune cells are T cells. In some embodiments, the immune cells are CD8+ T cells (such as activated CD8+ T cells). In some embodiments, the immune cells are CD4+ T cells (such as activated CD4+ T cells).

### Individual

In some embodiments, the individual is a mammal (such as a human).

In some embodiments, the individual is selected for treatment based upon a high expression of PLA2G2D in a diseased tissue. In some embodiments, the tissue is a cancer tissue. In some embodiments, the tissue is an infection site.

In some embodiments, the PLA2G2D expression level in the infection site is increased by at least about 10%, 20%, 30%, 40%, 50%, 60%, 70%, 80%, 90% as compared to that of the reference tissue. In some embodiments, the PLA2G2D expression level in the infection site is increased by at least about 1-fold, 2-fold, 3-fold, 4-fold, or 5-fold as compared to that of the reference tissue.

In some embodiments, the individual is selected for treatment based upon the indication of a suppressed immune response. In some embodiments, the individual has a suppressed immune response in a diseased tissue. In some embodiments, the tissue is a cancer tissue. In some embodiments, the tissue is an infection site.

As described above, the suppression of immune response can be assessed by measuring a) the number of immune cells; b) the proliferating/expanding status of immune cells; c) the activation status of immune cells: and/or d) the cytokine level. In some embodiments, the immune cells in circulation are assessed. In some embodiments, the immune cells in diseased tissue are assessed. In some embodiments, the immune cells in lymph tissue (such as lymph node) are assessed. In some embodiments, the immune cells are T cells. In some embodiments, the immune cells are CD8+ T cells (such as activated CD8+ T cells). In some embodiments, the immune cells are CD4+ T cells (such as activated CD4+ T cells).

In some embodiments, the individual has a decreased number (such as a decrease by at least 5%, 10%, 15%, 20%, 25%, 30%, 40%, 50%, 60%, 70%, 80%, 90%, or 95%) of immune cells (such as activated T cells, activated CD4+ T cells, or activated CD8+ T cells) in the tissue (such as the cancer tissue or infection site) as compared to that of a reference tissue. In some embodiments, the individual has a decreased number (such as a decrease by at least 5%, 10%, 15%, 20%, 25%, 30%, 40%, 50%, 60%, 70%, 80%, 90%, or 95%) of activated immune cells (such as activated T cells, activated CD4+ T cells, or activated CD8+ T cells) in the tissue (such as the cancer tissue or infection site) as compared to that of a reference tissue. In some embodiments, the individual has a decreased level (such as a decrease by at least 5%, 10%, 15%, 20%, 25%, 30%, 40%, 50%, 60%, 70%, 80%, 90%, or 95%) of a cytokine (such as a pro-inflammatory cytokine, such as IFNγ or IL-2) in the tissue (such as the cancer tissue or infection site) as compared to that of a reference tissue.

In some embodiments, the reference tissue is the corresponding tissue in a healthy individual. In some embodiments, the reference tissue is the corresponding tissue in an individual who also has a same or similar disease or condition but has a less suppressed immune response in the cancer tissue.

In some embodiments, the individual has a compromised immune system.

In some embodiments, the individual is at least about 60, 65, 70, 75, 80, 85, or 90 years old.

In some embodiments, the individual has at least one prior therapy. In some embodiments, the prior therapy comprises a radiation therapy, a chemotherapy and/or an immunotherapy. In some embodiments, the individual is resistant, refractory, or recurrent to the prior therapy.

### Combination Therapy

The present application also provides methods administering an effective amount of an antagonist targeting PLA2G2D signaling pathway into an individual for treating a disease or condition (such as cancer or infectious disease), wherein the method further comprises administering a second agent or therapy. In some embodiments, the second agent or therapy is a standard or commonly used agent or therapy for treating the disease or condition.

In some embodiments, the antagonist is administered simultaneously with the second agent or therapy. In some embodiments, the antagonist is administered concurrently with the second agent or therapy. In some embodiments, the antagonist is administered sequentially with the second agent or therapy.

### Exemplary Combination Therapies for Cancer

In some embodiments, the second agent or therapy comprises a chemotherapeutic agent. In some embodiments, the second agent or therapy comprises a surgery. In some embodiments, the second agent or therapy comprises a radiation therapy. In some embodiments, the second agent or therapy comprises an immunotherapy. In some embodiments, the second agent or therapy comprises a cell therapy (such as a cell therapy comprising an immune cell (e.g., CAR T cell)). In some embodiments, the second agent or therapy comprises an angiogenesis inhibitor.

In some embodiments, the second agent is selected from the group consisting of a chemotherapeutic agent, an immunomodulator, an anti-angiogenesis agent, a growth inhibitory agent, and an antineoplastic agent.

In some embodiments, the second agent is a chemotherapeutic agent. In some embodiments, the second agent is antimetabolite agent. In some embodiments, the antimetabolite agent is 5-FU.

In some embodiments, the second agent is an immunomodulator. In some embodiments, the immunomodulatory is an immune checkpoint inhibitor. In some embodiments, the checkpoint inhibitor specifically targets PD-L1, PD-L2, CTLA4, PD-L2. PD-1, CD47, TIGIT, GITR, TIM3, LAG3, CD27, 4-1BB, or B7H4. In some embodiments, the second agent is an anti-PD-1 antibody or fragment thereof. In some embodiments, the second agent is an anti-PD-L1 antibody or fragment thereof.

In some embodiments, the second agent comprises a cell (such as an immune cell, such as a T cell) comprising a chimeric antigen receptor that specifically binds to a tumor antigen.

### Exemplary Combination Therapies for Infectious Disease (Such as Viral Infectious Disease).

In some embodiments, the second agent or therapy comprises a nucleotide analogue.

In some embodiments, the second agent or therapy comprises a nucleoside analogue.

In some embodiments, the second agent or therapy comprises a protease inhibitor. In some embodiments, the second agent or therapy comprises Lopinavir. In some embodiments, the second agent or therapy comprises ritonavir.

In some embodiments, the second agent or therapy comprises a neuraminidase inhibitor. In some embodiments, the second agent or therapy comprises zanamivir. In some embodiments, the second agent or therapy comprises oseltamivir. In some embodiments, the second agent or therapy comprises peramivir.

In some embodiments, the second agent or therapy comprises a Cap-dependent endonuclease inhibitor. In some embodiments, the second agent or therapy comprises baloxavir.

In some embodiments, the second agent or therapy comprises a sialidase.

The second agent and the antagonist can be administered sequentially, concurrently, or simultaneously. In some embodiments, the second agent is administered prior to the antagonist. In some embodiments, the second agent is administered after the antagonist.

### Dosing Regimen and Routes of Administration

The dose of the antagonist and, in some embodiments, the second agent as described herein, administered to an individual (such as a human) may vary with the particular composition, the method of administration, and the particular kind and stage of disease or condition being treated. The amount should be sufficient to produce a desirable response, such as a therapeutic response against the disease or condition. In some embodiments, the amount of the antagonist and/or the second agent is a therapeutically effective amount.

In some embodiments, the amount of the antagonist is an amount sufficient to decrease the suppression of the immune response in the individual. Whether there is a decrease in the suppression of the immune response and the extent of the decrease in suppression can be indicated by any of the following.

In some embodiments, the amount of the antagonist is an amount sufficient to increase the number of immune cells (such as T cells, such as CD4+ and/or CD8+ T cells) by at least about 10%, 20%, 30%, 40%, 50%, 60%, 70%, 80%, 90%, or 100% post administration of the antagonist. In some embodiments, the immune cells in circulation are assessed. In some embodiments, the immune cells in diseased tissue are assessed. In some embodiments, the immune cells in lymph tissue (such as lymph node) are assessed. In some embodiments, the immune cells comprises myeloid cells (such as dendritic cells). In some embodiments, the immune cells comprises NK cells. In some embodiments, the immune cells comprises T cells, such as CD4+ and/or CD8+ T cells. In some embodiments, the number of immune cells is assessed about 1, 2, 3, 4, 5, 6, or 7 days post administration of the antagonist.

In some embodiments, the amount of the antagonist is an amount sufficient to increase the number of activated immune cells (such as activated CD4+ and/or CD8+ T cells) by at least about 10%, 20%, 30%, 40%, 50%, 60%, 70%, 80%, 90%, or 100% post administration of the antagonist. In some embodiments, the activated immune cells in circulation are assessed. In some embodiments, the activated immune cells in diseased tissue are assessed. In some embodiments, the activated immune cells in lymph tissue (such as lymph node) are assessed. In some embodiments, the immune cells comprises myeloid cells (such as dendritic cells). In some embodiments, the immune cells comprises NK cells. In some embodiments, the immune cells comprises T cells, such as CD4+ and/or CD8+ T cells. In some embodiments, the number of activated immune cells is assessed about 1, 2, 3, 4, 5, 6, or 7 days post administration of the antagonist.

In some embodiments, the amount of the antagonist is an amount sufficient to increase the proliferation of immune cells or activated immune cells by at least about 10%, 20%, 30%, 40%, 50%, 60%, 70%, 80%, 900%, or 100% post administration of the antagonist. In some embodiments, the immune cells or the activated immune cells in circulation are assessed. In some embodiments, the immune cells or the activated immune cells in diseased tissue are assessed. In some embodiments, the immune cells or the activated immune cells in lymph tissue (such as lymph node) are assessed. In some embodiments, the immune cells comprises myeloid cells (such as dendritic cells). In some embodiments, the immune cells comprises NK cells. In some embodiments, the immune cells comprises T cells, such as CD4+ and/or CD8+ T cells. In some embodiments, the proliferation of immune cells or activated immune cells is assessed about 1, 2, 3, 4, 5, 6, or 7 days post administration of the antagonist.

In some embodiments, the amount of the antagonist is an amount sufficient to increase the cytokine level (such as a pro-inflammatory cytokine, such as IFNγ or IL-2) by at least about 10%, 20%, 30%, 40%, 50%, 60%, 70%, 80%, 90%, or 100% post administration of the antagonist. In some embodiments, the cytokine level in diseased tissue is assessed. In some embodiments, the level of cytokine is assessed about 1, 2, 3, 4, 5, 6, or 7 days post administration of the antagonist.

In some embodiments, the amount of the antagonist is an amount sufficient to decrease the suppressive immune cells by at least about 10%, 20%, 30%, 40%, 50%, 60%, 70%, 80%, or 90% post administration. In some embodiments, the suppressive immune cells comprise regulatory T cells. In some embodiments, the suppressive immune cells comprise myeloid derived suppressor cells. In some embodiments, the suppressive immune cells in circulation are assessed. In some embodiments, the suppressive immune cells in diseased tissue are assessed. In some embodiments, the suppressive immune cells in lymph tissue (such as lymph node) are assessed. In some embodiments, the number of suppressive immune cells is assessed about 1, 2, 3, 4, 5, 6, or 7 days post administration of the antagonist.

In some embodiments, the amount of the antagonist is an amount sufficient to increase humoral immune response in the individual by at least about 10%, 20%, 30%, 40%, 50%, 60%, 70%, 80%, 90%, or 100% post administration of the antagonist. The humoral immune response can be assessed by measuring antibodies (such as IgG antibodies) that target a disease-associated antigen or plasmablasts that produce such antibodies in circulation. In some embodiments, the humoral immune response is assessed about 7-28 days (such as about 7-14 days) post administration of the antagonist.

In some embodiments, the amount of the antagonist is an amount sufficient to produce a decrease of the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor size, number of cancer cells, or tumor growth rate in the same individual prior to treatment or compared to the corresponding activity in other individuals not receiving the treatment.

In some embodiments, the antagonist is administered at a dose of about 0.001 μg/kg to about 100 mg/kg of total body weight, for example, about 0.005 μg/kg to about 50 mg/kg, about 0.01 sg/kg to about 10 mg/kg, or about 0.01 μg/kg to about 1 mg/kg.

In some embodiments according to any one of the methods described herein, the antagonist and/or the second agent composition is administered intravenously, intraarterially, intraperitoneally, intravesicularlly, subcutaneously, intrathecally, intrapulmonarily, intramuscularly, intratracheally, intraocularly, topically, transdermally, orally, or by inhalation. In some embodiments, the antagonist and/or the second agent is administered intravenously.

In some embodiments, the antagonist is administered directly to the diseased tissue.

### III. METHODS OF DIAGNOSIS AND PROGNOSIS

Provided herein also include methods of diagnosing or prognosing an individual, including, determining the suitability of an individual for the treatment as described in section II or a different therapy comprising an immunotherapy, determining the likelihood of responsiveness of an individual to the methods as described in section II or the different therapy.

In some embodiments, there is provided a method of determining the suitability of an individual for a treatment, comprising measuring levels of PLA2G2D expression in the diseased tissue of an individual. In some embodiments, the individual has a cancer, and the tissue is a tumor tissue. In some embodiments, the individual has an infectious disease (such as a viral infectious disease, and the tissue is an infection site.

In some embodiments, there is provided a method of prognosis in an individual having cancer (such as a solid tumor), comprising measuring levels of PLA2G2D expression in a tumor sample in vitro or in vivo, wherein a higher PLA2G2D expression level as compared to a reference level indicates a higher possibility of not responding or responding poorly to a therapy (such as an immunotherapy). In some embodiments, the reference level is a level of PLA2G2D expression (such as an average PLA2G2D expression) in a non-tumor sample in the individual or a corresponding tissue in a different individual (or a group of individuals) who does not have cancer.

In some embodiments, there is provided a method of prognosis in an individual having an infectious disease (such as a virus infectious disease), comprising measuring levels of PLA2G2D expression in a sample from the infection site in vitro or in vivo, wherein a higher PLA2G2D expression level as compared to a reference level indicates a higher possibility of not responding or responding poorly to a therapy (such as an immunotherapy).

In some embodiments, the reference level is a level of PLA2G2D expression (such as an average PLA2G2D expression) in a non-infection site sample in the individual or a corresponding tissue in a different individual (or a group of individuals) who does not have the infectious disease.

In some embodiments, the therapy comprises a cell therapy (such as a CAR-T cell therapy).

In some embodiments, the therapy further comprises assessing suppression of immune response in the individual. Exemplary methods of assessing immune response suppression are discussed above.

### IV. METHODS OF PREPARATION, NUCLEIC ACIDS, VECTORS, HOST CELLS, AND CULTURE MEDIUM

In some embodiments, there is provided a method of preparing the antagonist (such as an siRNA targeting PLA2G2D, anti-PLA2G2D agents, inhibitory PLA2G2D polypeptides, agents inhibiting PLA2G2D enzymatic activity as described herein) and composition comprising the agents, nucleic acid construct, vector, host cell, or culture medium that is produced during the preparation of the agents.

### Polypeptide Expression and Production

The agents targeting PLA2G2D (e.g., polypeptide comprising an anti-PLA2G2D antibody moiety as described in Section ii) and inhibitory PLA2G2D polypeptides described herein can be prepared using any known methods in the art, including those described below.

### Polypeptides Comprising Anti-PLA2G2D Antibody Moiety

Monoclonal antibodies targeting PLA2G2D can be obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies. For example, the monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster or a llama, is immunized as hereinabove described to elicit lymphocytes that produce or are capable of producing antibodies that will specifically bind the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice. pp. 59-103 (Academic Press, 1986).

The immunizing agent will typically include the antigenic protein or a fusion variant thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice, Academic Press (1986), pp. 59-103, incorporated by reference in its entirety for all purposes.

Immortalized cell lines are usually transformed mammalian cells, particularly myeloma cells of rodent, bovine and human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors available from the Salk Institute Cell Distribution Center, San Diego, Calif. USA, and SP-2 cells (and derivatives thereof, e.g., X63-Ag8-653) available from the American Type Culture Collection, Manassas, Va. USA. Human myeloma and mouse-human heteromyeloma cell lines also have been described for the production of human monoclonal antibodies (Kozbor, J. Immunol., 133:3001 (1984): Brodeur et al., Monoclonal Antibody Production Techniques and Applications, pp. 51-63 (Marcel Dekker, Inc., New York, 1987), each of which are incorporated by reference in their entirety for all purposes).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed against the desired antigen. Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5:256-262 (1993) and Pluckthun, Immunol. Revs. 130:151-188 (1992).

In a further embodiment, antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991), each of which are incorporated by reference in their entirety for all purposes, describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567: Morrison, et al., Proc. Natl Acad. Sci. USA, 81:6851 (1984)), or by covalently joining to the immunoglobulin coding sequence all or part of the coding sequence for a non-immunoglobulin polypeptide. Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### Nucleic Acid Molecules Encoding Polypeptides

In some embodiments, there is provided a polynucleotide encoding any one of the antibodies (such as anti-PLA2G2D antibodies) or polypeptides (such as inhibitory PLA2G2D polypeptides) described herein. In some embodiments, there is provided a polynucleotide prepared using any one of the methods as described herein. In some embodiments, a nucleic acid molecule comprises a polynucleotide that encodes a heavy chain or a light chain of an antibody (e.g., anti-PLA2G2D antibody). In some embodiments, a nucleic acid molecule comprises a polynucleotide that encodes an inhibitory PLA2G2D polypeptide. In some embodiments, a nucleic acid molecule comprises both a polynucleotide that encodes a heavy chain and a polynucleotide that encodes a light chain, of an antibody (e.g., anti-PLA2G2D antibody). In some embodiments, a first nucleic acid molecule comprises a first polynucleotide that encodes a heavy chain and a second nucleic acid molecule comprises a second polynucleotide that encodes a light chain. In some embodiments, a nucleic acid molecule encoding a scFv (e.g., anti-PLA2G2D scFv) is provided. In some embodiments, a nucleic acid molecule comprises a polynucleotide that encodes an inhibitory PLA2G2D polypeptide.

In some such embodiments, the heavy chain and the light chain of an antibody (e.g., anti-PLA2G2D antibody) are expressed from one nucleic acid molecule, or from two separate nucleic acid molecules, as two separate polypeptides. In some embodiments, such as when an antibody is a scFv, a single polynucleotide encodes a single polypeptide comprising both a heavy chain and a light chain linked together.

In some embodiments, a polynucleotide encoding a heavy chain or light chain of an antibody (e.g., anti-PLA2G2D antibody) comprises a nucleotide sequence that encodes a leader sequence, which, when translated, is located at the N terminus of the heavy chain or light chain. As discussed above, the leader sequence may be the native heavy or light chain leader sequence, or may be another heterologous leader sequence.

In some embodiments, the polynucleotide is a DNA. In some embodiments, the polynucleotide is an RNA. In some embodiments, the RNA is an mRNA.

Nucleic acid molecules may be constructed using recombinant DNA techniques conventional in the art. In some embodiments, a nucleic acid molecule is an expression vector that is suitable for expression in a selected host cell.

### Nucleic Acid Construct

In some embodiments, there is provided a nucleic acid construct comprising any one of the polynucleotides described herein. In some embodiments, there is provided a nucleic acid construct prepared using any method described herein.

In some embodiments, the nucleic acid construct further comprises a promoter operably linked to the polynucleotide. In some embodiments, the polynucleotide corresponds to a gene, wherein the promoter is a wild-type promoter for the gene.

### Vectors

The terms “vector”, “cloning vector” and “expression vector” mean the vehicle by which a DNA or RNA sequence (e.g., a foreign gene) can be introduced into a host cell, so as to genetically modify the host and promote expression (e.g., transcription and translation) of the introduced sequence. Vectors include plasmids, synthesized RNA and DNA molecules, phages, viruses, etc. In certain embodiments, the vector is a viral vector such as, but not limited to, viral vector is an adenoviral, adeno-associated, alphaviral, herpes, lentiviral, retroviral, or vaccinia vector.

In some embodiments, there is provided a vector comprising any polynucleotides that encode the heavy chains and/or light chains of any one of the antibodies (e.g., anti-PLA2G2D antibodies) described herein. In some embodiments, there is provided a vector comprising any polynucleotides that encode polypeptides (e.g., inhibitory PLA2G2D polypeptides) described herein. In some embodiments, there is provided a vector comprising any nucleic acid construct described herein. In some embodiments, there is provided a vector prepared using any method described herein. Vectors comprising polynucleotides that encode any of polypeptides (such as anti-PLA2G2D antibodies or inhibitory PLA2G2D polypeptides) are also provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides.

In some embodiments, a first vector comprises a polynucleotide that encodes a heavy chain of an antibody (e.g., anti-PLA2G2D antibody) and a second vector comprises a polynucleotide that encodes a light chain of an antibody (e.g., anti-PLA2G2D antibody). In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, e.g., in Running Deer et al., Biotechnol. Prog. 20:880-889 (2004).

In certain embodiments, the vector is a viral vector. In certain embodiments, the viral vector can be, but is not limited to, a retroviral vector, an adenoviral vector, an adeno-associated virus vector, an alphaviral vector, a herpes virus vector, and a vaccinia virus vector. In some embodiments, the viral vector is a lentiviral vector.

In some embodiments, the vector is a non-viral vector. The viral vector may be a plasmid or a transposon (such as a PiggyBac- or a Sleeping Beauty transposon).

### Host Cells

In some embodiments, there is provided a host cell comprising any polypeptide, nucleic acid construct and/or vector described herein. In some embodiments, there is provided a host cell prepared using any method described herein. In some embodiments, the host cell is capable of producing any of polypeptides (such as antibodies or inhibitory polypeptides) described herein under a fermentation condition.

In some embodiments, the polypeptides described herein (e.g., anti-PLA2G2D antibodies or inhibitory PLA2G2D polypeptides) may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells (Crucell); and NSO cells. In some embodiments, the polypeptides described herein (e.g., anti-PLA2G2D antibodies or inhibitory PLA2G2D polypeptides) may be expressed in yeast. See, e.g., U.S. Publication No. US 2006/0270045 A1. In some embodiments, a particular eukaryotic host cell is selected based on its ability to make desired post-translational modifications to the heavy chains and/or light chains of the desired antibody. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Non-limiting exemplary methods are described, e.g., in Sambrook et al., Molecular Cloning, A Laboratory Manual, 3rd ed. Cold Spring Harbor Laboratory Press (2001), incorporated by reference in its entirety for all purposes. Nucleic acids may be transiently or stably transfected in the desired host cells, according to any suitable method.

The invention also provides host cells comprising any of the polynucleotides or vectors described herein. In some embodiments, the invention provides a host cell comprising an anti-PLA2G2D antibody. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

In some embodiments, the polypeptide is produced in a cell-free system. Non-limiting exemplary cell-free systems are described, e.g., in Sitaraman et al., Methods Mol. Biol. 498: 229-44 (2009): Spirin, Trends Biotechnol. 22: 53845 (2004); Endo et al., Biotechnol. Adv. 21: 695-713 (2003).

### Purification of Polypeptides

The polypeptides (e.g., anti-PLA2G2D antibodies, e.g., inhibitory PLA2G2D polypeptides) may be purified by any suitable method. Such methods include, but are not limited to, the use of affinity matrices or hydrophobic interaction chromatography. Suitable affinity ligands include the ROR1 ECD and ligands that bind antibody constant regions. In some embodiments, a Protein A, Protein G, Protein A/G, or an antibody affinity column may be used to bind the constant region and to purify an antibody comprising an Fc fragment. Hydrophobic interactive chromatography, for example, a butyl or phenyl column, may also suitable for purifying some polypeptides such as antibodies. Ion exchange chromatography (e.g. anion exchange chromatography and/or cation exchange chromatography) may also suitable for purifying some polypeptides such as antibodies. Mixed-mode chromatography (e.g. reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

### V. COMPOSITIONS, KITS, AND ARTICLES OF MANUFACTURE

The present application also provides compositions, kits, medicines, and unit dosage forms for use in any of the methods described herein.

### Compositions

Any of the antagonists described herein can be present in a composition (such as a formulation) that includes other agents, excipients, or stabilizers.

In some embodiments, the composition further comprises a target agent or a carrier that promotes the delivery of the antagonist to a diseased tissue. Exemplary carriers include liposomes, micelles, nanodisperse albumin and its modifications, polymer nanoparticles, dendrimers, inorganic nanoparticles of different compositions.

In some embodiments, the antagonist is packaged in a nanocarrier. In some embodiments, the nanocarrier has an average diameter of about 20 nm to about 200 nm. In some embodiments, the nanocarrier has an average diameter of about 50 nm.

In some embodiments, the antagonist described herein is coated with a serum protein (such as albumin). In some embodiments, the antagonist is coated with opsonin.

In some embodiments, the antagonist comprises or is coupled with a moiety that facilitate the delivery of the antagonist to the diseased tissue (such as the cancer tissue or infection site as described above). In some embodiments, the moiety bind to an antigen that is expressed (e.g., overexpressed) or clustered on the diseased tissue (such as cancer tissue) or cells within the diseased tissue. In some embodiments, the antigen is a tumor associated antigen (such as Her2, folate receptor, CD44). See, for example, Rosenblum et al., Nat Commun. 2018 Apr. 12; 9(1):1410.

In some embodiments, the composition is suitable for administration to a human. In some embodiments, the composition is suitable for administration to a mammal such as, in the veterinary context, domestic pets and agricultural animals. There are a wide variety of suitable formulations of the composition comprising the antagonist. The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, poly vinylpyrrolidone, cellulose, water, saline solution, syrup, methylcellulose, methyl- and propylhydroxybenzoates, talc, magnesium stearate, and mineral oil. In some embodiments, the composition comprising the antagonist with a carrier as discussed herein is present in a dry formulation (such as lyophilized composition). The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the composition is formulated to have a pH range of about 4.5 to about 9.0, including for example pH ranges of about any of 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

### Kits

Kits provided herein include one or more containers comprising the antagonist or a pharmaceutical composition comprising the antagonist described herein and/or other agent(s), and in some embodiments, further comprise instructions for use in accordance with any of the methods described herein. The kit may further comprise a description of selection of individual suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

In some embodiments, the kit comprises a) a composition comprising an antagonist targeting PLA2G2D signaling pathway comprising an agent comprising an anti-PLA2G2D antibody moiety, or a pharmaceutically acceptable salt thereof and a pharmaceutically acceptable carrier: and optionally b) instructions for administering the agent for treatment of a disease or condition. In some embodiments, the agent is an anti-PLA2G2D antibody. In some embodiments, the agent is an anti-PLA2G2D fusion protein. In some embodiments, the agent is an anti-PLA2G2D immunoconjugate.

In some embodiments, the kit comprises a) a composition comprising an antagonist targeting PLA2G2D signaling pathway comprising an inhibitory PLA2G2D polypeptide, or a pharmaceutically acceptable salt thereof and a pharmaceutically acceptable carrier; and optionally b) instructions for administering the agent for treatment of a disease or condition. In some embodiments, the inhibitory PLA2G2D polypeptide comprises an amino acid sequence selected from the group consisting of SEQ ID NOs: 3, 4, and 7-12.

In some embodiments, the kit comprises a) a composition comprising an antagonist targeting PLA2G2D signaling pathway comprising a nucleic acid agent targeting PLA2G2D (such as siRNA, shRNA, miRNA, or antisense RNA), or a pharmaceutically acceptable salt thereof and a pharmaceutically acceptable carrier; and optionally b) instructions for administering the agent for treatment of a disease or condition.

In some embodiments, the kit comprises a) a composition comprising an antagonist targeting PLA2G2D signaling pathway comprising a genome editing system that targets PLA2G2D, or a pharmaceutically acceptable salt thereof and a pharmaceutically acceptable carrier: and optionally b) instructions for administering the agent for treatment of a disease or condition.

In some embodiments, the kit comprises a) a composition comprising an antagonist targeting PLA2G2D signaling pathway comprising an agent inhibiting PLA2G2D enzymatic activity, or a pharmaceutically acceptable salt thereof and a pharmaceutically acceptable carrier: and optionally b) instructions for administering the agent for treatment of a disease or condition.

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

In some embodiments, the kits comprise one or more components that facilitate delivery of the antagonist, or a composition comprising the agent, and/or additional therapeutic agents to the individual. In some embodiments, the kit comprises, e.g., syringes and needles suitable for delivery of cells to the individual, and the like. In such embodiments, the antagonist, or a composition comprising the agent may be contained in the kit in a bag, or in one or more vials. In some embodiments, the kit comprises components that facilitate intravenous or intra-arterial delivery of the antagonist, or a composition comprising the agent to the individual. In some embodiments, the antagonist, or a composition comprising the agent may be contained, e.g., within a bottle or bag (for example, a blood bag or similar bag able to contain up to about 1.5 L solution comprising the cells), and the kit further comprises tubing and needles suitable for the delivery of the antagonist, or a composition comprising the agent to the individual.

The instructions relating to the use of the compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of the zinc as disclosed herein to provide effective treatment of an individual for an extended period, such as any of 1 day, 2 days, 3 days, 4 days, 5 days, 6 days, 7 days, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the pharmaceutical compositions and instructions for use and packaged in quantities sufficient for storage and use in pharmacies, for example, hospital pharmacies and compounding pharmacies.

### EXEMPLARY EMBODIMENTS

Embodiment 1. A method of treating a cancer or viral infection in an individual, comprising administering into the individual an effective amount of an antagonist targeting PLA2G2D signaling pathway.

Embodiment 2. The method of embodiment 1, wherein the antagonist is an antagonist targeting PLA2G2D.

Embodiment 3. The method of embodiment 2, wherein the PLA2G2D is a human PLA2G2D.

Embodiment 4. The method of embodiment 2 or embodiment 3, wherein the antagonist decreases enzymatic activity level of PLA2G2D.

Embodiment 5. The method of embodiment 4, wherein the antagonist targeting PLA2G2D signaling pathway blocks a catalytic site on PLA2G2D.

Embodiment 6. The method of embodiment 5, wherein the antagonist targets the H67 catalytic site on a human PLA2G2D according to SEQ ID NO: 1 or 5.

Embodiment 7. The method of any one of embodiments 1-3, wherein the antagonist comprises a siRNA, an miRNA, an antisense RNA, or a gene editing system.

Embodiment 8. The method of any one of embodiments 1-3, wherein the antagonist comprises an agent that inhibits PLA2G2D (such as an agent that blocks the binding of PLA2G2D to an immune cell or an agent that inhibits the activity of PLA2G2D).

Embodiment 9. The method of embodiment 8, wherein the immune cell is a T cell.

Embodiment 10. The method of any one of embodiments 1-3, wherein the antagonist comprises an anti-PLA2G2D antibody.

Embodiment 11. The method of embodiment 10, wherein the anti-PLA2G2D antibody is a monoclonal antibody.

Embodiment 12. The method of embodiment 10, wherein the antagonist is a fusion protein further comprising a second moiety.

Embodiment 13. The method of embodiment 12, wherein the second moiety comprises a cytokine.

Embodiment 14. The method of any one of embodiments 1-3, wherein the antagonist comprises an inhibitory PLA2G2D polypeptide that blocks the binding of PLA2G2D to an immune cell.

Embodiment 15. The method of embodiment 14, wherein the inhibitory PLA2G2D polypeptide binds to the immune cell with a greater affinity than for PLA2G2D.

Embodiment 16. The method of embodiment 15, wherein the immune cells is a T cell.

Embodiment 17. The method of any one of embodiments 14-16, wherein the inhibitory PLA2G2D polypeptide further comprises a stabilizing domain.

Embodiment 18. The method of embodiment 17, wherein the stabilizing domain is an Fc domain.

Embodiment 19. The method of any one of embodiments 14-18, wherein the inhibitory PLA2G2D polypeptide has a length of about 50 to about 200 amino acids.

Embodiment 20. The method of any one of embodiments 14-19, wherein inhibitory PLA2G2D polypeptide has a mutation at the position corresponding to histidine at position 67 (H67) according to SEQ ID NO: 1 or 5.

Embodiment 21. The method of embodiment 20, wherein the inhibitory PLA2G2D poly peptide comprises an amino acid sequence of SEQ ID NO: 3, 4, 7, or 8.

Embodiment 22. The method of any one of embodiments 1-21, wherein the disease or condition is a cancer.

Embodiment 23. The method of embodiment 22, wherein the cancer is a solid tumor.

Embodiment 24. The method of embodiment 22 or embodiment 23, wherein the cancer is an advanced or malignant tumor.

Embodiment 25. The method of any one of embodiments 22-24, wherein the cancer has an increased expression level of PLA2G2D.

Embodiment 26. The method of any one of embodiments 22-25, wherein the cancer is selected from the group consisting of lung cancer, breast cancer, liver cancer, gastric cancer, cervical cancer, endometrial cancer, thyroid cancer, colorectal cancer, head and neck cancer, pancreatic cancer, renal cancer, prostate cancer, urothelial cancer, testis cancer, ovarian cancer and melanoma.

Embodiment 27. The method of any one of embodiments 1-21, wherein the disease or condition is a viral infection.

Embodiment 28. The method of embodiment 27, wherein the infection site has an increased expression level of PLA2G2D.

Embodiment 29. The method of any one of embodiments 1-28, wherein the method further comprises administering a second agent.

Embodiment 30. The method of embodiment 29, wherein the second agent is selected from the group consisting of a chemotherapeutic agent, an immunomodulator, an anti-angiogenesis agent, a growth inhibitory agent, and an antineoplastic agent.

Embodiment 31. The method of embodiment 30, wherein the second agent is an immunomodulator.

Embodiment 32. The method of embodiment 31, wherein the immunomodulator is an immune checkpoint inhibitor.

Embodiment 33. The method of embodiment 28, wherein the immune checkpoint inhibitor specifically target PD-L1. PD-L2. CTLA4, PD-L2, PD-1, CD47, TIGIT, GITR, TIM3, LAG3, CD27, 4-1BB, or B7H4.

Embodiment 34. The method of embodiment 33, wherein the second agent comprises a cell comprising a chimeric antigen receptor that specifically binds to a tumor antigen.

Embodiment 35. The method of any one of embodiments 29-34, wherein the antagonist and the second agent is administered simultaneously or concurrently.

Embodiment 36. The method of any one of embodiments 29-34, wherein the antagonist and the second agent is administered sequentially.

Embodiment 37. The method of any one of embodiments 1-36, wherein the antagonist and/or the second agent is administered parentally.

Embodiment 38. The method of any one of embodiments 22-37, wherein the antagonist is administered to the cancer tissue or infection site directly.

Embodiment 39. The method of any one of embodiments 1-38, wherein the antagonist is administered at a dose of about 0.001 μg/kg to about 100 mg/kg.

Embodiment 40. The method of any one of embodiments 22-39, wherein the individual has an increased number of immune cells in the cancer tissue or at the infection site after administration of the antagonist.

Embodiment 41. The method of embodiment 40, wherein the immune cells are T cells.

Embodiment 42. The method of embodiment 40 or embodiment 41, wherein the T cells are activated T cells.

Embodiment 43. The method of any one of embodiment 40-42, wherein the number of immune cells in the cancer tissue or at the infection site is increased by at least about 5% after administration of the antagonist.

Embodiment 44. The method of any one of embodiments 22-43, wherein immune cells in the cancer tissue or at the infection site produce an increased level of a cytokine after administration of the antagonist.

Embodiment 45. The method of embodiment 44, wherein the cytokine is IFNγ and/or IL-2.

Embodiment 46. The method of embodiment 39 or embodiment 40, wherein the level of the cytokine is increased by at least about 5% after administration of the antagonist.

### EXAMPLES

The examples below are intended to be purely exemplary of the application and should therefore not be considered to limit the invention in any way. The following examples and detailed description are offered by way of illustration and not by way of limitation.

### Example 1. Identification of PLA2G2D Signaling Pathway

To identify new signaling pathways that are involved in cancer, gene expression profiles were studied.

Specifically, the Cancer Genome Atlas (TCGA) batch-corrected RNA-seq dataset was downloaded from the National Cancer Institute's Genomic Data Commons PanCanAtlas website (https://gdc.cancer.gov/about-data/publications/pancanatlas). The pan-cancer dataset was separated into the following tumor types: Bladder urothelial carcinoma (BLCA). Breast invasive carcinoma (BRCA), COAD (Colon adenocarcinoma), ESCA (Esophageal carcinoma). Head and Neck squamous cell carcinoma (HNSC), Kidney Chromophobe (KICH), KIRC (Kidney renal clear cell carcinoma), KIRP (Kidney renal papillary cell carcinoma), LIHC (Liver hepatocellular carcinoma), Lung adenocarcinoma (LUAD). Lung squamous cell carcinoma (LUSC). Ovarian serous cystadenocarcinoma (OV), Pancreatic adenocarcinoma (PAAD), Pheochromocytoma and Paraganglioma (PCPG), Prostate adenocarcinoma (PRAD). Rectum Adenocarcinoma (READ), Prostate adenocarcinoma (SARC), Skin Cutaneous Melanoma (SKCM), Stomach adenocarcinoma (STAD), Testicular Germ Cell Tumors (TGCT), Thyroid carcinoma (THCA), Thymoma (THYM), Triple negative breast cancer (TN-BRCA), and Uterine Corpus Endometrial Carcinoma (UCEC). For each tumor type, the following analysis was performed: Genes with consistently low expression (e.g. genes with no counts in more than 80% of samples) were removed and the data was log-transformed using log 2(x+1). The Weighted gene co-expression network analysis (WGCNA) R package was then used to build a gene co-expression network and identify clusters of highly correlated genes. See Langfelder, P. & Horvath, S. WGCNA: an R package for weighted correlation network analysis. BMC Bioinformatics 9, (2008).

The cluster containing the T signature genes was extracted and gene ontology analysis was performed using the R package clusterProfiler to ensure that the cluster was enriched for T-cell related pathways. See Yu G, Wang L, Han Y. He Q (2012). “clusterProfiler: an R package for comparing biological themes among gene clusters.” OMICS: A Journal of Integrative Biology, 16(5), 284-287. doi: 10.1089/omi.2011.0118. The expression dataset was then subset to genes within this T signature genes cluster and the samples were clustered using the R package ConsensusClusterPlus using k-means clustering for the clustering algorithm, Euclidean for the distance measure, and maximum cluster number k=20. See Wilkerson, D. M, Hayes, Neil D (2010). “ConsensusClusterPlus: a class discovery tool with confidence assessments and item tracking.” Bioinformatics, 26(12), 1572-1573. The cumulative distribution function (CDF) plot and the delta area plot, which shows the relative change in area under the CDF curve comparing k and k−1, were used to identify the optimal number of clusters. The expression values of the genes within each cluster were then summed to generate a single summary value to describe each cluster. Tumors in the highest gene expression cluster were labeled ‘hot’ and tumors in the lowest gene expression cluster were labeled ‘cold’. The RNA-seq read count matrix for the tumor was then downloaded from the Google Cloud Pilot RNA-Sequencing for CCLE and TCGA project data repository (https://osf.io/gqrz9/), which contains RNA-seq data from TCGA processed using kallisto. See Tatlow, P. & Piccolo, S. R A cloud-based workflow to quantify transcript-expression levels in public cancer compendia. Scientific Reports 6, (2016). The transcript-level data was summed to the gene-level and subset to protein-coding genes. Differential expression analysis was then performed using the R package limma to compare the ‘hot’ samples to the ‘cold’ samples. See Ritchie M E, Phipson B, Wu D, Hu Y, Law C W, Shi W, Smyth G K (2015). “limma powers differential expression analyses for RNA-sequencing and microarray studies.” Nucleic Acids Research, 43(7), e47. The differential expression results were visualized using the R package EnhancedVolcano. See Blighe K, Rana S, Lewis M (2019). EnhancedVolcano: Publication-ready volcano plots with enhanced colouring and labeling. R package version 1.4.0, https://github.com/kevinblighe/EnhancedVolcano.

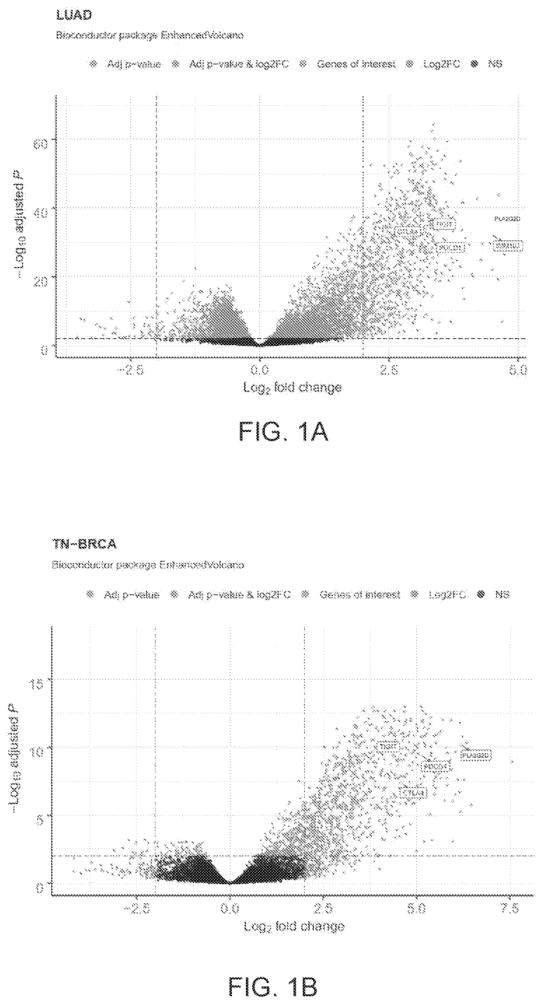

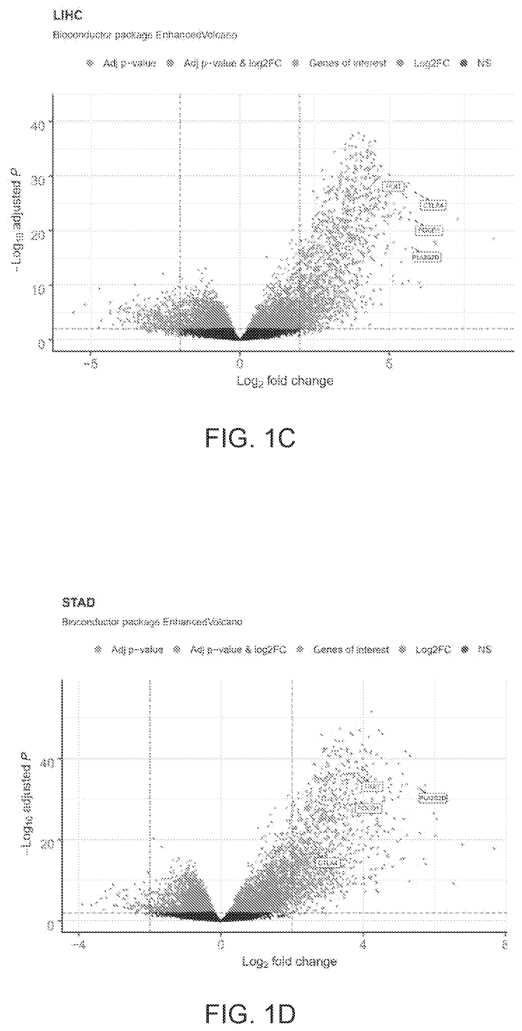

Results suggests that PLA2G2D is highly differentially expressed in four types of cancers. See FIGS. 1A-ID. Specifically and strikingly, PLA2G2D was expressed 56 times higher in CD8+ high tumors as compared to CD8+ low tumors.

### Example 2. Role of PLA2G2D in Suppression of T Cell Activation

The effect of human PLA2G2D-Fc on T cell activation was assessed in peripheral blood mononuclear cell (PBMC) or isolated T cell cultures. PBMC were isolated from leukoreduction system (LRS) chambers from healthy human donors by centrifugation over Ficoll-Paque Plus (GE Life Sciences) and labeled in a 5 μM CFSE solution (Molecular Probes) for 12 min at 37° C. and washed. 2×10⁵ labeled PBMC per well of a 96 well round bottom plate were then stimulated with 1 μg/ml anti-CD3 (OKT3, Invitrogen) and 0.2 μg/ml anti-CD28 (CD28.2, Invitrogen) in the presence of soluble 0-20 μg/ml human PLA2G2D-Fc or control human IgG1-Fc protein (Sino Biological) as indicated in a final volume of 200 μl RPMI (Corning). PBMC cultures were incubated for 72 hours at 37° C. before the supernatants were harvested and measured for IFNγ and IL-2 levels using MSD V-plex assays (Meso Scale Discovery). T cell proliferation within the PBMC culture was simultaneously assessed by staining cells with fluorophore-conjugated anti-CD3, anti-CD4, and anti-CD8 antibodies (Biolegend) and Live/Dead Fixable dead cell stain (Molecular Probes), and running on a BD LSRFortessa X-20 flow cytometer (Becton Dickinson). FACS data was analyzed using FlowJo software.

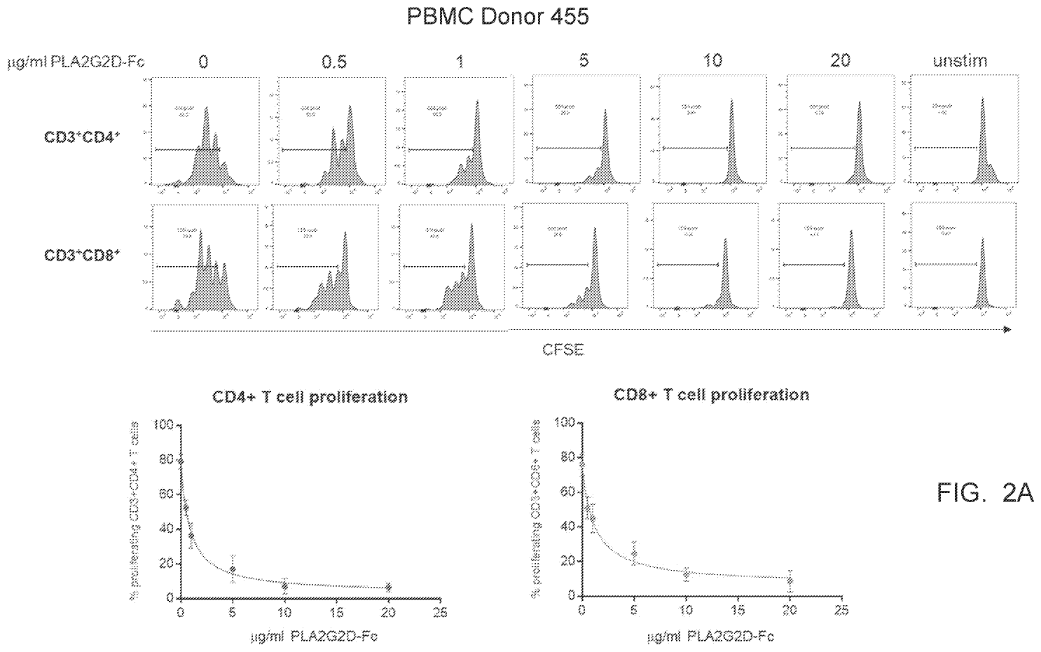

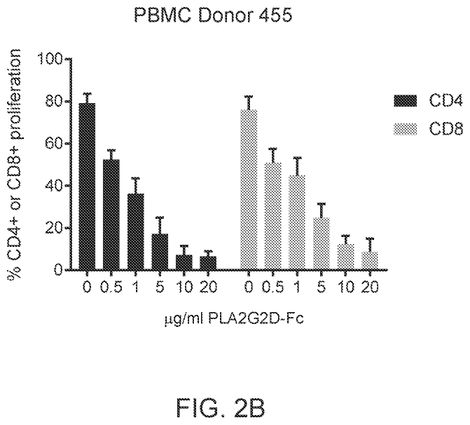

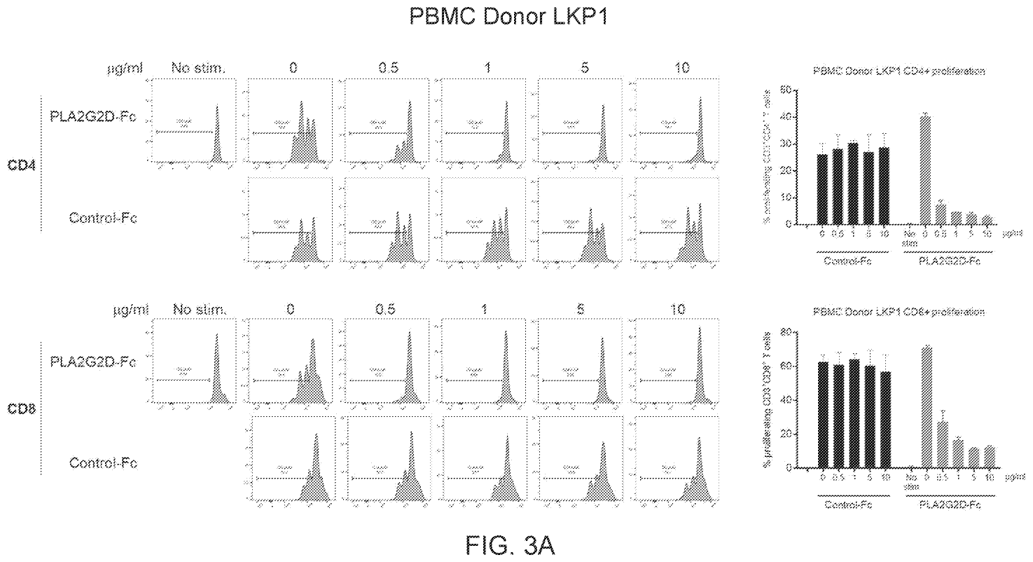

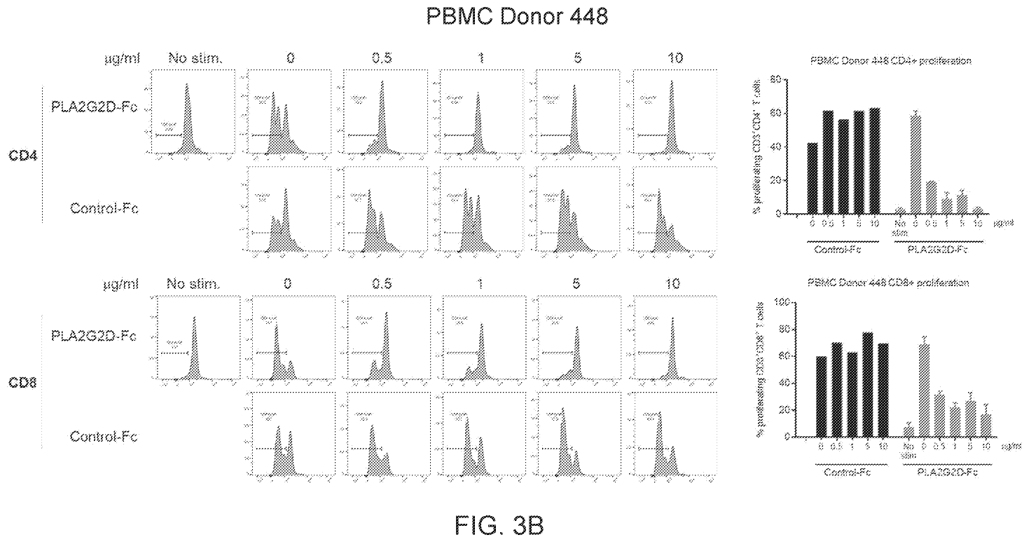

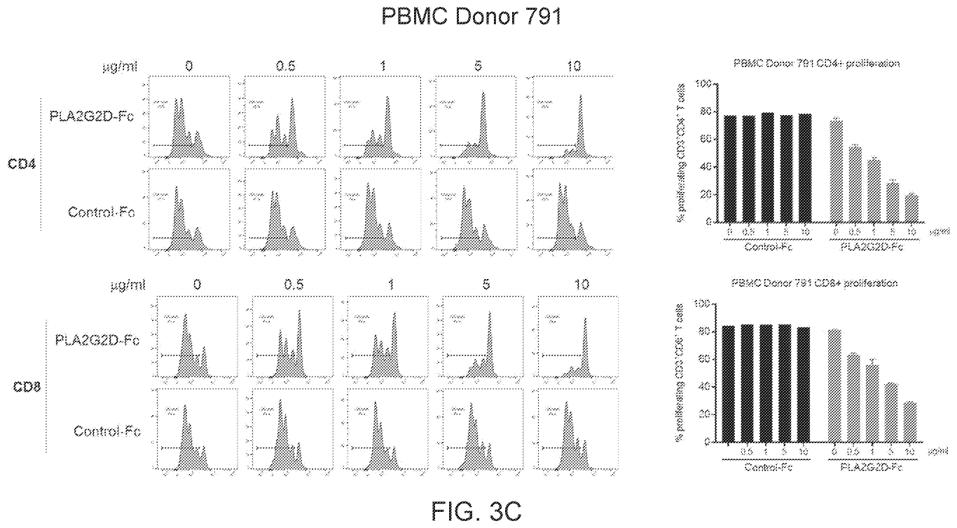

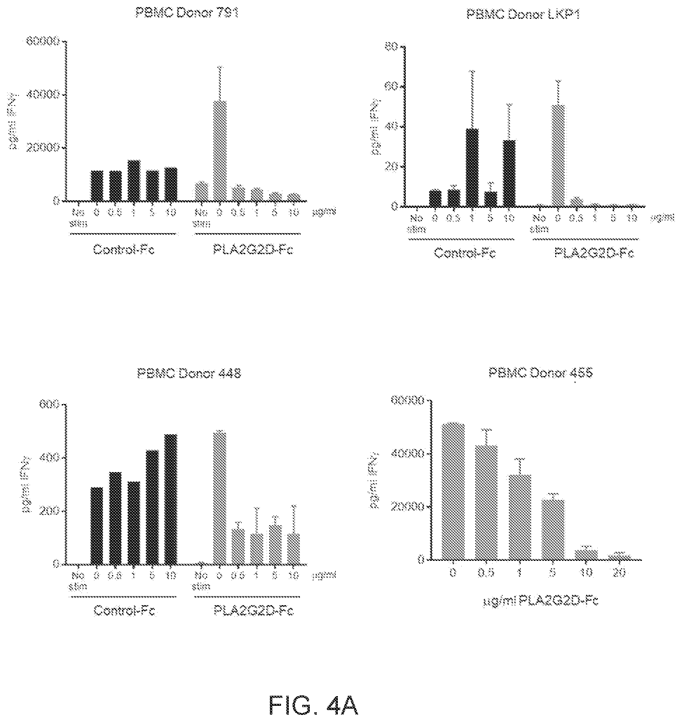

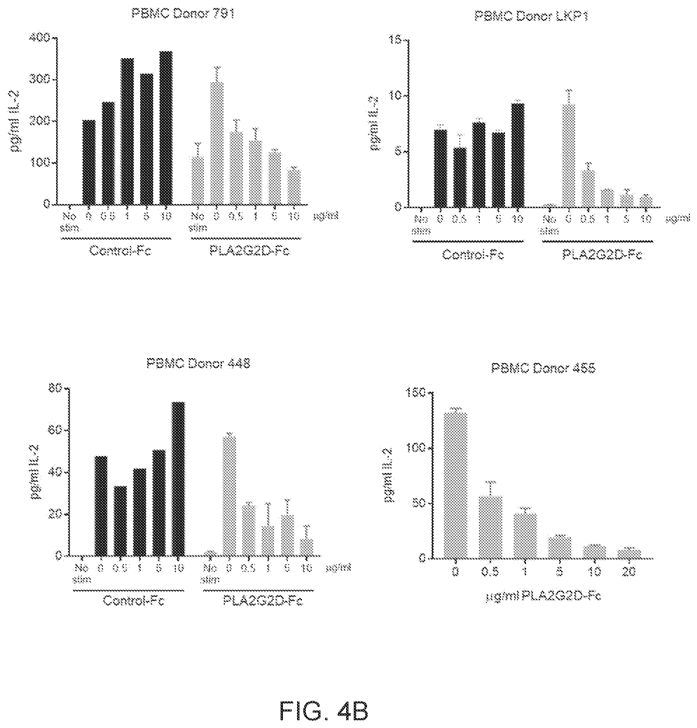

The addition of soluble PLA2G2D-Fc dose dependently suppressed CD4+ and CD8+ T cell proliferation in stimulated PBMC cultures, as measured by CFSE generational tracing. FIGS. 2A-2B show CFSE histograms and quantitation of T cell proliferation for one PBMC donor with increasing concentrations of PLA2G2D-Fc. FIGS. 3A-3C show CFSE histograms and quantitation of proliferation for three more independent PBMC donors in which increasing concentrations of soluble PLA2G2D-Fc protein dose dependently suppressed CD4+ and CD8+ T cell proliferation, whereas equivalent concentrations of Control-Fc protein had no significant effect. FIGS. 4A-4B show that, consistent with T cell proliferation, the levels of IFNγ and IL-2 in the different PBMC cultures were similarly dose-dependently and significantly decreased by increasing concentrations of soluble PLA2G2D-Fc protein, but not control-Fc.

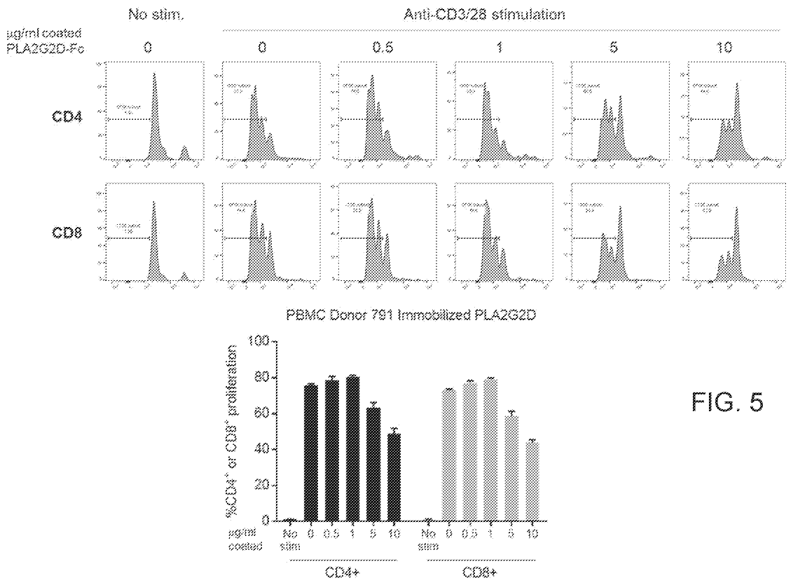

To assess the effect of immobilized PLA2G2D protein on T cell proliferation in PBMC cultures, the same assay was utilized except 100 μl of 0-10 μg/ml human PLA2G2D-Fc or control human IgG1-Fc protein was coated overnight in PBS at 4° C. on 96-well flat bottom plates the day before PBMC were prepared and added along with anti-CD3 and anti-CD28 antibodies. FIG. 5 shows that immobilized human PLA2G2D-Fc protein coated on the plate surface also suppressed CD4+ and CD8+ T cell proliferation in stimulated PBMC cultures.

To evaluate the effect of PLA2G2D protein on isolated T cells, 96-well flat bottom plates were coated overnight at 4° C. with 0-10 μg/ml of human PLA2G2D-Fc or control human IgG1-Fc protein as well as with 1 μg/ml anti-CD3 (OKT3) and 0.2 μg/ml anti-CD28 (CD28.2) in 100 μl of PBS. The following day, plates were washed twice with PBS before addition of T cells. T cells were isolated from PBMC using a Pan T Cell Isolation Kit (Miltenyi) and labeled with CFSE as described above. 1×10⁵ purified and labeled T cells were added to each well of the coated plate in a final volume of 200 μl RPMI. T cells were allowed to proliferate for 72 hours at 37° C. before supernatants were collected for IFNγ and IL-2 analysis by MSD and proliferation was analyzed by FACS.

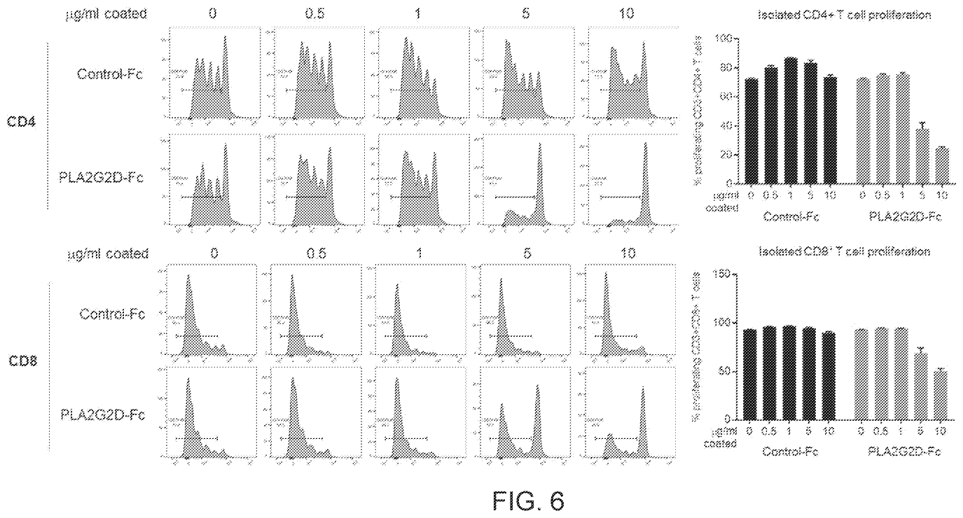

FIG. 6 shows that immobilized human PLA2G2D-Fc protein dose-dependently suppresses proliferation of isolated T cell cultures in the presence of anti-CD3 and anti-CD28 stimulation. However, when soluble PLA2G2D-Fc protein was added to isolated T cell cultures instead of being coated on plates, it had no suppressive effect on T cells (data not shown). Collectively these results suggest that in order for PLA2G2D to elicit functional suppression of T cells, it requires cross-linking by antigen presenting cells or by immobilization on a plate surface.

### Example 3. T Cell Suppression by PLA2G2D

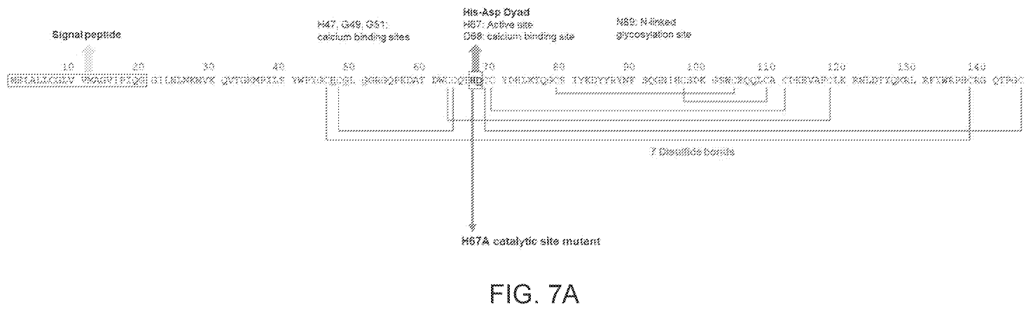

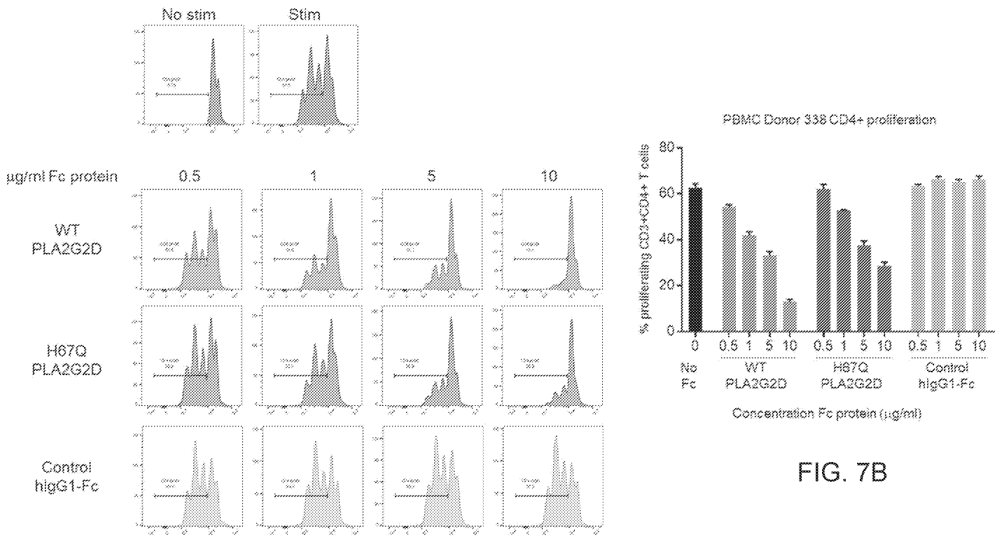

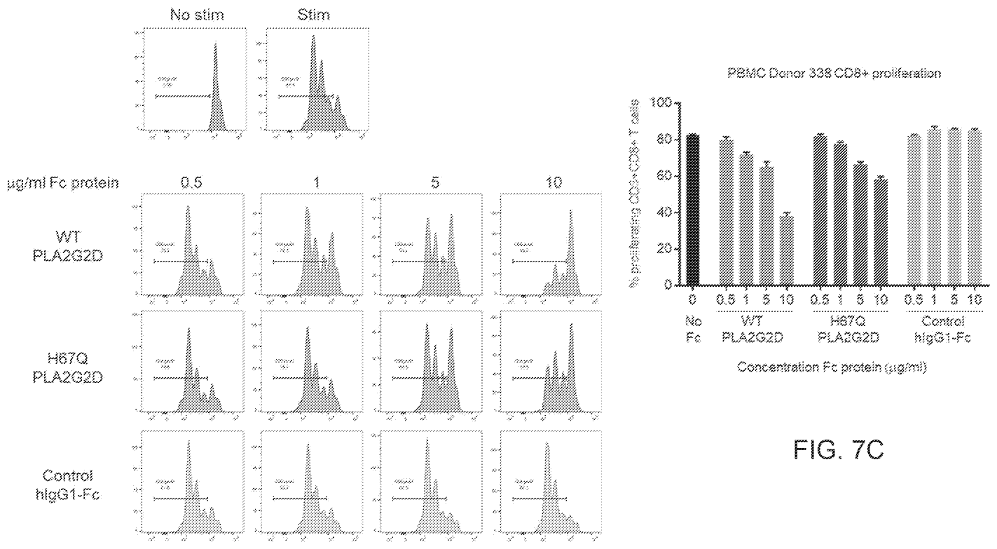

Human PLA2G2D is a 145 amino acid secreted protein consisting of an N-terminal 20 residue signal peptide, a highly conserved Ca²⁺-binding site and a catalytic His-Asp dyad. In addition to these elements, human PLA2G2D features seven disulfide bonds which contribute to a high degree of stability. FIG. 7A shows general structural and functional features of interest of the human PLA2G2D protein.

To evaluate whether PLA2G2D enzyme activity is required for its immune suppressive function, an H67Q mutation was introduced to the highly conserved catalytic His67-Asp68 dyad of human PLA2G2D. Briefly, a human PLA2G2D cDNA fused in frame to a human IgG1-Fc cDNA on the C terminus was synthesized with a CAC→CAG point mutation corresponding to a His-Gin substitution at residue 67. The construct was cloned into a high expression mammalian vector and transfected into HEK293 cells. Secreted human PLA2G2D-H67Q-Fc protein contained in the supernatant was purified by Protein A affinity chromatography. Purified PLA2G2D-H67Q-Fc protein was used in PBMC cultures as described above and compared against wild type PLA2G2D-Fc and control human IgG1-Fc for T cell suppressive activity.

FIGS. 7B-7C show that the PLA2G2D-H67Q-Fc catalytic mutant retains most of the immune suppressive function on CD4⁺ and CD8⁺ T cells at the dose of 0.5 μg/ml to 5 μg/ml, and exhibited a significantly decreased suppression at the dose of 10 μg/ml.

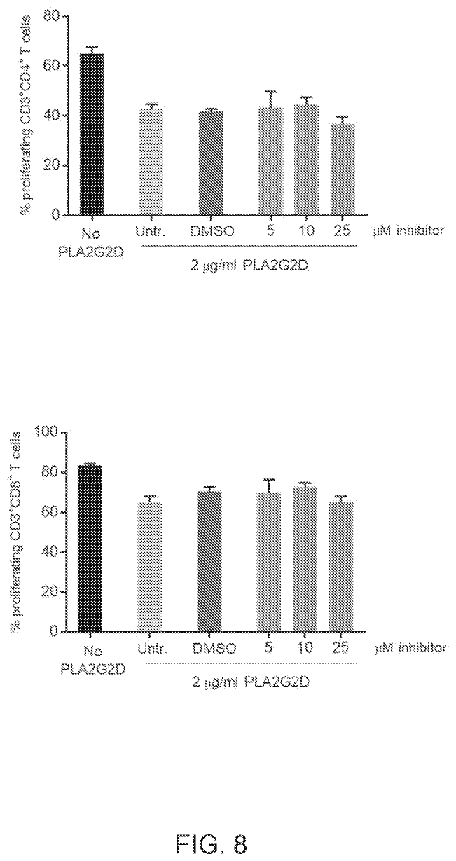

Alternatively, PBMC culture T cell proliferation in the presence of wild type PLA2G2D-Fc protein, as described above, was assessed with the addition of 0-25 μM of the sPLA2 inhibitor LY315920, or DMSO control. FIG. 8 shows that LY315920 does not reverse immune suppression induced by PLA2G2D.

### Example 4. Binding of PLA2G2D to Activated T Cells

To determine whether PLA2G2D elicits immune suppression by directly binding to T cells in vitro, the level of PLA2G2D binding was assessed on resting and activated primary human T cells. T cells were isolated from PBMC using a Pan T Cell Isolation Kit (Miltenyi) and cultured in the presence or absence of beads loaded with anti-human CD2, CD3, and CD28 antibodies (human T Cell Activation/Expansion Kit, Miltenyi) in RPMI for 48 hours at 37° C. Stimulated or unstimulated T cells were then harvested, washed, and incubated with 0-10 μg/ml of human PLA2G2D-Fc protein or human IgG1-Fc protein for 30 minutes at 4° C. Cells were washed 3 times with PBS and then stained with Alexa 488-conjugated goat anti-human IgG1 antibodies (Invitrogen) for 30 minutes at 4° C., washed, then analyzed by FACS.

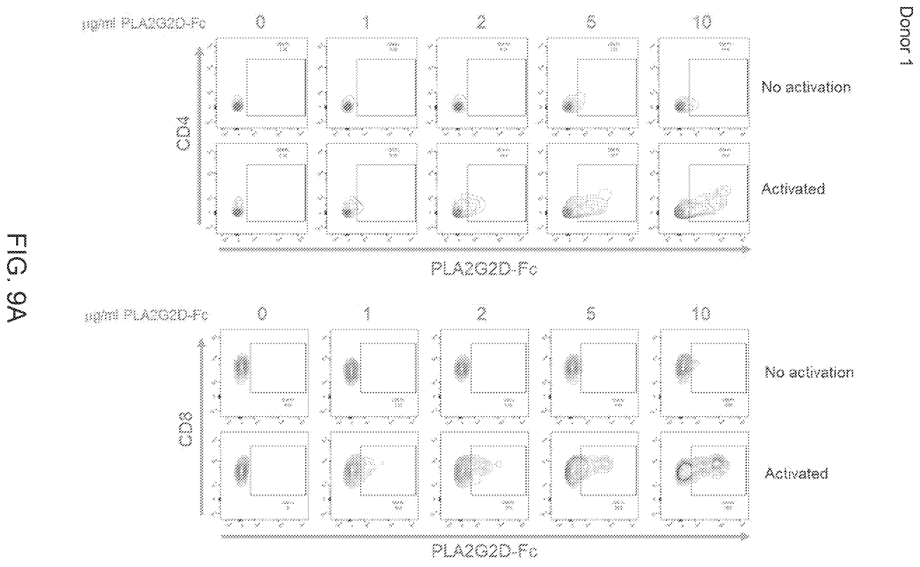

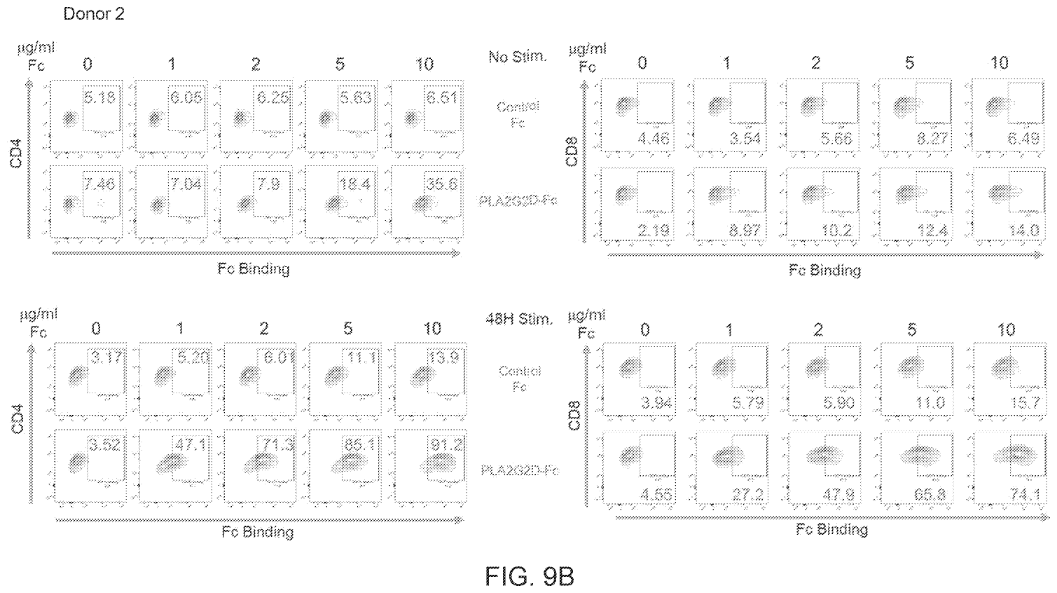

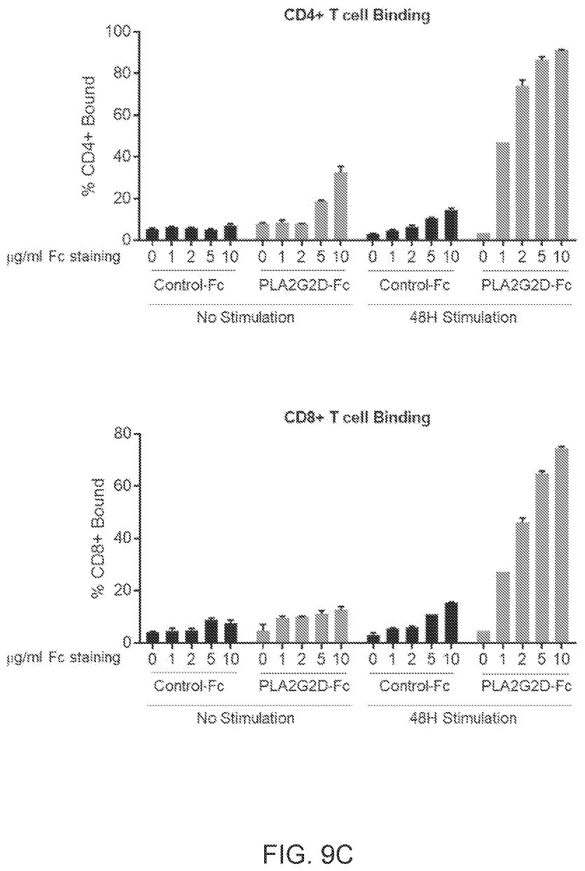

FIGS. 9A-9C show that human PLA2G2D-Fc preferentially binds activated CD4⁺ and CD8⁺ T cells in two different donor T cells compared with control human IgG1-Fc protein. PLA2G2D binds unstimulated T cells to a small degree, but binding is dramatically increased upon T cell stimulation. FIG. 9C shows a quantitative representation of PLA2G2D-Fc binding to stimulated T cells. Addition of heparin sulfate proteoglycan (HSPG) partially reduced PLA2G2D-Fc binding of T cells but did not alter suppression of CD4+ and CD8+ T cell proliferation (data not shown). This suggests that the immune suppression potentially associated with the binding of PLA2G2D to T cells is not dependent on the binding through heparin sulfate on cell surface.

### Example 5. Syngeneic Tumor Growth in PLA2G2D Deficient Mice

PLA2G2D knockout mice were generated by deleting exon 2 of the mouse Pla2g2d gene from C57BL6 mice using CRISPR/Cas9-mediated gene editing. To confirm the absence of a functional P1a2g2d gene in these mice, spleens from wild-type and knockout mice were harvested and total RNA isolated using TRIZol (Invitrogen). Total RNA was subjected to real-time RT-PCR to detect Pla2g2d mRNA. Hprt1 (hypoxanthine-guanine phosphoribosyltransferase) mRNA levels were also measured as a control. The results demonstrated that the knockout mice were deficient in Pla2g2d expression.

To evaluate the effect of Pla2g2d deficiency on tumor growth, the murine syngeneic tumor cell lines MC38 (colon adenocarcinoma), B16F10 (melanoma), and E.G7-OVA (T cell lymphoma) were implanted into age-matched wild-type C57BL6 (WT) or PLA2G2D knockout mice. 1×10⁶ MC38 or E.G7-OVA cells, or 5×10⁵ B16F10 cells suspended in 100 ul PBS were subcutaneously injected into WT (n=16) or PLA2G2D knockout mice (n=16), and tumor growth was monitored every 2 or 3 days. Tumor volume was calculated using the formula, tumor volume=0.5×length×width² Body weights were also monitored weekly. Mice were sacrificed after 3-4 weeks, or upon reaching designated endpoints.

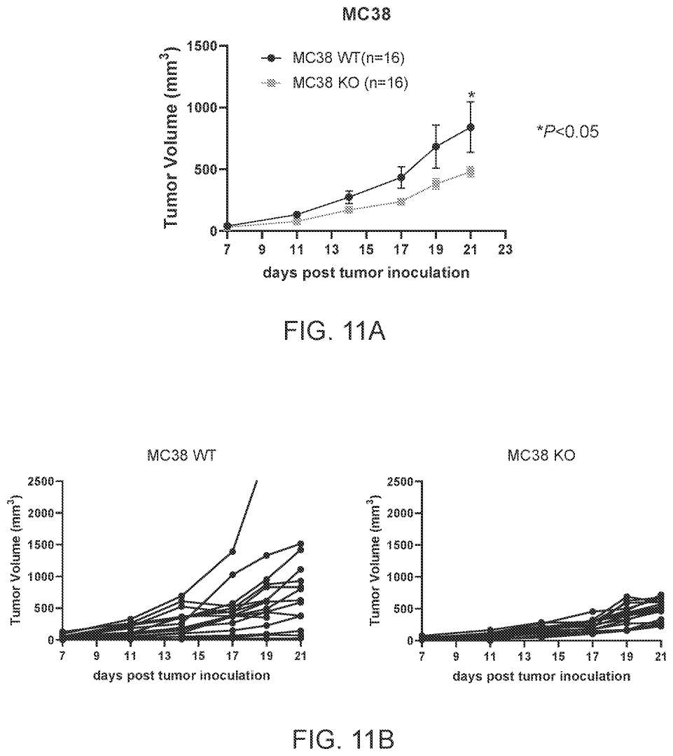

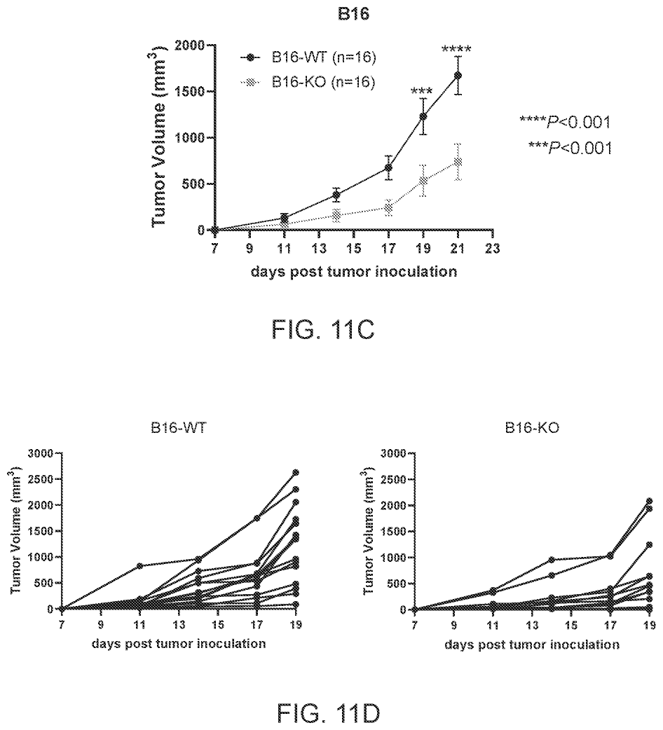

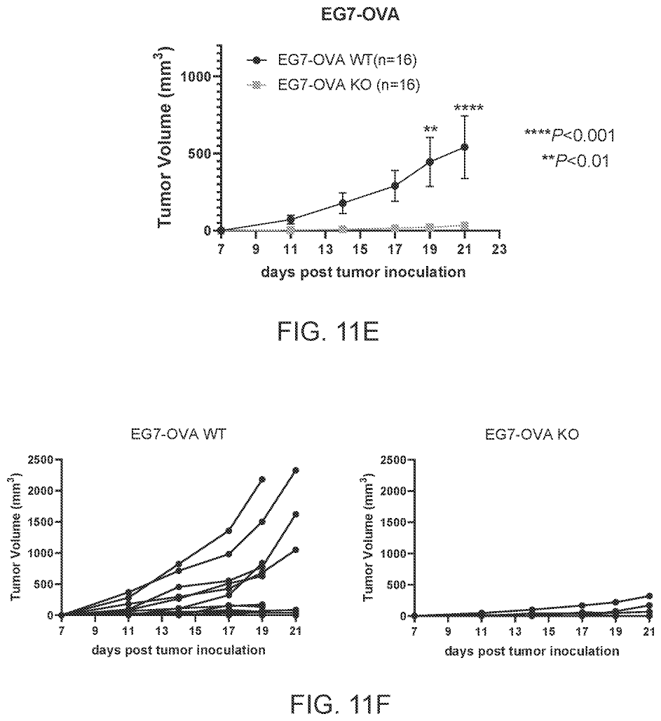

As shown in FIGS. 11A-F, tumor growth of all three syngeneic tumor cell lines was significantly reduced in PLA2G2D knockout mice compared to wild type mice, indicating a role for PLA2G2D in tumor progression and supporting targeted inhibition of PLA2G2D as a potential immunotherapy.

### Example 6. Perturbation of PLA2G2D Immunosuppressive Function by Anti-PLA2G2D Monoclonal Antibodies

To demonstrate whether PLA2G2D immunosuppression can be neutralized and reversed by anti-PLA2G2D antibodies, we developed PLA2G2D-binding monoclonal antibodies by immunizing mice and generating hybridomas.

### Blocking of PLA2G2D Binding to Activated T Cells

As shown in the above example (FIGS. 9A-9C), we found that PLA2G2D preferentially binds to activated T cells, but only minimally to resting T cells, suggesting that PLA2G2D can impart suppressive signaling by directly binding T cells upon their activation. We therefore devised an assay to determine whether anti-PLA2G2D antibodies could hinder PLA2G2D binding to activated T cells and potentially block immune suppression. T cells within human PBMC cultures were stimulated with anti-CD3 and anti-CD28 antibodies (Invitrogen) for 24 hours at 37° C. PBMC cultures were then harvested, washed, and incubated with 2 μg/ml of human PLA2G2D-Fc protein or control human IgG1-Fc protein for 30 minutes at 4° C. in the presence of 10 μg/ml of PLA2G2D antibodies (developed in house) or mouse IgG2a isotype control antibody (Invitrogen). Cells were washed 3 times with PBS and then stained with PE-conjugated goat anti-human IgG1 antibodies (Invitrogen) alongside fluorophore-conjugated anti-CD3, anti-CD4, and anti-CD8 antibodies (Biolegend) and Live/Dead Fixable dead cell stain (Molecular Probes) for 30 minutes at 4° C., washed, then analyzed by FACS.

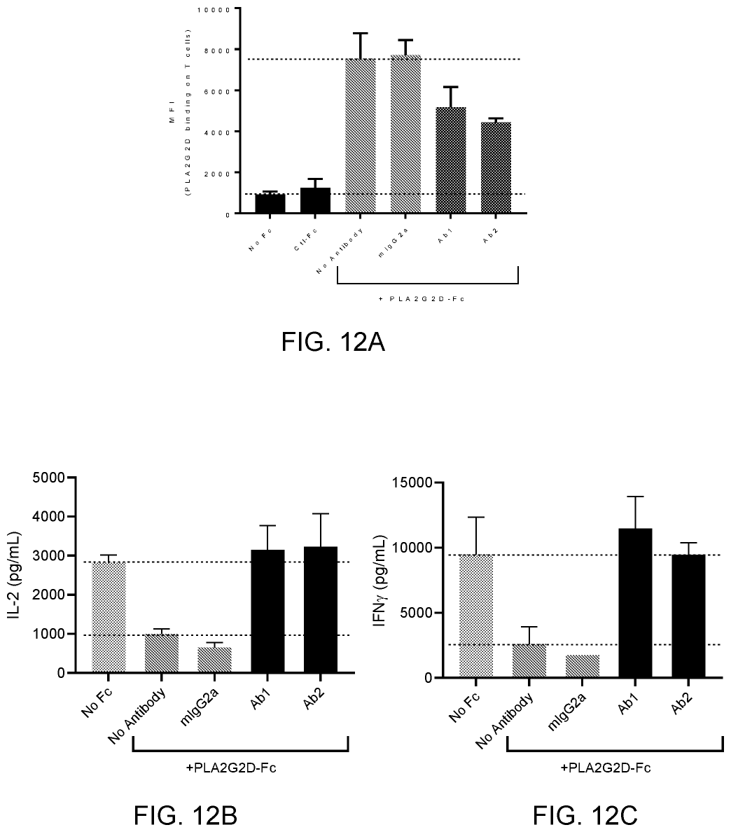

FIG. 12A shows that that two representative PLA2G2D-binding antibodies are able to reduce binding of PLA2G2D to activated T cells, as measured by the mean fluorescence intensity (MFI) of PLA2G2D staining on gated T cells. In contrast, a control mouse IgG2a isotype control had no effect on PLA2G2D binding.

### Reversal of PLA2G2D-Dependent T Cell Suppression in PBMC Culture

To determine whether PLA2G2D antibodies could neutralize PLA2G2D-dependent suppression of T cell function, we used an approach similar to the one we took to demonstrate PLA2GD suppressive activity above (FIGS. 4A-4B). Triplicate wells containing 2×10⁵ PBMC per well of a 96 well round bottom plate were stimulated with 1 μg/ml anti-CD3 (OKT3, Invitrogen) and 0.2 μg/ml anti-CD28 (CD28.2, Invitrogen) in the presence of 1 μg/ml soluble human PLA2G2D-Fc protein (Sino Biological) and 10 μg/ml PLA2GD antibodies or control mIgG2a isotype control antibody (Invitrogen) as indicated in a final volume of 200 μl RPMI (Corning). PBMC cultures were incubated for 48 hours at 37° C. before the supernatants were harvested and measured for IL-2 and IFNγ levels using MSD V-plex assays (Meso Scale Discovery).

FIGS. 12B-12C show that two representative function-blocking PLA2G2D antibodies are able to rescue PLA2G2D-mediated suppression of IL-2 and IFNγ secretion levels in this assay.

### Example 7. Use of PLA2G2D Antibody to Treat Tumor

To evaluate the effect of PLA2G2D antibodies on tumor growth, the murine syngeneic tumor cell lines MC38 (colon adenocarcinoma), B16F10 (melanoma), and E.G7-OVA (T cell lymphoma) are implanted into age-matched wild-type C57BL6 mice. 1×10⁶ MC38 or E.G7-OVA cells, or 5×10⁵ B16F10 cells suspended in 100 ul PBS are subcutaneously injected into C57BL6 mice. When tumor volumes are in the range of 50-150 mm³, mice are randomized into control and treatment groups. PLA2G2D antibodies are administered on days 1, 4, 7, and 11 post-randomization at a dose of 10 mg/kg by IP injection. Tumor growth is monitored every 2 or 3 days. Tumor volume is calculated using the formula-tumor volume=0.5×length×width². Body weights are also monitored weekly. Mice are sacrificed after 3-4 weeks, or upon reaching designated endpoints.

SEQUENCE TABLE

| SEQ | | |
|---|---|---|
| ID NO | Description | Sequences |
| 1. | Human PLA2G2D | MELALLCGLVVMAGVIPIQGGILNLNKMVKQVTGKMPILSY |
| | (with signal | WPYGCHCGLGGRGQPKDATDWCCQTHDCCYDHLKTQGCSI |
| | peptide) | YKDYYRYNFSQGNIHCSDKGSWCEQQLCACDKEVAFCLKR |
| | UniProtKB/Swiss- | NLDTYQKRLRFYWRPHCRGQTPGC |
| | Prot: Q9UNK4.2 | |
| 2. | Human PLA2G2D | GILNLNKMVKQVTGKMPILSYWPYGCHCGLGGRGQPKDAT |
| | (without signal | DWCCQTHDCCYDHLKTQGCSIYKDYYRYNFSQGNIHCSDKG |
| | peptide) | SWCEQQLCACDKEVAFCLKRNLDTYQKRLRFYWRPHCRGQ |
| | GenBank: | TPGC |
| | EAW94915.1 | |
| 3. | PLA2G2D H67A | MELALLCGLVVMAGVIPIQGGILNLNKMVKQVTGKMPILSY |
| | (with signal | WPYGCHCGLGGRGQPKDATDWCCQTADCCYDHLKTQGCSI |
| | peptide) | YKDYYRYNFSQGNIHCSDKGSWCEQQLCACDKEVAFCLKR |
| | | NLDTYQKRLRFYWRPHCRGQTGC |
| 4. | PLA2G2D H47A | GILNLNKMVKQVTGKMPILSYWPYGCHCGLGGRGQPKDAT |
| | (without signal | DWCCQTADCCYDHLKTQGCSIYKDYYRYNFSQGNIHCSDKG |
| | peptide) | SWCEQQLCACDKEVAFCLKRNLDTYQKRLRFYWRPHCRGQ |
| | | TGC |
| 5. | Human PLA2G2D | MELALLCGLVVMAGVIPIQGGILNLNKMVKQVTGKMPILSY |
| | GenBenk: | WPYGCHCGLGGRGQPKDATDWCCQTHDCCYDHLKTQGCGI |
| | AAQ88969.1 | YKDNNKSSIHCMDLSQRYCLMAVFNVIYLENEDSE |
| 6. | PLA2G2D | GILNLNKMVKQVTGKMPILSYWPYGCHCGLGGRGQPKDAT |
| | (without signal | DWCCQTHDCCYDHLKTQGCGIYKDNNKSSIHCMDLSQRYC |
| | peptide) | LMAVFNVIYLENEDSE |
| 7. | PLA2G2D H67A | MELALLCGLVVMAGVIPIQGGILNLNKMVKQVTGKMPILSY |
| | (with signal | WPYGCHCGLGGRGQPKDATDWCCQTADCCYDHLKTQGCGI |
| | peptide) | YKDNNKSSIHCMDLSQRYCLMAVFNVIYLENEDSE |
| 8. | PLA2G2D H47A | GILNLNKMVKQVTGKMPILSYWPYGCHCGLGGRGQPKDAT |
| | (without signal | DWCCQTADCCYDHLKTQGCGIYKDNNKSSIHCMDLSQRYC |
| | peptide) | LMAVFNVIYLENEDSE |
| 9. | PLA2G2D G80S | MELALLCGLVVMAGVIPIQGGILNLNKMVKQVTGKMPILSY |
| | (with signal | WPYGCHCGLGGRGQPKDATDWCCQTHDCCYDHLKTQGCSI |
| | peptide) | YKDNNKSSIHCMDLSQRYCLMAVFNVIYLENEDSE |
| 10. | PLA2G2D G60S | GILNLNKMVKQVTGKMPILSYWPYGCHCGLGGRGQPKDAT |
| | (without signal | DWCCQTHDCCYDHLKTQGCGIYKDNNKSSIHCMDLSQRYC |
| | peptide) | LMAVFNVIYLENEDSE |
| 11. | PLA2G2D H67A | MELALLCGLVVMAGVIPIQGGILNLNKMVKQVTGKMPILSY |
| | & G80S | WPYGCHCGLGGRGQPKDATDWCCQTADCCYDHLKTQGCSI |
| | (with signal | YKDNNKSSIHCMDLSQRYCLMAVFNVIYLENEDSE |
| | peptide) | |
| 12. | PLA2G2D H47A | GILNLNKMVKQVTGKMPILSYWPYGCHCGLGGRGQPKDAT |
| | & G60S | DWCCQTADCCYDHLKTQGCSIYKDNNKSSIHCMDLSQRYCL |
| | (with signal | MAVFNVIYLENEDSE |
| | peptide) | |
| 13. | Linker | (G)ₙ, n >= 1 |
| 14. | Linker | (GS)ₙ, 20 >= n >= 1 |
| 15. | Linker | (GSGGS)ₙ, 8 >= n >= 1 |
| 16. | Linker | (GGGGS)ₙ, 8 >= n >= 1 |
| 17. | Linker | (GGGS)ₙ, 8 >= n >= 1 |
| 18. | Linker | (GGGGS)₃ |
| 19. | Linker | (GGGGS)₆ |
| 20. | Linker | (GSTSGSGKPGSGEGS)ₙ |
| | | 3 >= n >= 1 |
| 21. | Linker | A(EAAAK)₄A |
| 22. | PLA2G2D (same | MELALLCGLVVMAGVIPIQGGILNLNKMVKQVTGKMPILSY |
| | as SEQ ID NO: 1) | WPYGCHCGLGGRGQPKDATDWCCQTHDCCYDHLKTQGCSI |
| | | YKDYYRYNFSQGNIHCSDKGSWCEQQLCACDKEVAFCLKR |
| | | NLDTYQKRLRFYWRPHCRGQTPGC |
| 23. | PLA2G2D (as | ILNLNKMVKQVTGKMPILSYWPYGCHCGLGGRGQPKDATD |
| | compared to | WCCQTHDCCYDHLKTQGCSIYKDYYRYNFSQGNIHCSDKGS |
| | PLA2G1B as in | WCEQQLCACDKEVAFCLKR |
| | FIG. 10B) | |
| 24. | PLA2G1B | VWQFRKMIKCVIPGSDPFLEYNNYGCYCGLGGSGTPVDELD |
| | | KCCQTHDNCYDQAKKLDSCKFLLDNPYTHTYSYSCSGSAITC |
| | | SSKNKECEAFICNCDRNAAICFSK |
| 25. | PLA2G2D (as | MELALLCGLVVMAGVIPIQGGILNLNKMVKQVTGKMPILSY |
| | compared to | WPYGCHCGLGGRGQPKDATDWCCQTHDCCYDHLKTQGCSI |
| | PLA2G2A as in | YKDYYRYNFSQGNIHCSDKGSWCEQQLCACDKEVAFCLKR |
| | FIG. 10B) | NLDTYQKRLRFYWRPHCRGQTPGC |
| 26. | PLA2G2.A | MKTLLLLAVIMIFGLLQAHGNLVNFHRMIKLTTGKEAALSYG |
| | | FYGCHCGVGGRGSPKDATDRCCVTHDCCYKRLEKRGCGTK |
| | | FLSYKFSNSGSRITCAKQDSCRSQLCECDKAAATCFARNKTT |
| | | YNKKYQYYSNKHCRGSTPRC |
| 27. | PLA2G2D (as | PIQGGILNLNKMVKQVTGKMPILSYWPYGCHCGLGGRGQPK |
| | compared to | DATDWCCQTHDCCYDHLKTQGCSIYKDYYRYNFSQGNIHCS |
| | PLA2G2C as in | DKGSWCEQQLCACDKEVAFCLKRNLDTYQKRLRFYWRPHC |
| | FIG. 10B) | RGQTPGC |
| 28. | PLA2G2C | PTHSSFWQFQRRVKHITGRSAFFSYYGYGCYCGLGDKGIPVD |
| | | DTDRHSPSSPSPYEKLKEFSCQPVLNSYQFHIVNGAVVCGCTL |
| | | GPGASCHCRLKACECDKQSVHCFKESLPTYEKNFKQFSSQPR |
| | | CGRHKPWC |
| 29. | PLA2G2D (as | IQGGILNLNKMVKQVTGKMPILSYWPYGCHCGLGGRGQPKD |
| | compared to | ATDWCCQTHDCCYDHLKTQGCSIYKDYYRYNFSQGNIHCSD |
| | PLA2G2E as in | KGSWCEQQLCACDKEVAFCLKRNLDTYQKRLRFYWRPHCR |
| | FIG. 10B) | GQTPGC |
| 30. | PLA2G2E | VTGNLVQFGVMIEKMTGKSALQYNDYGCYCGIGGSHWPVD |
| | | QTDWCCHAHDCCYGRLEKLGCEPKLEKYLFSVSERGIFCAG |
| | | RTTCQRLTCECDKRAALCFRRNLGTYNRKYAHYPNKLCTGP |
| | | TPPC |
| 31. | PLA2G2D (as | GGILNLNKMVKQVTGKMPILSYWPYGCHCGLGGRGQPKDA |
| | compared to | TDWCCQTHDCCYDHLKTQGCSIYKDYYRYNFSQGNIHCSDK |
| | PLA2G2F as in | GSWCEQQLCACDKEVAFCLKRNLDTYQKRLRFYWRPHCRG |
| | FIG. 10B) | QTPGC |
| 32. | PLA2G2F | GSLLNLKAMVEAVTGRSAILSFVGYGCYCGLGGRGQPKDEV |
| | | DWCCHAHDCCYQELFDQGCHPYVDHYDHTIENNTEIVCSDL |
| | | NKTECDKQTCMCDKNMVLCLMNQTYREEYRGFLNVYCQGP |
| | | TPNC |
| 33. | PLA2G2D | MELALLCGLVVMAGVIPIQGGILNLNKMVKQVTGKMPILSY |
| | (Human) (Same as | WPYGCHCGLGGRGQPKDATDWCCQTHDCCYDHLKTQGCSI |
| | SEQ ID NO: 1) | YKDYYRYNFSQGNIHCSDKGSWCEQQLCACDKEVAFCLKR |
| | | NLDTYQKRLRFYWRPHCRGQTPGC |
| 34. | PLA2G2D | MRLALLCGLLLAGITATQGGLLNLNKMVTHMTGKKAFFSY |
| | (mouse) | WPYGCHCGLGGKGQPKDATDWCCQKHDCCYAHLKIDGCK |
| | | SLTDNYKYSISQGTIQCSDNGSWCERQLCACDKEVALCLKQ |
| | | NLDSYNKRLRYYWRPRCKGKTPAC |
| 35. | PLA2G2D (rat) | MRLALLCGLLLAGITATQGGLLNLNKMVNHMTGKKAFFSY |
| | | WPYGCHCGFGGKGQPKDATDWCCQKHDCCYAHLKIDGCKS |
| | | LTDNYKYSISEGVIQCSDQGSWCERQLCACDKEVALCLKQN |
| | | LESYNKRLRYYWRPRCKGQTPTC |
| 36. | PLA2G2D | MQLALLCGLVVMAGVIPIQGGILNLNKMVKQVTGKMPLFFY |
| | (rhesus) | WPYGCYCGPGGRGQPKDATDWCCQTHDCCYDHLKTHGCCI |
| | | HTDHYRYSFSHGDIHCSDKGSWCEQQLCACDKEVAFCLKRN |
| | | LDTYKKRLRFYWRPRCQGQTPGC |
| 37. | PLA2G2D | MELALLCGLVVMAGVIPIQGGILNLNKMVKQVTGKMPILFY |
| | (chimp) | WPYGCHCGLGGRGQPKDATDWCCQTHDCCYDHLKTQGCGI |
| | | YKDYYRYNFSQGNIHCSDKGSWCEQQLCACDKEVAFCLKR |
| | | NLDTYQKRLRFYWRPHCRGQTPGC |

1. A method of treating a cancer or viral infection in an individual, comprising administering into the individual an effective amount of an antagonist targeting PLA2G2D signaling pathway.

2. The method of claim 1, wherein the antagonist is an antagonist inhibits or downregulates PLA2G2D.

3. The method of claim 2, wherein the PLA2G2D is a human PLA2G2D.

4. The method of claim 2 or claim 3, wherein the antagonist decreases enzymatic activity level of PLA2G2D.

5. The method of claim 4, wherein the antagonist targeting PLA2G2D signaling pathway blocks a catalytic site on PLA2G2D.

6. The method of claim 5, wherein the antagonist targets the H67 catalytic site on a human PLA2G2D according to SEQ ID NO: 1 or 5.

7. The method of any one of claims 1-3, wherein the antagonist comprises a siRNA, an miRNA, an antisense RNA, or a gene editing system.

8. The method of any one of claims 1-3, wherein the antagonist blocks the binding of PLA2G2D to an immune cell.

9. The method of claim 8, wherein the immune cell is a T cell.

10. The method of any one of claims 1-3, wherein the antagonist comprises an anti-PLA2G2D antibody.

11. The method of claim 10, wherein the anti-PLA2G2D antibody is a monoclonal antibody.

12. The method of claim 10, wherein the antagonist is a fusion protein or immunoconjugate further comprising a second moiety.

13. The method of claim 12, wherein the second moiety comprises a cytokine.

14. The method of any one of claims 1-3, wherein the antagonist comprises an inhibitory PLA2G2D polypeptide that blocks the binding of PLA2G2D to an immune cell.

15. The method of claim 14, wherein the inhibitory PLA2G2D polypeptide binds to the immune cell with a greater affinity than a wildtype PLA2G2D.

16. The method of claim 15, wherein the immune cells is a T cell.

17. The method of any one of claims 14-16, wherein inhibitory PLA2G2D polypeptide further comprises a stabilizing domain.

18. The method of claim 17, wherein the stabilizing domain is an Fc domain.

19. The method of any one of claims 14-18, wherein the inhibitory PLA2G2D polypeptide has a length of about 50 to about 200 amino acids.

20. The method of any one of claims 14-19, wherein the inhibitory PLA2G2D polypeptide has a) a mutation at a position corresponding to histidine at position 67 (H67) according to SEQ ID NO: 1 or 5 or b) a mutation at the position corresponding to glycine at position 80 (G80) according to SEQ ID NO: 5.

21. The method of claim 20, wherein the inhibitory PLA2G2D polypeptide comprises an amino acid sequence selected from the group consisting of SEQ ID NOs: 3, 4, and 7-12 or a variant thereof.

22. The method of any one of claims 1-21, wherein the disease or condition is a cancer.

23. The method of claim 22, wherein the cancer is a solid tumor.

24. The method of claim 22 or claim 23, wherein the cancer is an advanced or malignant tumor.

25. The method of any one of claims 22-24, wherein the cancer has an increased expression level of PLA2G2D.

26. The method of any one of claims 22-25, wherein the cancer is selected from the group consisting of lung cancer, breast cancer, liver cancer, gastric cancer, cervical cancer, endometrial cancer, thyroid cancer, colorectal cancer, head and neck cancer, pancreatic cancer, renal cancer, prostate cancer, urothelial cancer, testis cancer, ovarian cancer and melanoma.

27. The method of any one of claims 1-21, wherein the disease or condition is a viral infection.

28. The method of claim 27, wherein the expression level of PLA2G2D at an infected site is higher than that of an uninfected site.

29. The method of any one of claims 1-28, wherein the method further comprises administering a second agent.

30. The method of claim 29, wherein the second agent is selected from the group consisting of a chemotherapeutic agent, an immunomodulator, an anti-angiogenesis agent, a growth inhibitory agent, and an antineoplastic agent.

31. The method of claim 30, wherein the second agent is an immunomodulator.

32. The method of claim 31, wherein the immunomodulator is an immune checkpoint inhibitor.

33. The method of claim 28, wherein the immune checkpoint inhibitor specifically target PD-L1, PD-L2, CTLA4, PD-L2, PD-1, CD47, TIGIT, GITR, TIM3, LAG3, CD27, 4-1BB, or B7H4.

34. The method of claim 33, wherein the second agent comprises a cell comprising a chimeric antigen receptor that specifically binds to a tumor antigen.

35. The method of any one of claims 29-34, wherein the antagonist and the second agent are administered simultaneously or concurrently.

36. The method of any one of claims 29-34, wherein the antagonist and the second agent are administered sequentially.

37. The method of any one of claims 1-36, wherein the antagonist and/or the second agent is administered parentally.

38. The method of any one of claims 22-37, wherein the antagonist is administered to the cancer tissue or infection site directly.

39. The method of any one of claims 1-38, wherein the antagonist is administered at a dose of about 0.001 μg/kg to about 100 mg/kg.

40. The method of any one of claims 22-39, wherein the individual has an increased number of immune cells in the cancer tissue or at the infection site after administration of the antagonist.

41. The method of claim 40, wherein the immune cells are T cells.

42. The method of claim 40 or claim 41, wherein the T cells are activated T cells.

43. The method of any one of claim 40-42, wherein the number of immune cells in the cancer tissue or at the infection site is increased by at least about 5% after administration of the antagonist.

44. The method of any one of claims 22-43, wherein immune cells in the cancer tissue or at the infection site produce an increased level of a cytokine after administration of the antagonist.

45. The method of claim 44, wherein the cytokine is IFNγ and/or IL-2.

46. The method of claim 39 or claim 40, wherein the level of the cytokine is increased by at least about 5% after administration of the antagonist.
